(19) United States
(12) Patent Application Publication
(10) Pub. No.: US 2013/0095136 A1
(43) Pub. Date: Apr. 18, 2013

(54) Tetravalent Dengue Vaccines

(71) Applicant: Farshad Guirakhoo, Melrose, MA (US)

(72) Inventor: Farshad Guirakhoo, Melrose, MA (US)

(73) Assignee: Sanofi Pasteur Biologics, LLC, Cambridge, MA (US)

(21) Appl. No.: 13/475,202

(22) Filed: May 18, 2012

Related U.S. Application Data
(63) Continuation of application No. 13/248,289, filed on Sep. 29, 2011, now abandoned.
Application No. 13/248,289 is a continuation of application No. 12/471,962, filed on May 26, 2009, now abandoned.
Application No. 12/471,962 is a continuation of application No. 10/452,610, filed on Jun. 2, 2003, now abandoned.
(60) Provisional application No. 60/385,013, filed on May 31, 2002.

(51) Int. Cl. A61K 39/12

(52) U.S. Cl. CPC A61K 39/12
USPC 424/218.1

(57) ABSTRACT

The invention provides tetravalent Dengue vaccines, and methods of using these vaccines to prevent or to treat Dengue infection.

### CROSS-REFERENCE TO RELATED APPLICATIONS

This application is a continuation of and claims priority from U.S. Ser. No. 13/248,289, filed Sep. 29, 2011, which is continuation of Ser. No. 12/471,962, filed May 26, 2009 (abandoned), which is a continuation of U.S. Ser. No. 10/452,610, filed Jun. 2, 2003 (abandoned), which claims benefit of U.S. Provisional Application No. 60/385,013, filed May 31, 2002, the contents of which are incorporated herein by reference.

### BACKGROUND OF THE INVENTION

This invention relates to tetravalent vaccines against Dengue virus, and methods of using these vaccines to prevent or to treat Dengue virus infection.

Dengue (DEN), a positive stranded RNA virus, is a member of the Flaviviridae family, which contains more than 70 viruses. Dengue viruses are transmitted to humans by mosquitoes (mainly by Aedes aegypti) and are the cause of a growing public health problem worldwide. Fifty to 100 million persons are infected by Dengue virus annually, and rates of infection as high as 6% have been observed in some areas (Gubler, “Dengue and Dengue Hemorrhagic Fever,” CABI Publ., New York, Chapter 1, pp. 1-22, 1997; Burke et al., Am. J. Trop. Med. Hyg. 38:172-180, 1988).

Four serotypes of Dengue virus (DEN1-4) circulate in the Caribbean, Asia, and the Americas. The severe, potentially lethal form of DEN infection [dengue hemorrhagic fever/dengue shock syndrome (DHF/DSS)] is an immunopathological disease occurring in individuals who have sustained sequential infections with different DEN serotypes. Over 3.6 million cases of DHF and 58,000 deaths caused by DHF were reported between 1980 and 1995 (Halstead, “Dengue and Dengue Hemorrhagic Fever,” CABI Publ., New York, Chapter 2, pp. 23-44, 1997). Because of the pathogenesis of DHF/DSS, it is generally thought that a successful DEN vaccine will need to immunize against all four serotypes of Dengue virus simultaneously and induce long-lasting immunity. Despite the extensive efforts that have made towards developing an effective Dengue vaccine since World War II, there is currently no approved DEN vaccine available.

### SUMMARY OF THE INVENTION

The invention provides methods of inducing an immune response to the four serotypes of dengue virus in patients, involving administering to the patients: (i) a chimeric flavivirus including the capsid and non-structural proteins of Yellow Fever virus and the pre-membrane and envelope proteins of Dengue-1 virus (ChimeriVax™-DEN1); (ii) a chimeric flavivirus including the capsid and non-structural proteins of Yellow Fever virus and the pre-membrane and envelope proteins of Dengue-2 virus (ChimeriVax™-DEN2); (iii) a chimeric flavivirus including the capsid and non-structural proteins of Yellow Fever virus and the pre-membrane and envelope proteins of Dengue-3 virus (ChimeriVax™-DEN3); and (iv) a chimeric flavivirus including the capsid and non-structural proteins of Yellow Fever virus and the pre-membrane and envelope proteins of Dengue-4 virus (ChimeriVax™-DEN4).

The methods of the invention involve the administration of equal amounts of each serotype (e.g., 5,5,5,5 or 4,4,4,4 log₁₀ PFU of ChimeriVax™-DEN1, -DEN2, -DEN3, and -DEN4, respectively) or a lower amount of one, two, or other three serotypes relative to the fourth serotype (e.g., at least 5, 10, or 100 fold less). For example, ratios such as 5,5,4,4; 5,5,3,3; 4,4,3,3; 5,5,5,4; 5,5,5,3; 4,4,4,3; 5,5,4,5; 5,5,3,5; or 4,4,3,4 can be used. Criteria that can be used in selecting one or more of these approaches can include determination of whether the chimera includes a mutation or not (see below). For example, if the Dengue-1 and Dengue-2 chimeras include mutations that affect, for example, the level and duration of viremia and/or the immune response, and the other two chimeras do not include such mutations, it may be desirable to include less of the latter two chimeras (e.g., 5,5,4,4 or 5,5,3,3) (see below).

The invention also includes vaccine compositions that contain: (i) a chimeric flavivirus including the capsid and non-structural proteins of Yellow Fever virus and the pre-membrane and envelope proteins of Dengue-1 virus; (ii) a chimeric flavivirus including the capsid and non-structural proteins of Yellow Fever virus and the pre-membrane and envelope proteins of Dengue-2 virus; (iii) a chimeric flavivirus including the capsid and non-structural proteins of Yellow Fever virus and the pre-membrane and envelope proteins of Dengue-3 virus; and (iv) a chimeric flavivirus including the capsid and non-structural proteins of Yellow Fever virus and the pre-membrane and envelope proteins of Dengue-4 virus. The vaccine compositions of the invention include less (e.g., at least 5, 10, or 100 fold less) of one, two, or three chimeras, relative to the other chimeras (e.g., 5,5,3,3; 5,5,3,5; 5,5,5,3; 3,5,5,3; and 5,3,3,3 log₁₀ PFU) (also see above), which can each, optionally, be present in equivalent amounts (e.g., 5,5,5,5; 4,4,4,4).

The invention also includes use of the tetravalent chimera formulations described herein in the prevention and treatment of disease, such as that caused by dengue infection, as well as the use of these formulations in the preparation of medicaments for such use.

The invention provides several advantages. For example, as is discussed above, an optimal approach to vaccinating against Dengue virus requires immunization against all four Dengue serotypes, because individuals who are incompletely immunized or in whom antibody titers to an individual serotype have diminished substantially may be sensitized to a severe immunopathological disease, such as DHS/DSS. Development of a vaccine that can be used to immunize against all four serotypes has been a challenge in this field for many years. This is due, in part, to the phenomenon of viral interference, in which at least one virus in a multivalent vaccine predominates over the others, leading to an imbalanced immune response characterized by under-representation of one or more viruses. As is described further below, this problem has been overcome in the present invention, which can be used to achieve a balanced immune response.

Additional advantages are provided by the fact that the invention can employ YF17D as a live vector, as YF17D (i) has had its safety established for >60 years, during which over 350 million doses have been administered to humans, (ii) induces a long duration of immunity after a single dose, and (iii) induces immunity rapidly, within a few days of inoculation. In addition, the chimeric vaccine viruses of the invention cause an active infection in the treated patients. As the cytokine milieu and innate immune response of immunized individuals are similar to those in natural infection, the antigenic mass expands in the host, properly folded conformational epitopes are processed efficiently, the adaptive immune response is robust, and memory is established. Moreover, the prM and E proteins derived from the target Dengue virus contain the critical antigens for protective humoral and cellular immunity.

Other features and advantages of the invention will be apparent from the following Detailed Description, the Drawings, and the Claims.

### BRIEF DESCRIPTION OF THE DRAWINGS

FIG. 1 is a schematic representation of the three-fragment ligation carried out to generate ChimeriVax-DEN3₀₀ virus.

FIG. 2 shows the production history of working Cell Bank of Aventis Pasteur Vero cells.

FIG. 3 demonstrates the production strategy of ChimeriVax™-DEN pre-Master Seed viruses in Vero LS-10 cells. PP: plaque-purification. After 1^{st} plaque-purification (P3), ten clones (A through J) were picked and stored. After two additional plaque-purifications (P4 and P5), followed by two more passages, final P7 cloned PMS candidates were selected based on their full-genome sequences.

FIG. 4 is a schematic representation of cDNA template and in vitro RNA transcripts to produce ChimeriVax™-DEN1 PMS virus.

FIG. 5 shows chimeric plasmids containing cloned YF 17D and DEN2 PUO 218 sequences, digestion/ligation of fragments, and production of chimeric RNA using SP6 transcription.

FIG. 6 shows the preparation of cDNA template and in vitro RNA transcripts to produce ChimeriVax™-DEN4 chimera.

FIGS. 7A-7C are graphs that show the growth kinetics of ChimeriVax-DEN viruses in Vero cells. FIG. 7A: Growth kinetics of ChimeriVax-DEN1₉₉ P3 and ChimeriVax-DEN1₀₀ large plaque phenotypes at P4. FIG. 7B: Growth kinetics of ChimeriVax-DEN3₀₀ (containing NarI or PVUII restriction sites at E/NS1 junction), ChimeriVax-DEN3₉₉ (containing NarI site at E/NS1 junction), and ChimeriVax-DEN2 control at MOI 0.01. FIG. 7C: Growth kinetics of ChimeriVax-DEN4₀₀ compared to ChimeriVax-DEN4₉₉ uncloned, large, and small plaque phenotypes.

FIG. 8 shows plaque morphology of ChimeriVax™-Den1-4 vaccine bulks using IFF assay on Vero cells.

FIG. 9 represent the mean daily total serum viremia (n=11 monkeys per group) in monkeys inoculated IC with YF-Vax® or ChimeriVax™-DEN1-4.

### DETAILED DESCRIPTION

The invention provides tetravalent Dengue virus vaccines and methods of using these vaccines in the prevention and treatment of Dengue virus infection. As is discussed above, there are four Dengue serotypes (DEN1-4), and optimal vaccination against Dengue virus requires the induction of immunity against all four of these serotypes. The vaccines and methods of the present invention, which are described in more detail below, can be used to achieve such a balanced immune response.

Fully processed, mature virions of flaviviruses, such as Dengue virus and Yellow Fever virus, contain three structural proteins, capsid (C), membrane (M), and envelope (E), and seven non-structural proteins. Immature flavivirions found in infected cells contain pre-membrane (prM) protein, which is a precursor to the M protein. The flavivirus proteins are produced by translation of a single, long open reading frame to generate a polyprotein, followed by a complex series of post-translational proteolytic cleavages of the polyprotein, to generate mature viral proteins (Amberg et al., J. Virol. 73:8083-8094, 1999; Rice, “Flaviviridae,” In Virology, Fields (ed.), Raven-Lippincott, New York, 1995, Volume I, p. 937). The virus structural proteins are arranged in the polyprotein in the order C-prM-E.

The viruses employed in the vaccines and methods of the present invention are live, attenuated chimeric viruses that consist of a first flavivirus (e.g., a Yellow Fever virus) in which a structural protein (or proteins) has been replaced with a corresponding structural protein (or proteins) of a Dengue virus. Preferably, the chimeras consist of a Yellow Fever virus (e.g., the Yellow Fever human vaccine strain YF17D) in which the pre-membrane (prM) and envelope (E) proteins of the Yellow Fever virus have been replaced with the prM and E proteins of a Dengue virus (serotype 1, 2, 3, or 4). Details of making chimeric viruses that can be used in the invention are provided, for example, in International applications PCT/US98/03894 and PCT/US00/32821; Chambers et al., J. Virol. 73:3095-3101, 1999; Guirakhoo et al., J. Virol. 74:5477-5485, 2000; Guirakhoo et al., J. Virology 75(16):7290-7304, 2001; and Guirakhoo et al., Virology 298:146-159, 2002, each of which is incorporated by reference herein in its entirety.

Optionally, chimeras used in the vaccines and methods of the invention can include mutations that impart favorable characteristics to the chimeras. For example, the chimeras can include mutations that decrease viscerotropism. In one example of such a mutation, the lysine at position 204 of the Dengue envelope protein is substituted or deleted. For example, as is described in further detail below, this residue can be replaced with, for example, arginine, to decrease viscerotropism of Yellow Fever virus/Dengue chimeras. Additional mutations that can be included in the chimeras used in the invention are described, for example, in U.S. Patent Application Ser. No. 60/348,949, filed Jan. 15, 2002, which is incorporated herein by reference.

The chimeras used in the vaccines and methods of the present invention can be made using standard methods in the art. For example, an RNA molecule corresponding to the genome of a chimera can be introduced into primary cells, chick embryos, or diploid cell lines, from which (or the supernatants of which) progeny virus can then be purified. Another method that can be used to produce the chimeras employs heteroploid cells, such as Vero cells (Yasumura et al., Nihon Rinsho 21:1201-1215, 1963). In this method, a nucleic acid molecule (e.g., an RNA molecule) corresponding to the genome of a chimera is introduced into the heteroploid cells, virus is harvested from the medium in which the cells have been cultured, harvested virus is treated with a nuclease (e.g., an endonuclease that degrades both DNA and RNA, such as Benzonase™; U.S. Pat. No. 5,173,418), the nuclease-treated virus is concentrated (e.g., by use of ultrafiltration using a filter having a molecular weight cut-off of, e.g., 50-500 kDa), and the concentrated virus is formulated for the purposes of vaccination. Details of this method are provided in U.S. Patent Application Ser. No. 60/348,565, filed Jan. 15, 2002, which is incorporated herein by reference.

Formulation of the chimeric viruses of the vaccines and methods of the invention can be carried out using methods that are standard in the art. Numerous pharmaceutically acceptable solutions for use in vaccine preparation are well known and can readily be adapted for use in the present invention by those of skill in this art. (See, e.g., Remington's Pharmaceutical Sciences (18^{th} edition), ed. A. Gennaro, 1990, Mack Publishing Co., Easton, Pa.) In two specific examples, the chimeras are formulated in Minimum Essential Medium Earle's Salt (MEME) containing 7.5% lactose and 2.5% human serum albumin, or in Minimum Essential Medium Earle's Salt (MEME) containing 10% sorbitol. However, the chimeras can simply be diluted in a physiologically acceptable solution, such as sterile saline or sterile buffered saline. In addition, the chimeras can be mixed to form a tetravalent preparation at any point during formulation and administered together, or can be administered in series.

Optionally, the vaccines of the invention can include or be administered with an adjuvant or carrier, in addition to the live, attenuated chimeric viruses. In addition, cytokines (e.g., GM-CSF, IL-2, IL-12, IL-13, or IL-5) can be used, or genes encoding cytokines that have adjuvant activities can be inserted into the chimeric viruses.

The vaccines of the invention can be administered as primary prophylactic agents in adults or children at risk of Dengue infection, or can be used as secondary agents for treating Dengue-infected patients. Examples of patients who can be treated using the vaccines and methods of the invention include (i) children in areas in which Dengue is endemic, such as Asia, Latin America, and the Caribbean, (ii) foreign travelers, (iii) military personnel, and (iv) patients in areas of a Dengue epidemic. Moreover, inhabitants of regions into which the disease has been observed to be expanding (e.g., Argentina, Chile, Australia, parts of Africa, southern Europe, the Middle East, and the southern United States), or regions in which it may be observed to expand in the future (e.g., regions infested with Aedes aegypti), can be treated according to the invention.

The vaccines of the invention are administered using methods that are well known in the art. For example, the vaccines can be administered by subcutaneous, intramuscular, intradermal, or epidermal injection. In addition, the vaccines can be administered by mucosal (e.g., oral) routes.

Appropriate amounts of the vaccines to be administered to patients can readily be determined by those of skill in this art. Thus, the vectors of the invention can be formulated as sterile aqueous solutions containing between 10² and 10⁷ infectious units (e.g., plaque-forming units or tissue culture infectious doses) in a dose volume of 0.1 to 1.0 ml for administration. To reduce the possibility of viral interference and thus to achieve a balanced immune response, the amounts of each of the different chimeras present in the administered vaccines may not be equal. In particular, at least 5 fold less of one, two, or three chimeras (e.g., 10, 50, 100, 200, or 500 fold less) can be used relative to the other chimeras. In one example, the amounts of the Dengue-1, Dengue-2, Dengue-3, and Dengue-4 chimeras are equivalent (e.g., 5,5,5,5 or 4,4,4,4 log₁₀ PFU of each chimera). These amounts can vary and still be considered “equivalent.” For example, the amounts can vary by 10%, 25%, 50%, 75%, or even up to 100% and still be considered “equivalent.” In another example, the amounts of Dengue-3 and/or Dengue-4 virus can be decreased as well (e.g., 5,5,3,3 log₁₀ PFU of each chimera). For example, in addition to using less Dengue-2 chimera (e.g., 5,3,5,5 log₁₀ PFU of each chimera), at least 5 fold less of the Dengue-3 and Dengue-4 chimeras (e.g., 10, 50, 100, 200, or 500 fold less) can be used relative to the Dengue-1 chimera (e.g., 5,3,3,3). It may be particularly desirable, for example, to decrease the amount of Dengue-1 chimera relative to the amounts of Dengue-3 and/or Dengue-4 chimeras when the E204 mutation described above is not included in the Dengue-1 chimera (e.g., 3,5,5,5 log₁₀ PFU).

The vaccines of the invention can be administered in a single dose or, optionally, administration can involve the use of a priming dose followed by a booster dose that is administered, e.g., 2-6 months later, as determined to be appropriate by those of skill in the art.

One chimeric virus that can be used in the invention, which is a Yellow Fever virus 17D/dengue type 2 virus chimera, was deposited with the American Type Culture Collection (ATCC) in Manassas, Va., U.S.A. under the terms of the Budapest Treaty and granted a deposit date of Jan. 6, 1998 (YF/DEN-2; ATCC accession number ATCC VR-2593).

The invention is based, in part, on the following Experimental Results.

### Experimental Results

### Summary

Recombinant vaccines consisting of four DEN chimeras (ChimeriVax-DEN1 to DEN4) were developed using an infectious clone of Yellow Fever 17D virus, into which the envelope genes of wild type (WT) DEN viruses were inserted (Guirakhoo et al., Virology 75:7290-7304, 2001). These chimeras grew to high titers in Vero cells and were immunogenic in monkeys inoculated with monovalent or tetravalent (i.e., a mixture of equal concentrations of each monovalent chimeric virus) forms. However, it was noticed that in monkeys immunized with the tetravalent formulation, the highest immune response was directed toward the ChimeriVax-DEN2 virus. In vitro growth kinetic studies also revealed that ChimeriVax-DEN2 replicated more rapidly than the other 3 chimeras. Sequence analysis of chimeric DEN2 had revealed no mutations, but the DEN1, DEN3, and DEN4 chimeras contained a few mutations in the pre-membrane and envelope (prME) regions. These mutations had apparently been introduced during the construction of these chimeras, either intentionally upon introduction of new restriction sites, or unintentionally due to errors introduced by RT-PCR amplification of the prME genes of parental DEN viruses. To determine whether these mutations affected the growth rates of chimeric viruses in Vero cells or their safety and immunogenicity profiles in vivo, we reconstructed chimeric viruses without unnecessary mutations, and evaluated them for their in vitro growth kinetics, mouse neurovirulence, viremia, and immunogenicity in monkeys (Guirakhoo et al., Virology 298:146-159, 2002)

For production of vaccine viruses for human use, Pre Master Seed viruses (PMS) for chimeric viruses were produced by transfection of Vero LS-10 cell bank. Viruses were harvested from supernatants of infected cells (P1) and amplifed once to produce uncloned P2 PMS viruses. PMS (P2) viruses were biologically cloned by three rounds of direct plaque purification to produce PMS stock viruses at P7. These viruses were passaged three times under cGMP manufacture to produce Master Seed (P8), Working Seed (P9), and the Vaccine Bulks (P10) viruses. Additionally P10 viruses were produced by passaging the cGMP MS (P8) viruses in Vero LS-10 cells. These viruses (research P10 and cGPM P10) were evaluated by sequencing, mouse neurovirulence, safety tests in mosquito and monkey models, as well as immunogenicity and protective efficacy in monkeys. DEN1 and DEN2 chimeras acquired one and two mutations within the PrME-genes at P10 (vaccine level), respectively, whereas DEN3 and DEN4 chimeras maintained WT (wild type) PrME sequences throughout manufacturing. We found that chimeras which maintain the WT envelope sequences dominate those containing mutations (e.g., DEN2 without mutations; Guirakhoo et al., J. Virol., 75:7290-7304, 2001; DEN4 without mutations; Guirakhoo et al., Virology 298:146-159, 2002; and DEN1, DEN2 with mutations; see below), and therefore may need to be administered at a lower dose.

### Materials and Methods

### Reconstruction of ChimeriVax Dengue Viruses

Amino acid substitutions in the prME regions of the original ChimeriVax-DEN1, -DEN3, and -DEN4 constructs (designated as ChimeriVax-DEN1₉₉, -DEN3₉₉, and -DEN4₉₉) (Guirakhoo et al., Virology 75:7290-7304, 2001) were reverted to WT residues, and reconstructed viruses (designated as ChimeriVax-DEN 1₀₀, -DEN3₀₀, and -DEN4₀₀) were prepared by transfection of Vero cells with RNA transcripts as described previously (Guirakhoo et al., J. Virology 75:7290-7304, 2001; see below for details).

### Reconstruction of ChimeriVax-DEN™ and -DEN4 Viruses

Sequencing analysis of plasmids used for construction of these viruses revealed that some mutations were introduced due to errors in synthesis of primers. These primers were re-synthesized, new ChimeriVax-DEN1₀₀ and -DEN4₀₀ were constructed as described previously (Guirakhoo et al., J. Virology 75:7290-7304, 2001), and the new chimeras were sequenced across the prME regions (Table 1). An L to F mutation at amino acid E56 of ChimeriVax-DEN4₉₉ (nucleotide 666) (Table 1) was corrected by changing nucleotide T to C at adjacent nucleotide 664 (Guirakhoo et al., Virology 298:146-159, 2002).

### Reconstruction of ChimeriVax-DEN3 Virus

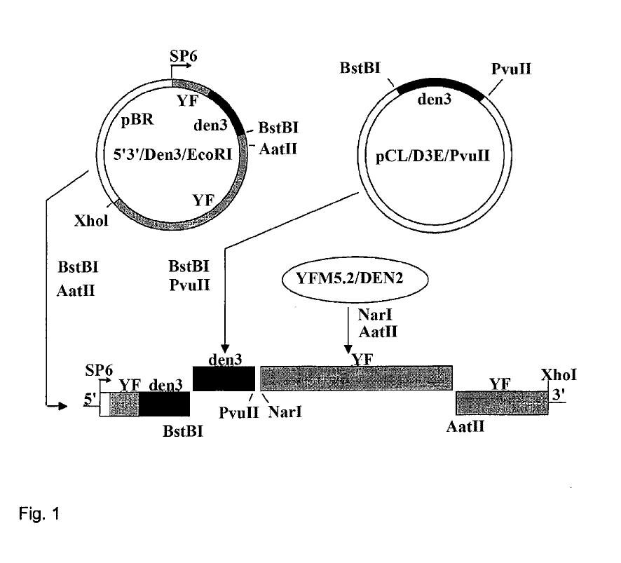

To eliminate amino acid substitutions in the envelope region of ChimeriVax-DEN3₉₉, as well as the majority of silent nucleotide changes including those introduced by using non-strain (DEN3 H87 virus) specific primers or intentionally engineered as restriction sites (e.g., the E492 mutation; Table 1; Guirakhoo et al., J. Virology 75:7290-7304, 2001), new oligonucleotides based on the sequence of the parent WT DEN3 (strain PaH881/88) were synthesized. These oligonucleotides were used to amplify the DEN3-specific region of 5′3′/Den3/ΔXhoI plasmid (FIG. 1) in which the mutations (except for the two silent nucleotide changes at nucleotide 9 (G to A) and 18 (C to A) of prM gene) were reversed. The corrected plasmid was designated 5′3′/Den3/EcoRI. The BstBI-NarI DEN3-specific part of plasmid 5.2/Den3 (Guirakhoo et al., J. Virology 75:7290-7304, 2001) was also amplified such that the NarI site was replaced with PvuII, and the PCR product was cloned in a modified low copy number vector pCL1921, resulting in plasmid pCL/D3E/PvuII. The reinserted parts of new plasmids were sequenced across both strands to ensure the absence of any PCR— or E. coli-induced mutations. To generate a DNA template for in vitro transcription, three-fragment ligation was performed as is shown in FIG. 1. The BstBI-PvuII fragment of pCL/D3E/PvuII and the Nar-AatII fragment of plasmid YFM5.2/DEN2 (containing YF-specific NS genes) were ligated with the large BstBI-AatII portion of 5′3′/Den3/EcoRI. Ligation products were linearized with XhoI and used for in vitro transcription with SP6 RNA polymerase. ChimeriVax-DEN3₀₀ virus was produced following transfection of Vero cells with the RNA transcripts. In contrast to the ChimeriVax-DEN3₉₉ (Guirakhoo et al., Virology 75:7290-7304, 2001), this new chimera was generated using only cDNA fragments derived from plasmids (without the PCR amplification step), thus eliminating the possibility of random PCR-induced nucleotide changes in the template. In addition, ligation between the PvuII and Nan blunt ends eliminated the E492 amino acid change at the E/NS1 junction. Thus, the new virus contained the authentic PaH881/88-specific signal for NS1 (Guirakhoo et al., Virology 298:146-159, 2002).

### Cells and Viruses

Vero cells used to produce and assay ChimeriVax-DEN viruses were obtained from Aventis Pasteur (Lyon, France). They were used between passages 141 and 151 for transfection of chimeric viruses, and between passages 143 and 170 for other assays, such as plaque assays and neutralization tests. For cGMP vaccine production

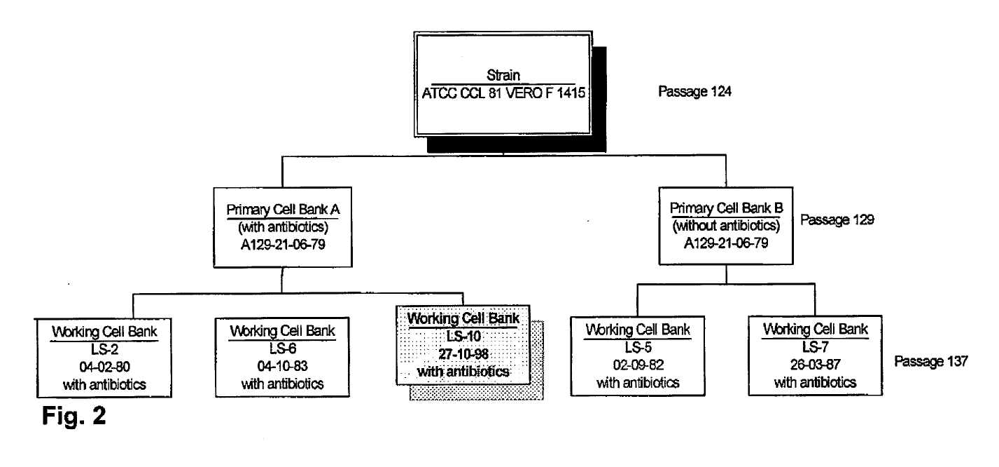

Vero cells were obtained from a qualified working cell bank from Aventis Pasteur (France), and grown in MEME containing 10% FBS (from herds in non-BSE countries, obtained from Hyclone). The passage history of production of these cells is shown in FIG. 2. LS-10 cell bank (P137) was used in the production of PMS (non-cGMP), MS (cGMP), WS (cGMP), and the Bulk product (BP) viruses. C6/36 cells used to grow WT DEN viruses were obtained from the American Type Culture Collection (ATCC, Manassas, Va.). WT DEN1 viruses used were strains PUO-359, Thailand 1980; BE H 455823, Brazil 1986; and 85-464, Indonesia 1985. WT DEN2 viruses used were strains PUO-218, Thailand 1980; S16803, Thailand 1974; JAH, Jamaica 1982; and PR 159, Puerto Rico 1969. WTDEN3 viruses were strains PaH881/88, Thailand 1988; 1301, Malaysia 1975; and 1325, Sri Lanka 1981. WT DEN4 viruses used were strains 1228, Indonesia 1978; BC 26-97, Mexico 1996; and P75-215, Malaysia 1975. These viruses, which were selected from a library of low-passage WT dengue viruses based on geographic origin and putative genotypic differences, are all representative of the human-A edes aegypti dengue virus transmission cycle, with the exception of DEN3 P75-215. This virus is considered to be a “sylvatic” strain, because it was isolated from canopy-dwelling mosquitoes (Ae. niveus) (Wang et al., Virology 74:3227-3234, 2000) (Table 2). Chimeric viruses used in these studies were ChimeriVax-DEN1₉₉(VeroP4), ChimeriVax-DEN2 (VeroP3), ChimeriVax-DEN3₉₉ (VeroP4), ChimeriVax-DEN3₀₀ (VeroP5), ChimeriVax-DEN4₉₉ (three-times plaque purified large and small plaque variants, VeroP8), and ChimeriVax-DEN4₀₀ (VeroP5) (Table 1). Commercial YF 17D vaccine (YF-VAX®) was purchased from Aventis Pasteur (Lyon, France), and was used unpassaged.

### Production of PMS for ChimeriVax™-DEN1-4 Viruses

ChimeriVax™-DEN2 and ChimeriVax™-DEN4 PMS viruses were produced using the standard two-plasmid method previously utilized to create ChimeriVax™-JE (Guirakhoo et al., Virology 257:363-372, 1999) and ChimeriVax™-DEN2 (Guirakhoo et al., J. Virology 74:5477-5485, 2000) vaccine viruses for which INDs have been submitted and approved (BB-IND #9167 and BB-IND #10211, respectively).

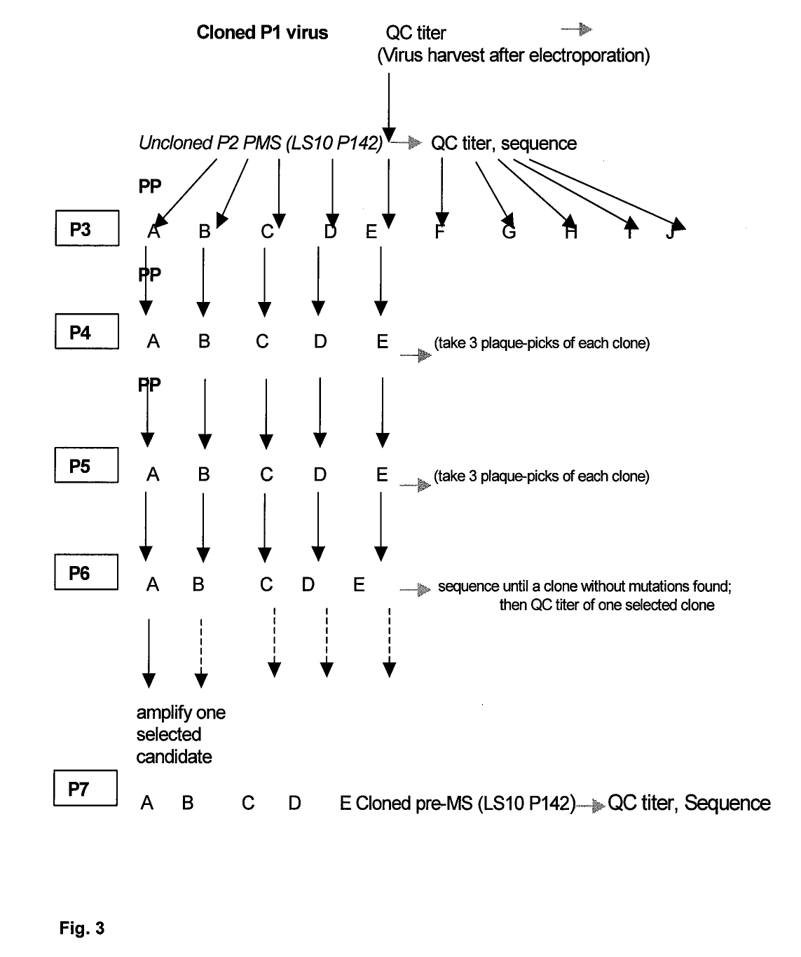

The essence of the two-plasmid approach is the cloning of the chimeric genome in two plasmids and regeneration by in vitro ligation of two appropriate plasmid DNA fragments, followed by in vitro transcription and transfection of cells with the RNA transcripts. ChimeriVax™-DEN1 and ChimeriVax™-DEN3 PMS viruses were produced using a novel three-plasmid method. First, the chimeric genome is stably cloned in three plasmids, then reproduced by ligation of three appropriate DNA fragments excised from the three plasmids, followed by in vitro transcription and transfection of cells with synthesized RNA transcripts. All described PMS viruses were produced in Vero LS-10 cells. The production strategy of the viruses is shown in FIG. 3. Primary PMS candidates were the three-times plaque-purified (cloned) viruses, which were first selected based on their full-genome sequence, i.e., shown to be free from amino acid substitutions at the P6 and the final P7 (PMS) levels. The selected cloned PMS virus candidates were further passaged in Vero LS-10 cells to ascertain their genetic stability in cell culture and tested in animal models for safety and immunogenicity at appropriate passages.

### Production of ChimeriVax™-DEN1 Pre-Master Seed (P7)

I. Preparation of Chimeric YF/DEN1 RNA

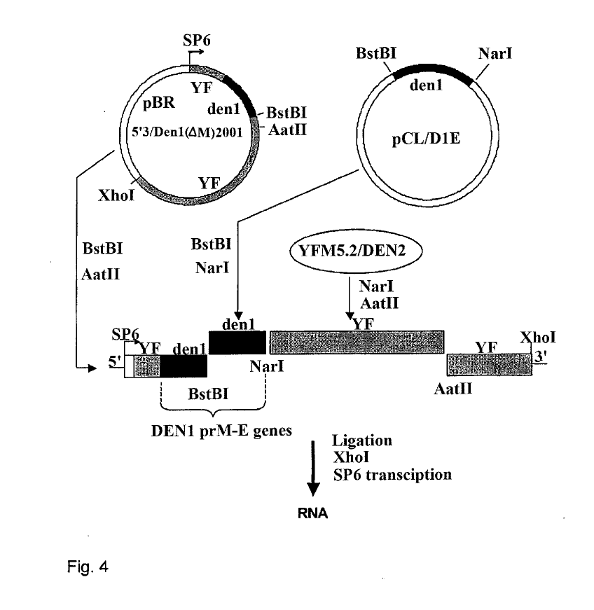

To produce YF/DEN1 RNA, the three-plasmid strategy was employed in the same way as for YF/DEN3 described below. To synthesize in vitro RNA transcripts used for the PMS production of ChimeriVax™-DEN1 (FIG. 4) plasmids 5′3′DEN1(ΔM)2001 (clone 7), pCL/DEN1E (clone 16), and YFM5.2/DEN2 (the third plasmid used as a source of YF-specific nonstructural protein genes; clone 3.4) were propagated in Luria broth (LB) or Terrific broth (TB; Gibco) media, and purified using Qiagen-100 columns (Qiagen). The DNAs were resuspended in elution buffer (EB), and their concentrations were measured using a spectrophotometer at 260 nm. Ten μg of each plasmid was subjected to digestion with appropriate restriction endonucleases as follows: The 5′3′DEN1(ΔM)2001 plasmid was digested with BstBI and AatII, the pCL/DEN1E plasmid was digested with BstBI and NarI, and the YFM5.2/DEN2 plasmid was cut with NarI and AatII. The digestion products were separated in a 0.8% agrose gel, and appropriate fragments (FIG. 4) were eluted from the gel using QIAquick Gel Extraction Kit (Qiagen). Five hundred (500) ng of the 5.6 kb fragment from the 5′3′DEN1(ΔM)2001 plasmid, 200 ng of the 1.3 kb fragment from the pCL/DEN1E plasmid, and 500 ng of the 5.95-kb fragment from the YFM5.2/DEN2 plasmid was ligated using T4 DNA ligase overnight at 16° C. The ligated DNA was then digested with XhoI to allow for run-off transcription and phenol-chloroform extracted, after which the full-length cDNA was transcribed in vitro with SP6 RNA polymerase to produce RNA for transfection. A full-length YF/DEN1 RNA band was detectable in a 2 μl aliquot of the reaction mixture in an agarose gel (estimated full-length RNA concentration ˜10 ng/μl). Two aliquots (18 μl/aliquot) of the RNA transcripts were stored at ≦−60° C.

II. Transfection of Vero Cells with Chimeric YF/DEN1 RNA

Vero LS-10 cells at passage 140 were thawed and propagated to passage 144 in T-150 cm² flasks. Cells were trypsinized, washed with PBS, and electroporated with one aliquot (18 μl) of the chimeric YF/DEN1 RNA transcripts by one pulse at 320 V and 950 μF: After electroporation, cells were transferred to a T-75 cm² flask containing 25 ml EMEM, 5% FBS (Hyclone) and neomycin, and incubated at 37° C., 5% CO₂. On Day 3, when the cytopathic effect (CPE) was ˜30%, the culture fluid (P1 virus) was harvested, clarified by low-speed centrifugation (1200 rpm, 5 minutes, 4° C.), 0.22 μm filtered, supplemented with FBS (50% final concentration), aliquoted and frozen at ≦−60° C. Titer of the P1 virus determined by plaque assay in Vero cells was 4.4×10⁵ PFU/ml.

III. Preparation of ChimeriVax™-DEN 1 Cloned PMS

A cloned DEN 1 PMS virus was produced as follows:

Vero LS-10 cells at passage P142 were inoculated with YF/DEN1 P1 virus at an MOI of 0.001 PFU/cell. Virus-containing supernatants were harvested on Day 4 when ˜5% CPE was observed. The titer of Uncloned P2 determined by plaque assay in Vero cells was 2.9×10⁶ PFU/ml.

The P2 virus was plagued in Vero LS-10 cells, and ten well-isolated plaques (designated plaques A through J; virus passage P3) were isolated using a sterile glass Pasteur pipette. Each harvested plaque was placed in 0.4 ml of M199 medium containing 50% FBS, and frozen at −80° C. The P3 plaque J was subjected to two additional rounds of direct plaque purification (virus passages P4 and P5) without any intermediate virus amplification steps. The 3× plaque-purified Clone J virus (P5) was amplified in a T-25 cm² flask of Vero LS-10 cells. The P6 Clone J virus was harvested on Day 3 (˜5% CPE), clarified, supplemented with FBS to 50% concentration, aliquoted, and stored at −80° C. Its titer was determined to be 9.0×10⁵ PFU/ml. To produce the final P7 Clone J PMS, three T-150 cm² flasks of Vero LS-10 cells expanded to passage P142 and were infected with the P6 clone J virus at an MOI of 0.001 PFU/cell. Fifty ml/flask of maintenance medium (EMEM, 10% FBS) was added, and flasks were incubated at 37° C., 5% CO₂. Virus-containing supernatants were harvested on Day 3 (˜7% CPE) and clarified by low-speed centrifugation (1200 rpm, 10 minutes, 4° C.). After removal of aliquots for QC testing, clarified supernatants were filtered through a 0.22 μm filter, and FBS was then added to the final concentration of 50%. Clone J P7 PMS virus was aliquoted and frozen at −80° C. The titer, as determined by plaque assay in Vero cells, was 3.3×10⁶ PFU/ml.

### Production of ChimeriVax™-DEN2 Pre-Master Seed (P7)

I. Preparation of Chimeric YF/DEN2 RNA

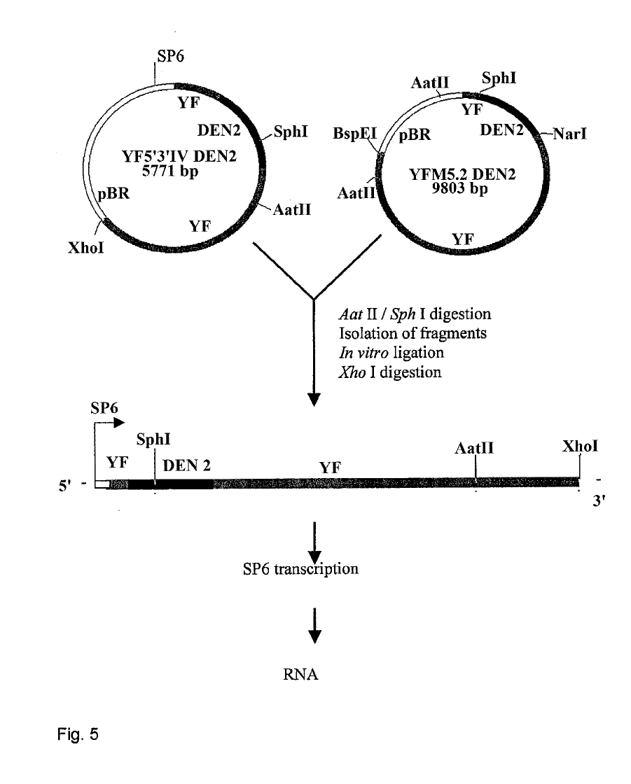

Development of the ChimeriVax™-DEN2 vaccine began by cloning a cDNA copy of the entire 11-kilobase (kb) genome of YF 17D virus. To accomplish this, YF 17D genomic sequences were propagated in two plasmids, which encode the YF sequences from nucleotides (nt) 1-2276 and 8279-10,861 (plasmid YF5′3′IV), and from 1373-8704 (plasmid YFM5.2), respectively. Full-length cDNA templates were generated by ligation of appropriate restriction fragments derived from these plasmids. The YF-specific prM-E genes within the YF 5′3′IV and YFM5.2 plasmids were replaced by the corresponding DEN2 (strain PUO 218) prM-E sequences, resulting in the generation of YF5′3′IV/DEN2 (contains the DEN2-specific prM gene and the 5′ portion of E) and YFM5.2/DEN2 (containing the 3′ portion of DEN2 E gene) plasmids. To synthesize in vitro RNA transcripts used for the PMS production, plasmids YF5′3′IV/DEN2 (clone 5) and YFM5.2/DEN2 (clone 3.4) were propagated in Luria broth (LB) medium, and purified using Qiagen-100 columns (Qiagen). The DNAs were resuspended in 200 and 100 μl of elution buffer (EB), respectively, and their concentrations were measured using a spectrophotometer at 260 nm. DNA concentrations of these samples were 445 ng/μl and 452 ng/μl for YF5′3′IV/DEN2 and YFM5.2/DEN2 plasmids, respectively. Ten μg of each plasmid was subjected to double digestion with SphI and AatII restriction endonucleases at 37° C. for 2 hours in a water bath. The digestion products were separated in 0.8% agarose gel, and appropriate fragments (the largest, ˜6 kb, from each of the two digests) were eluted from the gel using QIAquick Gel Extraction Kit (Qiagen). Five hundred ng of the YF5′3′IV/DEN2-fragment and five hundred) ng of the YFM5.2/DEN2-fragment were ligated using T4 DNA ligase overnight at 16° C. The ligated DNA was then digested with XhoI enzyme to allow run-off transcription and phenol-chloroform extracted, after which the full-length cDNA was transcribed in vitro with SP6 RNA polymerase to produce RNA for transfection (FIG. 5). The yield of YF/DEN2 RNA estimated on the gel was 50-100 ng/μl. Three aliquots (6 μl/aliquot) were stored at −60° C.

II. Transfection of Vero Cells with Chimeric YF/DEN2 RNA

Vero LS-10 cells at passage 140 were thawed and grown in two T-75 cm² flasks in EMEM containing 10% FBS (Hyclone), and then further expanded in T-150 cm² flasks. Cells at passage 142 were electroporated with the chimeric YF/DEN2 RNA transcripts (˜300 ng) at 320 V and 950 μF, one pulse. After electroporation, cells were transferred to a T-75 cm² flask containing 25 ml EMEM, 5% FBS (Hyclone) and neomycin, and incubated at 37° C., 5% CO₂. After 24 hours, medium was changed. On Day 3 (15% CPE), the culture fluid (P1 virus) was harvested, clarified by low-speed centrifugation (1200 rpm, 5 minutes, 4° C.), 0.22 μm filtered, supplemented with FBS (50% final concentration), aliquoted, and frozen at ≦−60° C. The titer of P1 determined in a plaque assay in Vero cells was 4.7×10⁶ PFU/ml.

III. Preparation of ChimeriVax™-DEN 2 Cloned PMS

A cloned DEN 2 PMS virus was produced as follows:

Vero LS-10 cells at passage P142 were inoculated with YF/DEN2 P1 virus at an MOI of ˜0.001 PFU/cell. Virus-containing supernatants were harvested on Day 4 when 10% CPE was observed. The titer of Uncloned P2 determined by plaque assay in Vero cells was 9.0×10⁶ PFU/ml.

The P2 virus was plagued in Vero LS-10 cells, and ten well-isolated plaques (designated plaques A through J; virus passage P3) were isolated using a sterile glass Pasteur pipette. Each harvested plaque was placed in 0.4 ml of M199 medium containing 50% FBS, and frozen at −80° C. The P3 plaque A was subjected to two additional rounds of direct plaque purification (virus passages P4 and P5) without any intermediate virus amplification steps. The 3× plaque-purified Clone A virus (P5) was amplified in a T-25 cm² flask of Vero LS-10 cells, and the P6 Clone A virus was harvested on Day 3 (10% CPE), supplemented with 50% FBS, and stored at −80° C.; its titer was determined to be 1.8×10⁷ PFU/ml. To produce the final P7 Clone A PMS, three T-150 cm² flasks of Vero LS-10 cells expanded to passage P142, were infected with the P6 clone A virus at an MOI of 0.001 PFU/cell. Fifty ml/flask of maintenance medium (EMEM, 10% FBS) was added, and flasks were incubated at 37° C., 5% CO₂. Virus-containing supernatants were harvested on Day 4.5 (˜10% CPE) and clarified by low-speed centrifugation (1200 rpm, 10 minutes, 4° C.). After removal of aliquots for QC testing, clarified supernatants were filtered through a 0.22 μM filter (Corning), and FBS was then added to the final concentration of 50%. The Clone A P7 PMS virus was aliquoted and frozen at −80° C. The titer of this virus, determined by plaque assay in Vero cells, was 9.7×10⁶ PFU/ml.

### Preparation of Chimeric YF/DEN3 Pre-Master Seed (P7)

I. Preparation of Chimeric YF/DEN3 RNA

Initial attempts to construct a stable DEN3-specific analog of YFM5.2 plasmid (such as YFM5.2/DEN2 plasmid described above) were not successful, probably due to plasmid toxicity for E. coli. Thus, the three plasmid cloning strategy to create the ChimeriVax™-DEN3 vaccine was applied. DEN3 (strain PaH881/88) prM-E sequences were used to construct necessary plasmids. First, plasmid 5′3′DEN3/EcoRI was constructed, which is an analog of the YF5′3′IV series of plasmids (such as YF5′3′IV/DEN2) containing the DEN3-specific prM gene and the 5′ portion of E. Second, the 3′ portion of the DEN3 E gene normally present in YFM5.2 plasmids (e.g., YFM5.2/DEN2; see FIG. 5) was individually cloned in a stable pCL/DEN3E/PvuII plasmid. To synthesize in vitro RNA transcripts used for the PMS production plasmids 5′3′DEN3/EcoRI (clone 1), pCL/DEN3E/PvuII (clone 18) and YFM5.2/DEN2 (the third plasmid used as a source of YF-specific nonstructural protein genes; clone 3.4) were propagated in Luria broth (LB) or Terrific broth (TB; Gibco) media, and purified using Qiagen-100 columns (Qiagen). The DNAs were resuspended in 200 μl and 100 μl of elution buffer (EB), respectively, and their concentrations were measured at 260 nm using a spectrophotometer. Ten μg of each plasmid was subjected to digestion with appropriate restriction endonucleases as follows: The 5′3′DEN3/EcoRI plasmid was digested with BstBI and AatII, the pCL/DEN3E/PvuII plasmid was digested with BstBI and PvuII, and the YFM5.2/DEN2 plasmid was cut with NarI and AatII. The digestion products were separated in a 0.8% agarose gel, and appropriate fragments (FIG. 1) were eluted from the gel using QIAquick Gel Extraction Kit (Qiagen). Five hundred ng of the 5654 basepair fragment from the 5′3′DEN3/EcoRI plasmid, 200 ng of the 1307-basepair fragment from the pCL/DEN3E/PvuII plasmid, and 500 ng of the 5955-bp fragment from the YFM5.2/DEN2 plasmid was ligated overnight at 16° C. using T4 DNA ligase (FIG. 1). The ligated DNA was then digested with XhoI to allow for run-off transcription and phenol-chloroform extracted, after which the full-length cDNA was transcribed in vitro with SP6 RNA polymerase to produce RNA for transfection. A full-length of YF/DEN3 RNA band was detectable (using 2-μl aliquot of the reaction mixture) in an agarose gel. Two aliquots (9 μl/aliquot) of the RNA transcripts were stored at ≦−60° C.

II. Transfection of Vero Cells with Chimeric YF/DEN3 RNA

Vero LS-10 cells obtained from Aventis Pasteur at passage 137 were thawed and propagated to passage 142 in T-150 cm² flasks. At passage 142, cells were electroporated with one aliquot (9 μl) of the chimeric YF/DEN3 RNA transcripts by one pulse at 320 V and 950 μF. After electroporation, cells were seeded in a T-75 cm² flask containing 25 ml EMEM, 5% FBS (Hyclone) and neomycin, and incubated at 37° C., 5% CO₂. On Day 3, when CPE was ˜20%, the culture fluid (P1 virus) was harvested, clarified by low-speed centrifugation (1200 rpm, 5 minutes, 4° C.), 0.22 μm filtered, supplemented with FBS (50% final concentration), aliquoted, and frozen at −60° C. The titer of P1 virus determined by plaque assay in Vero cells was 1.8×10⁶ PFU/ml.

III. Preparation of ChimeriVax™-DEN3 Cloned PMS

A cloned DEN 3 PMS virus was produced as follows:

Vero LS-10 cells, passage 140 were grown and expanded to passage 142 (in T-150 cm² flasks). At passage 142, the cells were inoculated with YF/DEN3 P1 at an MOI of ˜0.001 PFU/cell. Virus-containing supernatants were harvested on Day 5, when ˜5% CPE was observed. The titer of Uncloned P2 determined by plaque assay in Vero cells was 2.1×10⁶ PFU/ml.

The P2 virus was plagued in Vero LS-10 cells, and ten well-isolated plaques (designated plaques A through J viruses, passage P3) were isolated using a sterile glass Pasteur pipette. Each harvested plaque was placed in 0.4 ml of M199 medium containing 50% FBS, and kept frozen at −80° C. P3 plaque A was subjected to two additional rounds of direct plaque purification (virus passages P4 and P5) without any intermediate virus amplification steps. The 3× plaque-purified Clone A virus (P5) was amplified in a T-25 cm² flask of Vero LS-10 cells, and P6 Clone A virus was harvested on Day 3 (˜3% CPE), clarified, supplemented with FBS to 50% concentration, aliquoted, and stored at −80° C.; its titer was determined to be 1.4×10⁶ PFU/ml. To produce the final P7 Clone A PMS, three T-150 cm² flasks of Vero LS-10 cells expanded to passage P142 were infected with P6 clone A virus at an MOI of 0.001 PFU/cell. Fifty ml/flask of maintenance medium (MEME, 10% FBS) was added, and flasks were incubated at 37° C., 5% CO₂. Virus-containing supernatants were harvested on Day 4 (˜10% CPE) and clarified by low-speed centrifugation (1200 rpm, 10 minutes, 4° C.). After removal of aliquots for QC testing, clarified supernatants were filtered through a 0.22-μm filter, and FBS was then added to the final concentration of 50%. Clone A P7 PMS virus was aliquoted and frozen at −80° C. The titer of Clone A P7 PMS determined by plaque assay in Vero cells was 1.2×10⁶ PFU/ml.

### Production of ChimeriVax™ DEN4 Pre-Master Seed (P7)

I. Preparation of Chimeric YF/DEN4 RNA

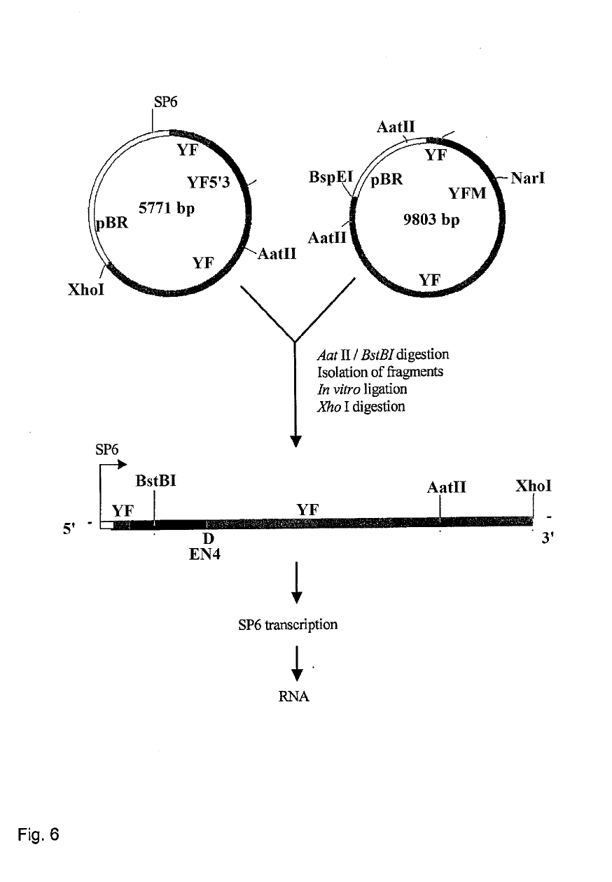

To produce YF/DEN4 RNA, the two-plasmid strategy was used, as described for YF/DEN2 (FIG. 6). Two necessary plasmids, YF5′3′IV/DEN4 and YFM5.2/DEN4, were constructed containing the prM-E sequences from DEN4 virus, strain 1228. The overall structure of these plasmids is essentially the same as shown in FIG. 5 for YF/DEN2 plasmids, with only one exception: in YF/DEN4 plasmid, the BstBI restriction site was used instead of SphI. This site was necessary for in vitro ligation to generate the full-length YF/DEN4 cDNA template. To synthesize in vitro RNA transcripts used for ChimeriVax™-DEN4 PMS production, the two plasmids, YF5′3′IV/DEN4 (Clone R2) and YFM5.2/DEN4 (Clone 4), were propagated in 150 ml LB medium, and purified using Qiagen-100 columns (Qiagen). The DNAs were resuspended in 200 μl and 100 μl of elution buffer (EB), respectively, and their concentrations were measured at 260 nm using a spectrophotometer. Ten μg of each plasmid was digested with BstBI and AatII restriction endonucleases. The digestion products were separated in 0.8% agarose gel, and appropriate fragments (the largest, ˜6 kb, from each of the two digests) was eluted from the gel using QIAquick Gel Extraction Kit (Qiagen). Three hundred ng of the YF5′3′IV/DEN4-fragment and 130 ng the YFM5.2/DEN4-fragment was ligated overnight at 16° C. using T4 DNA ligase. The ligated DNA was then digested with XhoI enzyme to allow run-off transcription, phenol-chloroform extracted, and the full-length cDNA was transcribed in vitro with SP6 RNA polymerase to produce RNA for transfection. The yield of YF/DEN4 RNA estimated on the gel was 80 ng/μl. Three aliquots (5 μl/aliquot) were stored at ≦−60° C.

II. Transfection of Vero Cells with Chimeric YF/DEN4 RNA

Vero LS-10 cells at passage 140 were propagated to passage 142. Cells at passage 142 grown in T-150 cm² flasks were electroporated with the chimeric YF/DEN4 RNA transcripts (Sample D; 5 ul, ˜400 ng) by one pulse at 320 V and 950 μF. After electroporation, cells were seeded in a T-75 cm² flask containing EMEM, 5% FBS (Hyclone) and neomycin, and incubated at 37° C., 5% CO₂. After 24 hours, medium was changed with 25 ml of fresh medium. On Day 4 (˜10% CPE), the culture fluid (P1 virus) was harvested, clarified by low-speed centrifugation (1200 rpm, 5 minutes, 4° C.), 0.22 μm filtered, supplemented with FBS (50% final concentration), aliquoted, and frozen at ≦−60° C. The titer of P1 virus determined in a plaque assay in Vero cells was 1.5×10⁶ PFU/ml.

III. Preparation of ChimeriVax™-DEN4 PMS (P7) (Non-GMP)

A cloned DEN4 PMS virus was produced as follows:

Vero LS-10 cells were propagated to passage P142. Three T-150 cm² flasks of these cells were inoculated with the YF/DEN4 P1 virus at an MOI of ˜0.001 PFU/cell. Virus-containing supernatants were harvested on Day 5, when ˜10% CPE was observed. The virus (Uncloned P2 PMS) was aliquoted and frozen at ˜80° C. The titer of Uncloned P2 PMS candidate determined by plaque assay in Vero cells was 2.2×10⁷ PFU/ml.

The P2 virus was plagued in Vero LS-10 cells, and ten well-isolated plaques (designated plaques A through J; virus passage P3) were isolated using sterile glass Pasteur pipette. Each harvested plaque was placed in 0.4 ml of M199 medium containing 50% FBS and frozen at −80° C. P3 plaque B was subjected to two additional rounds of direct plaque purification (virus passages P4 and P5) without any intermediate virus amplification steps. The 3× plaque-purified Clone B virus (P5) was amplified in a T-25 cm² flask of Vero LS-10 cells to produce P6 virus, which was harvested on Day 3 (10% CPE). P6 virus was supplemented with 50% FBS, and stored at −80° C. Its titer was determined to be 2.1×10⁶ PFU/ml. To produce the final P7 Clone B PMS, three (3) T-150 cm² flasks of Vero LS-10 cells expanded to passage P142, were infected with P6 clone B virus at an MOI of 0.0046 PFU/cell. Fifty ml/flask of maintenance medium (EMEM, 10% FBS) was added, and flasks were incubated at 37° C., 5% CO₂. Virus-containing supernatants were harvested on Day 4 (˜10% CPE) and clarified by low-speed centrifugation (1200 rpm, 10 minutes, 4° C.). After removal of aliquots for QC testing, clarified supernatants were filtered through a 0.22 μm filter (Corning), and FBS was then added to the final concentration of 50%. Clone B P7 PMS virus was aliquoted and frozen at −80° C. The titer of this virus, determined by plaque assay in Vero cells, was 2.5×10⁶ PFU/ml.

### Nucleotide Sequencing of ChimeriVax™-DEN Viruses

For sequencing, viral RNA was extracted from each indicated virus sample (generally 250 μl) using TR1—Reagent LS (Molecular Research Center) or Trizol LS (a similar reagent from Gibco) and dissolved in 20 μL of RNase-free water. The extracted RNA was then used as a template for RT-PCR. The entire genome was amplified in five overlapping amplicons of ˜2-3 kb in length (fragments I through V) with the Titan One-Tube RT-PCR kit (Roche). The RT-PCR fragments were purified using QIAquick PCR Purification kit (Qiagen) or agarose gel-purified using QIAquick Gel Extraction kit (Qiagen). Sequencing reactions were done using CEQ Dye Terminator Cycle Sequencing kit (Beckman), and a collection of dengue-specific and YF-specific oligonucleotide primers of both positive and negative orientation to read both strands of the amplicons. Sequencing reaction products were purified using DyeEx Spin kit (Qiagen), and resolved with a CEQ2000 automated sequencer (Beckman Coulter). Generated sequencing data were aligned and analyzed with Sequencher 3.0 (GeneCodes) software. Nucleotide heterogeneities were registered only when a heterogeneous signal was observed in all chromatograms representing both plus- and minus-strand sequencing reactions.

All PMS seeds of ChimeriVax-DEN1-4 viruses at P7 were free from any amino acid substitutions in their entire genome. ChimeriVax™-DEN3 contained one silent nucleotide change C>T at NS4a 6607.

Preparation of Master Seed (P8) seed of Chimeri Vax™ DEN1-4

The Master Seed viruses were prepared at Molecular Medicine (La Jolla, Calif.,) under cGMP.

Vero cells were allowed to grow to near confluence in a 10-layer NCF, after which they were infected with virus. Virus-containing cell culture supernatant fluids were harvested and processed by filtration to remove cell debris.

Viral materials from the pre-master seeds were used in the preparation of MS. One vial of Vero cells (LS-10, Aventis Pasteur) was thawed, and cells were seeded at a density of approximately 3-5×10⁴ cells/cm² into 5-12 T225 cm² flasks. The biomass expansion was carried out in these T-flasks, until there were a sufficient number of cells available to seed a 10-layer NCF.

Growth medium consists of MEME with L-glutamine supplemented with 2 mM L-glutamine+1% MEM Non-essential AA+10% 0.1 μm filtered, gamma irradiated FBS.

The infected NCF and flasks were incubated at 36±2° C., 5±2% CO₂, and 80±5% relative humidity for 3-4 days. The NCF were infected with PMS Viruses (P7 of either of 4 ChimeriVax™-DEN viruses) at an MOI of approximately 0.001 by adding MEME with L-glutamine supplemented with 2 mM L-glutamine+1% MEM Non-essential AA+10% 0.1 μm filtered, gamma irradiated FBS. The NCF were harvested when early CPE was observed in infected NCF2 and T-225 flasks by comparing with un-inoculated flasks as a reference control. The NCF were removed from the incubator, and the infected culture medium containing the MS viruses (passage 8) were aseptically harvested into sterile containers. After sampling for QC tests, the remaining bulk MS materials were formulated by adding 0.1 μm filtered, gamma irradiated FBS to the Bulk Harvest material to a final concentration of 50%, and formulated materials were filtered through a sterile 0.22 μm filter.

The MS (P8) viruses were filled in 10 ml aliquots into 30 ml sterile PETG bottles, and were stored at ≦−60° C. or below.

### Preparation of Production/Working Seed (P9) for ChimeriVax™ DEN1-4 Viruses

The Working Seed Viruses were produced under cGMP by Aventis-Pasteur (Marcy L'Etoile, France). Passage 9, WS (P9) is manufactured, formulated, and filled in the same manner as MS (P8): Vero cells are expanded into NCF and P8 MS viruses to infect at an MOI of 0.001 PFU/cell. For manufacture of WS, Vero cells from the LS-10 bank (Aventis-Pasteur) are used. One or 2 vials of Vero cells at 139^{th} passage issued from the LS10 Working Cell Bank at 137^{th} passage were thawed in water bath at 30° C. Cells were grown with culture medium A (MEME with 2 mM L-glutamine, 1% non-essential amino acids and 10% heat-treated FCS, 150 ml/T-225) in 3 T-225 flasks at 37±1° C. and 5% CO₂.

The cell expansion is performed by two subcultures at 7 days interval. The cells were expanded into new culture flasks or NCFs at initial concentration of 30,000-40,000 cells/cm². At the last subculture (142^{nd} passage), 10 vials of 10⁷ cells/vial were prepared and stored in liquid nitrogen for archive. The remaining cells were seeded in 25 F150 (25×150 cm²) for control cells and 2 NCFs (2×6000 cm₂) for production.

The control cells were examined for 19 days, then underwent Quality Control testing (hemadsorption, sterility, mycoplasma, adventitious virus on indicator cells, and identity tests).

The cells for production were grown with culture medium A containing 10% heat-treated FCS. After 5 days incubation at 37±1° C., the NCFs were inoculated with P8 viruses (either of DEN1-4 viruses) at an MOI of 0.001. After ˜4 days incubation at 37° C., the culture supernatants containing viruses were harvested. Samples of viral harvests were taken for quality controls (sterility test, mycoplasma, mycobacterium, and adventitious virus on indicator cells and suckling mice), the viral harvests (2×3000 ml) were then diluted at ½ with heat-treated FCS. The diluted viral solutions were 0.2 μm filtered, and samples were taken for Quality Control tests (identity test and virus concentration). The WS lot (6 L for each serotypes) was filled in sterile polypropylene bottles containing different volumes (5-20-100-500 ml) and stored at 5-35° C.

### Vaccine Production (P10)

P10 bulk purified vaccine was manufactured under cGMP by Aventis Pasteur.

As for manufacture of seed viruses, the process involved a biomass expansion phase in which the numbers of Vero cells (LS-10, Aventis-Pasteur) were expanded using stationary culture flasks and NCFs. At the virus infection phase there were wash steps (2× serum free medium) prior to virus infection and a re-feed with serum-free medium, both of which are were designed to reduce the levels of FBS in the final bulk. Downstream processing consisted of filtration to remove cell debris followed by digestion of nucleic acids by Benzonase®, concentration of the virus and final filtration.

Vero cells grown in NCFs were infected with WS (P9) virus at an MOI of 0.001. The spent cell culture medium was removed from the NCFs, and each NCF was rinsed twice with at least 250 ml of MEME with 2 mM L-glutamine, without phenol red or FBS. WS virus (P9) diluted in a small volume of MEME with 2 mM L-glutamine, without phenol red or FBS was then pumped into the NCFs and allowed to adsorb for 1 hour. After the 1 hour adsorption, MEME with 2 mM L-glutamine without phenol red or FBS was aseptically added, and the infected NCFs were incubated at 36±2° C., 5±2% CO₂ and 80±5% relative humidity for 3-4 days.

At the time of harvest, the NCFs culture fluid containing the P10 virus was aseptically harvested (2×3000 ml) and samples for Quality Control removed before 0.2 μm filtration. The downstream processing consisted of digestion of nucleic acids by Benzonase® treatment (15 UI/ml at 5±3° C. for 16 hours), followed by 0.2 μm filtration. The filtrate was then concentrated (10 folds) and diafiltered (50 kD) against MEME without phenol red, FCS. A sample is taken for Benzonase® and residual DNA testing after 0.2 μm filtration. Finally, the filtered concentrate was stabilized by adding 40% sorbitol solution (3 volumes concentrate plus 1 volume of stabilizer) to reach a final sorbitol concentration of 10%. The stabilized concentrate constituted the formulated Bulk Product (800 ml).

Bulk purified vaccine was stored at ≦35° C. before shipping to Aventis Pasteur (Swiftwater, Pa.) for filling.

### Animal Studies

All studies were carried out under an Institutional Animal Care and Use Committee (IACUC) approved protocol in accordance with the USDA Animal Welfare Act (9 C.F.R. Parts 1-3), as described in the Guide for Care and Use of Laboratory Animals (NIH, 1998).

I. Mice.

Neurovirulence of viruses was determined in suckling mice born on site from pregnant ICR mice (Taconic Farm, Inc., Germantown, N.Y.). Mice were pooled at 3 days of age to reduce genetic variations within the assay, randomly redistributed to mothers (˜10 sucklings/mother), and inoculated at different days of age with 20 μl of virus suspension by the intracerebral (i.c.) route. Animals were observed for 21 days, and mortality recorded. Any animal found in an advanced moribund stage was euthanized according to the IACUC protocol. Mortality ratios by dose, 50% lethal dose (LD₅₀) values, were calculated by the Reed and Muench method (1938).

II. Monkeys.

Six experiments were conducted in monkeys under GLP at Sierra Biomedical (Nevada, USA) to assess the safety and viscerotropism/immunogenicity of various passages of ChimeriVax™-DEN1-4 chimeras inoculated by the subcutaneous route (SC) (Experiments 1-4) and toxicity (experiments 5-6) by the intracerebral inoculation (IC) as prescribed by the WHO requirements for preclinical neurovirulence studies of yellow fever vaccines (WHO Technical Report Series, No. 872, 1998).

The studies were as follows:
1. Viremia and immunogenicity of reconstructed ChimeriVax™-DEN1-4 viruses
2. A 31-Day Comparative Immunogenicity Study of Three YF/DEN-1 Vaccines Administered by a Single Subcutaneous Injection to Rhesus Monkeys

3. A 31-Day Comparative Immunogenicity Study of Six DEN Vaccine Preparations and YF-Vax® Administered by a Single Subcutaneous Injection to Cynomolgus Monkeys
4. A Comparative Immunogenicity Study of Tetravalent Formulations of DEN Vaccine Preparations Administered via Subcutaneous Injection to Cynomolgus Monkeys
5. A Single-Dose Neurovirulence Study of Tetravalent ChimeriVax-DEN Vaccine Following Intracerebral Administration to Cynomolgus Monkeys
6. A Single-Dose Neurovirulence Study of ChimeriVax™-Dengue 1 Pre-Master Seed (Clone J-2-P7) and Bulk Vaccines Following Intracerebral Administration to Cynomolgus Monkeys

Monkeys inoculated by the SC routes were evaluated for viremia (Days 2-11) and immune responses (usually >Day 31) where as IC inoculated animals were monitored for clinical serology parameters as well as viremia patterns. In some experiments SC inoculated animals were challenged with WT dengue viruses to establish protections whereas the IC inoculated animals were sacrificed on Day 31 for pathology evaluations of various sections of the brains known to be involved in flavivirus pathogenesis.

### Plaque Assays and Neutralization Tests

Plaque assays for detection of chimeric viruses in sera or tissue culture supernatants were performed using Vero (for chimeric viruses) or C6/36 (for WT DEN viruses) cells as described (Monath et al., Virology 74:1742-1751, 2000) and (Guirakhoo et al., Virology 75:7290-7304, 2001). For identification of DEN serotypes in serum samples of monkeys immunized with tetravalent formulations we used an immunocytochemical focus-forming assaying in which each of four DEN serotypes are identified by immunostaining with dengue serotype-specific monoclonal antibodies (Guirakhoo et al., Virology 75:7290-7304, 2001). Neutralizing antibody titers were measured on heat-inactivated sera without addition of complement using Vero cells (Guirakhoo et al., Virology 75:7290-7304, 2001). Because chimeric viruses replicate efficiently in Vero cells and produce relatively large plaques (1-2 mm in diameter), a standard neutralization assay using double agarose overlay and neutral red was used for these viruses (Monath et al., Virology 74:1742-1751, 2000). In contrast, WT dengue viruses produce extremely small plaques (<1 mm in diameter) in Vero cells, making it difficult to count plaques after the addition of neutral red. For this reason neutralizing titers (in sera obtained after immunization with chimeras) against WT dengue viruses were measured in an immunofocus assay using DEN group reactive or serotype specific monoclonal antibodies followed by anti-mouse IgG conjugated to horseradish peroxidase (Guirakhoo et al., Virology 75:7290-7304, 2001). Fifty percent titers were determined as the last dilution of serum and virus mixture that reduced the number of plaques by 50% (compared to the virus alone).

### Statistical Analysis

Differences in responses across multiple groups and between two groups were analyzed for significance using one-way analysis of variance (Anova) and t tests, respectively (JMP software version 4.0.2).

### Results and Discussion

### Reconstruction of ChimeriVax-DEN1, -DEN3, and -DEN4 Viruses

The sequences of the prME regions of reconstructed viruses were compared to WT consensus sequences (parent DEN WT viruses from which the prME genes were derived) (Table 1). In contrast to the original chimeras, which had contained some mutations within the prME regions, all reconstructed viruses were free from unintentional mutations, with the exception of one mutation in DEN1 chimera and one nucleotide heterogeneity in the DEN3 chimera (see below). ChimeriVax-DEN1₀₀ and DEN4₀₀ viruses maintained mutations at position E494, which had been created intentionally for insertion of a NarI restriction site. In the case of ChimeriVax-DEN3₀₀ virus, it was possible to replace NarI with PvuII restriction site, which did not result in an amino acid substitution. The DEN3-specificity of the new chimeric virus was first confirmed by RT-PCR-restriction digestion analysis. The prME regions of the virus from passages 5 and 6 (P5 and P6) post transfection were then sequenced to detect mutations. The only mutation detected was a nucleotide heterogeneity (A/C) at nucleotide 535 (nucleotide 54 from beginning of the prM gene), which results in a conservative amino acid (E/D) heterogeneity at position 18 of prM. Sequencing of the corresponding plasmids revealed no mutation, indicating that this heterogeneity had occurred during in vitro virus passages. ChimeriVax-DEN1₀₀ still contained an A to G mutation, resulting in an amino acid substitution from H to R at position M39. Sequencing of the corresponding plasmids used to construct this chimera revealed the same mutation. This mutation was corrected later in a plasmid used to create the ChimeriVax-DEN1₀₁ virus, which was used to produce the cGMP vaccine virus.

### Growth Characteristics

The reconstructed ChimeriVax-DEN viruses were evaluated for their replication efficiencies in Vero cells. Their growth kinetics were compared to their predecessors, which contained mutations in the prME regions.

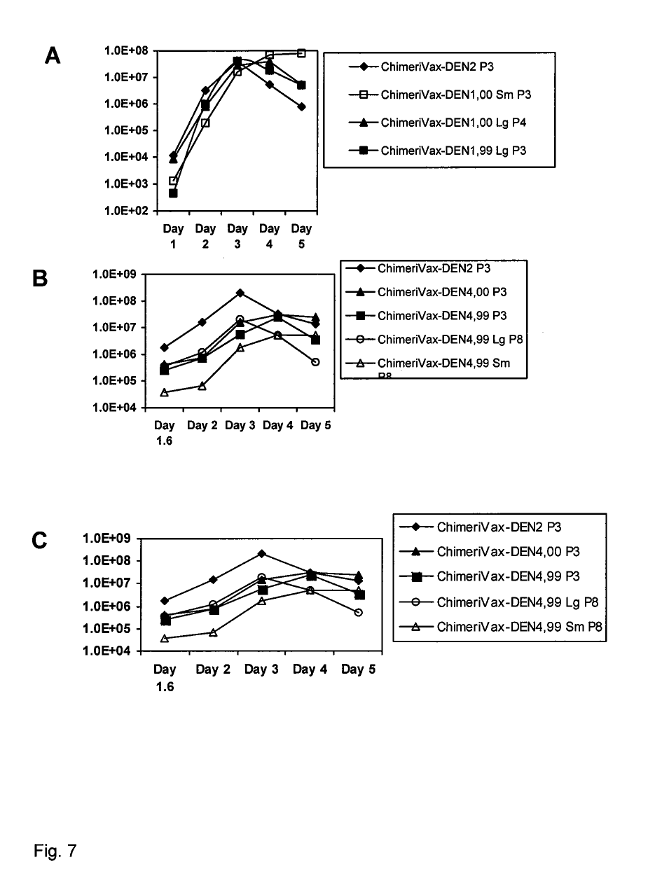

ChimeriVax-DEN1₉₉ and ChimeriVax-DEN1₀₀ viruses consisted of a mixed plaque (small and large) population. These viruses were plaque purified, and seed stocks of large (˜1.5-3.0 mm) and small (<1.5 mm) plaques were prepared for use in growth kinetic studies. A Research Master Seed (RMS) of ChimeriVax-DEN2 virus (VeroP3) was used as a control for these assays (FIG. 7A). All plaque-purified virus samples revealed similar plaque size distributions (˜1.5-3.0 mm in diameter), irrespective of their original small or large plaque sizes. Both small and large plaque ChimeriVax-DEN1₀₀ variants (tested at MOIs of 0.002 and 0.006, respectively) reached maximum titers on Day 4 (7.8 and 7.6 log₁₀ PFU/ml, respectively). In contrast, ChimeriVax-DEN1₉₉ large plaque and ChimeriVax-DEN2 RMS (tested at MOIs of 0.004 and 0.02, respectively) reached a maximum titer of 7.6 one day earlier (on Day 3).

The growth properties of ChimeriVax-DEN3₀₀ virus were examined following infection of Vero cells at an MOI of 0.01. As is shown in FIG. 7B, this virus grew to a peak titer (7.2 logs) that was one log higher than the previous construct (6.3 logs, ChimeriVax-DEN3₉₉), but somewhat lower compared to the ChimeriVax-DEN2 virus. It appears that the reversion of the intentional mutation for insertion of NarI site at E/NS1 junction (the Q to G mutation at the penultimate E492 residue) to WT sequence (PvuII virus) may have slightly improved the growth characteristics of the chimera, as compared to a Nar1 virus. The ChimeriVax-DEN3₀₀-NarI virus was reconstructed exactly as the PvuII virus (Materials and Methods), except that the Nan site was left intact. In an additional experiment to determine whether peak titers are affected by MOI, Vero cells were infected with the P5 virus at different MOIs (0.01, 0.1, and 1.0). Daily samples were collected and titrated by plaque assay. Similar curves were observed for all MOIs with titers peaking at 7.7-7.9 log₁₀ PFU/ml on Day 3 post-infection, indicating that viral titers were unaffected by 10-100 fold differences in MOI.

The growth kinetics of the reconstructed ChimeriVax-DEN4₀₀ (uncloned Vero P3) was compared to ChimeriVax-DEN4₉₉ (uncloned Vero P3) as well as to large and small plaque variants (cloned Vero P8) of ChimeriVax-DEN4₉₉ at MOI 0.01 (FIG. 7C). Both uncloned viruses peaked on Day 4 post infection (titer ˜7.5 log₁₀ PFU/ml), whereas the DEN2 control and DEN4₉₉ large plaque chimeras reached peak titers one day earlier (˜8 and 7.3 log₁₀ PFU/ml, respectively). The small plaque phenotype (see Table 1 for mutation) grew to a lower titer and peaked on Day 4 (titer ˜6.7 log₁₀ PFU/ml).

### Genomic Stability of PMS of ChimeriVax™-DEN1-4 Viruses

### I. Genetic Stability of PMS Viruses

Due to the quasi-species nature of RNA viruses, it was necessary to sequence chimeric viruses at various passage levels to assess their genetic stabilities. PMS (uncloned and cloned viruses) were sequenced and compared to the original ChimeriVax™-DEN viruses. These viruses were also passaged in vitro at MOI of ˜0.001 up to 20 passages, which is 10 passages beyond the vaccine level (P5 for uncloned and P10 for cloned viruses). Sequencing was performed directly on virus containing supernatants without additional passages in cell cultures. Sequencing data were aligned and analyzed with Sequencher 3.0 (GeneCodes) software. Nucleotide heterogeneities were registered only when a heterogeneous signal was observed in all chromatograms, representing both plus and minus strand sequencing reactions.

### I.I DEN1

The PMS candidate of DEN1 chimera (Clone J) acquired one nucleotide (nt) change from A to G, resulting in an amino acid substitution from K to R at E204. Additionally, some nucleotide heterogeneity was observed at P20. The uncloned version of this chimera exhibited a similar mutation/heterogeneity (E204) as clone J, which first appeared at P5 and then became stable through P15 (Table 3). Both uncloned and cloned viruses were free from mutations at P2 and P7, respectively. However, upon further passages in Vero (LS10) cells, both viruses acquired some mutations. The cloned DEN1 virus contained one AA substitution in the envelope and 2 nucleotide heterogeneities in NS4B gene at P20. The uncloned version contained the same envelope mutation (at E204) plus another nucleotide heterogeneity, and 2 necleotide heterogeneities within NS4B gene. The E204 mutation appeared to be advantageous for the vaccine, since it reduced both neurovirulence for infant mice (Section 0) as well as viscerotropism (measured by magnitude and duration of viremia) for monkeys in the DEN1 chimera.

### I.II DEN 2

When the PMS virus (clone A) of ChimeriVax™-DEN2 (P7) was passaged to P 10, it acquired 2 mutations: one in PrM 24 and one in E251. These mutations were stable up to P20. Clone B of this chimera underwent more mutations than clone A upon in vitro passages. Most of these mutations, however, were silent and did not result in amino acid substitutions. As expected, the uncloned version of this chimera accumulated more mutations than the cloned version (Table 4). Unlike DEN1 chimera, the mutations observed in DEN2 viruses did not affect their neurovirulence phenotype for infant mice. Clone A was selected as the PMS virus candidate and used to create cGMP MS virus (produced at Molecular Medicine, CA). The MS virus was passaged twice (to produce a research P10 vaccine level virus) in Vero LS10 cells, then tested for neurovirulence in infant mice and immunogenicity in monkeys.

I.III DEN3

Clone A of DEN3 chimera did not contain any amino acid changes up to P15. At P20, it revealed only one substitution at NS4B gene (L177F) (Table 5). Again, the uncloned version accumulated more mutations than the cloned version. Interestingly, none of the mutations, in uncloned or cloned viruses, occurred within the major envelope protein E, which contains all critical epitopes required for induction of neutralizing antibodies in a host.

### I.IV DEN 4

ChimeriVax™-DEN4 proved to be the chimera with the highest genetic stability, since it did not accumulate/tolerate any nucleotide substitutions up to P20 (Table 6).

A P10 (vaccine level) version of all 4 chimeras was produced from cGMP MS (P8). They were sequenced and tested in mice and monkeys.

### II. Genomic Stability of ChimeriVax™-DEN1-4 Viruses During GMP Manufacture

As described earlier, all four ChimeriVax™-DEN PMS (P7) were free from any amino acid substitutions in their entire genome. As expected some mutations appeared in all chimeras, except for DEN4, upon passages in Vero cells during cGMP manufactures. DEN1 acquired only one amino acid substitution (E204 K to R) as early as P8, which was stable throughout vaccine production (P10). This was an advantageous mutation, which reduced neurovirulence of this virus for infant mice as well as its viscerotropism for monkeys. At P8 (MS), DEN2 chimera showed heterogeneities (containing wild type and mutant nucleotide) in PrM and E genes, resulting in presence of mixed amino acid at PrM24 L(V) and E251 V(F). At P9, the PrM24 mutation had already been established (L to V) whereas E251 was first observed as a complete mutant population at P10. There was no amino acid substitution in DEN3 or DEN4 chimeras when passaged from P7 to P10 in Vero cells. There was one silent mutation in DEN3 (NS4a 6607) virus, which had already been observed at P7 (Table 7).

III. Genetic Stability of Chimerivax/DEN1-4 Vaccine Candidates after Passage in Vero Cells Starting with Vaccine Bulks (Aventis Pasteur, France)

Vaccine bulks of ChimeriVax/DEN1-4 viruses (passage 10, P10) were passaged to P20 in Vero-LS10 cells at an MOI of ˜0.001 pfu/cell at Aventis Pasteur to determine their in vitro genomic stabilities. All viruses remained stable during passages to P20. The sequencing data were similar to those obtained previously (see above).

### Plaque Morphology of ChimeriVax DEN1-4 GMP Vaccine Viruses

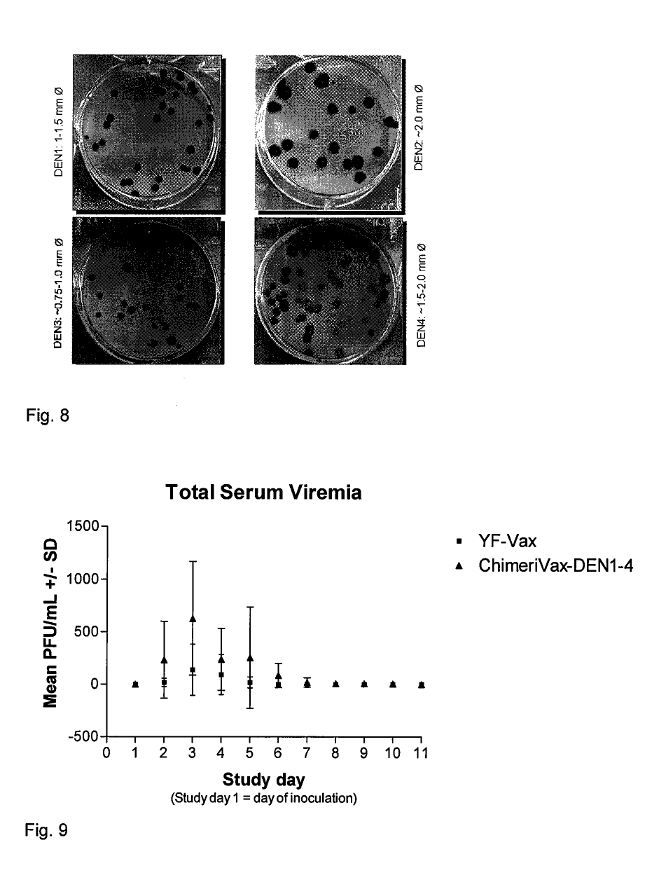

In previous studies, multiple passages of chimeras in Vero cells often resulted in adaptation, which consequently altered the plaque morphologies of these viruses. These phenotypic alterations could generally be explained with amino acid substitutions within the envelope genes of chimeras. For example, DEN1 and DEN2 PMS viruses produced small size plaques (˜1 mm) in Vero cells. The plaque sizes of these 2 chimeras increased to ˜2-3 mm upon 3 further Vero passages to produce the vaccine lots. As discussed earlier, these viruses acquired some mutations in their envelope genes. In contrast, no genomic sequence change was observed with DEN4 chimera up to P20 despite an increase in its plaque size (from ˜1 mm at P7 to ˜2.0 mm at P20). To identify the presence of a potential subpopulation of mutant viruses (with a plaque morphology different than the overall population), which would have not been detected by consensus sequencing, vaccine viruses were plagued in Vero cells. The plaque heterogeneity was determined after staining with dengue serotype specific monoclonal antibodies. As shown in FIG. 8, all viruses produced plaques of homogenous sizes except for DEN3 vaccine. The consensus sequencing of DEN3 bulk vaccine revealed no mutation despite the observation of plaque heterogeneities. However, by direct sequencing of small and large plaques from this vaccine, we were able to identify a minority population of mutant viruses (E202 K>R), which had not been identified by consensus sequencing (generally with a limit of detection of about 10%). A non-GMP version of DEN3 virus at P10 (vaccine level), which had been produced by 2 subsequent Vero passages of the cGMP MS (P8) virus, also revealed a heterogeneous plaque size population (small and large plaques). Consensus sequencing of this virus revealed nucleotide heterogeneities (containing both amino acids K and R at E202 position) within the envelope gene possibly due to a high concentration of mutant viruses (Table 9). Upon immunization of monkeys with DEN3 P10 virus, both small and large plaques, containing K and R at E202 position, respectively, could be isolated from the sera of viremic monkeys (see section on monkey experiments).

### Mouse NV of Various Passages of ChimeriVax™-DEN Viruses

I. Neurovirulence Properties of Various Clones of Den1 Chimeras

In the past, the neurovirulence test in 3-4 week old mice has been performed as a release test for ChimeriVax™-JE and -DEN2 vaccines to ensure that neurovirulence of chimeras does not exceed that of the vector (YF-VAX®) used to construct these viruses. In contrast to YF 17D, which is lethal for mice of all ages (Guirakhoo et al., Virology 257:363-372, 1999), ChimeriVax™-DEN1-4 viruses (with or without mutations) are not virulent for adult mice (3-4 weeks old). Even infant mice 8 days of age survived i.c. inoculation of DEN chimeras or their wild type dengue parent viruses (Guirakhoo et al., Virology 298:146-159, 2002). Therefore, these animals cannot be used to identify subtle changes in neurovirulence of chimeras associated with single amino acid substitutions.

A suckling mouse neurovirulence test was established that detected minor changes in the genome of chimeras involved in virulence (Monath et al., J. Virology 76:1932-1943, 2002). A suckling mouse neurovirulence protocol was adopted as a QC release test to ensure that neurovirulence of ChimeriVax™-DEN viruses is not increased when cGMP MS (P8) viruses are passaged to P10 for production of vaccine viruses. Additionally, this test was used in preclinical studies to: (a) determine neurovirulence phenotypes of different clones of DEN1 chimeras with various mutations, (b) identify possible changes in neurovirulence phenotype of chimeras when passaged from P7 to P10, and (c) assure that mutations associated with Vero passages (up to P20) do not increase the neurovirulence of dengue chimeras (see below).

In the course of development of ChimeriVax™-DEN viruses, several mutations were observed across the genome of all 4 chimeras (Guirakhoo et al., J. Virol. 75:7290-7304, 2001). These mutations were corrected in all chimeras, and the reconstructed viruses (except for DEN1 chimeras) were successfully evaluated for safety and immunogenicity in monkeys. Due to instability of DEN1 plasmids, the reconstruction of this chimera (without mutation) was not accomplished on time, and could therefore not be tested in monkeys along other reconstructed chimeras (Guirakhoo et al., Virology 298:146-159, 2002). During plaque purification in the course of PMS production for DEN1 chimera, 10 different clones (A-J) were sequenced to identify a clone without amino acid substitutions. All but 1 clone (J) contained 1 or 2 substitutions within the envelope protein E. Representative clones of DEN1 chimeras were evaluated for their neurovirulence using 4 day-old suckling mice (Table 8). All clones except clone E (this clone had 2 mutations, nucleotide 1590 A to G resulting in K to R substitution and nucleotide 3952 A to T, which was silent and did not result in any amino acid substitution) exhibited similar neurovirulence for 4 day-old mice with Average Survival Times (AST) significantly lower than with YF-VAX® (p<0.001 using log Rank Test). Clone E (E204K>R) was significantly less virulent than all other DEN1 clones (p<0.0001) with an AST of 13-15 days, significantly longer than other DEN1 clones (8.5 to 11.3 days) (Table 8). Interestingly, one of the 2 amino acid changes identified in the original DEN1 chimera was the E204 K>R substitution. This virus induced a low level of viremia (mean peak titer 0.7 log₁₀ PFU/ml) for 1.3 days when inoculated into monkeys (Guirakhoo et al., Virology 75:7290-7304, 2001). Clone J, which did not contain any mutations and was shown to be significantly less virulent than YF-VAX® in 4 days old mice, P=0.001, was selected for production of the cGMP MS virus. To determine if attenuation of clone E for infant mice correlates with a lower viscerotropism in monkeys, clone J (PMS) and clone E were compared for safety and immunogenicity in monkeys (see monkey experiments).

### II. Neurovirulence Properties of ChimeriVax™-DEN1-4 PMS Viruses Passaged in Vero Cells

### II.I Neurovirulence of P7-P10 in 4 Days Old Mice

A. Preparation of P10 Viruses

Master Seed (P8) viruses of DEN1-DEN4 chimeras were produced under cGMP at Molecular Medicine (La Jolla, Calif.) and stored at −80° C. P9 viruses (non-GMP) were produced in Vero LS10 cells infected with P8 viruses at an MOI of 0.001. The P9 viruses were tittered in a plaque assay to calculate MOI for production of P10 viruses. The titers of P9 viruses were 2.5×10⁷, 2.4×10⁷, 5×10⁶, and 5×10⁶ for DEN1-4 viruses, respectively.

To produce P10 viruses under Vero LS10 cells were washed using MEM without FBS before infection at an MOI of 0.001. Serum free growth medium was added, and flasks were incubated at 37° C., 5% CO₂ until CPE reached 10-20% (Days 3-5). When adequate CPE was observed, supernatants were harvested and clarified at 1200 rpm for 10 minutes at 4° C. Samples were then filtered through a 0.22 μM filter, and FBS to 50% concentration was added to half (to serve as positive controls, if required for stability comparisons with sorbitol) while sorbitol to 10% was added to another half (to be used in suckling mouse neurovirulence tests and monkey safety and immunogenicity studies, see below). Samples were stored at −80° C.

B. Neurovirulence of P7-P10 in Suckling Mice

Suckling mice 4 days of age were inoculated with 0.02 ml of PMS (P7), cGMP MS (P8), P9, and P10 viruses by the IC routes. Mortalities were recorded over a period of 21 days.

As shown in Table 9, neurovirulence of DEN1 chimeras for infant mice is significantly reduced from P8 to P10 passages (P<0001 for all doses measured by Log-Rank method). This reduction correlates with the appearance of E204 K to R mutations. These data are also in agreement with those shown in Table 8 where DEN1 clone E (containing a single mutation at E204R) was significantly less virulent than the parent virus with wild type sequence (E204K). For other chimeras, it appears that accumulations of mutations, such as PrM24V and E251F in DEN2, or heterogeneity in E202 residue of DEN3 at P10 do not affect their neurovirulence in mice (Table 9). Nevertheless, extensive passaging in Vero cells (P20) seems to reduce neurovirulence of these viruses for mice, in some occasions even without any apparent genetic changes (see section below). All 4 DEN chimeras were significantly less virulent (P<0001) than their vector YF 17D virus when tested at various passages (Table 9).

II.II Neurovirulence of ChimeriVax™-DEN1-4 P7 and P20 in Suckling Mice

PMS viruses were passaged in Vero cells and sequenced to assess their in vitro genetic stabilities. These mutations, which resulted in an increase in viral titers and plaque sizes, were most likely due to adaptations to Vero cells. There were some “hot spots” across the viral genome where these mutation reoccurred. For example, the E204 mutation reoccurred in DEN1, DEN2, and DEN3 viruses, and E 251 mutations reappeared in DEN1 and DEN2 chimeras (see Tables 3-5). Two hot spots were observed within the non-structural genes (e.g., NS4b177 in DEN2 and DEN3, or NS4b113 in DEN I and DEN3 viruses). To assure that the accumulated mutations during cell culture passages had not increased the neurovirulence phenotypes of these viruses, a suckling mouse neurovirulence test was performed. Four days old suckling mice were inoculated by the i.c. route with various doses of P7 (PMS) and P20 viruses. As shown in Table 10, neurovirulence of chimeras was not increased from P7 to P20. On the contrary, some chimeras lost their neurovirulence upon in vitro passages (e.g., ChimeriVax™-DEN1). Again, as observed with P10 viruses, P20 viruses were generally less neurovirulent than P7 viruses. In the case of DEN1 chimera, this was expected (due to the E204 mutation, which had previously been shown to reduce the neurovirulence of this chimera for infant mice). For other chimeras, however, reduction in neurovirulence for infant mice could not be supported by a change in the virus genome (e.g., DEN4 did not accumulate any mutations based on consensus sequencing). It is possible that subpopulations of mutants (estimated to be <10% of the total virus population) with reduced neurovirulence for infant mice are present, which can not be detected by consensus sequencing.

### III. Neurovirulence of Chimeras as Monovalent or Tetravalent Formulations

The neurovirulence phenotype of DEN chimeras at various passages as monovalent formulation has been determined previously. Some chimeras were more attenuated than others. DEN3 chimera, for example, was the least neurovirulent among all 4 serotypes. Since the vaccine will be administered as a tetravalent product it was important to address the neurovirulence phenotype of the cGMP vaccine bulks (P10) by performing a formal monkey toxicology test with the maximum concentration of these chimeras intended for human use. Prior to this test, it was necessary to demonstrate that these viruses can independently replicate in the brain without interference. Suckling mice 4 days of age were used, which had previously been shown to serve as a sensitive model for determination of neurovirulence of theses chimeras (see above).

Groups of (9-10) 4-days old infant mice (ICR) were inoculated by the i.c. route with 0.02 ml of viruses as monovalent (2.0 logs) or tetravalent formulation (2 logs of each serotype). A control group was inoculated with ˜2.0 logs of YF 17D vaccine. Actual doses were calculated by back titration of inocula in a plaque assay on Vero cells. Mice were observed for 21 days and mortalities were recorded. As expected, all 9 suckling mice in the YF 17D group died with an AST of 7.7 days. The mortality rate with monovalent chimeras was from 0 (DEN3 chimera) to 44% (DEN1 chimera, AST 13.7 days). The mortality rate in the tetravalent group was 33% (AST 14.7), which was similar to that of the monovalent chimera, with the highest neurovirulence (Table 11). This experiment demonstrated that the DEN3 chimera (non-neurovirulence for 4 days old mice) did not interfere with replication of other neurovirulent chimeras (DEN1, DEN2, and DEN4) in the brains of suckling mice when used as a tetravalent mixture. A tetravalent formulation of the vaccine bulks of DEN1-4 chimeras (5,5,5,5 logs₁₀ PFU of each serotype) was tested for neurovirulence in monkeys using a formal GLP toxicology test. In summary, the neurovirulence experiments in suckling mice revealed that:
1. Neurovirulence of ChimeriVax™-DEN1 was reduced upon Vero passage from P7 to P8.
2. Reduction in neurovirulence of ChimeriVax™-DEN1 was due to a single amino acid substitution in E protein (K to R at E204 residue).
3. The E204 mutation was stable up to the P20 passage level.
4. ChimeriVax™-DEN2 and -DEN3 viruses undergo some minor mutations when passaged in Vero cells, these mutations however do not change the neurovirulence phenotypes of these chimeras for infant mice.
5. ChimeriVax™-DEN4 did not accumulate any mutations in cell cultures (up to P20).
6. There was no interference in the replication of 4 chimeras in vivo.

### Monkey Studies

I. Safety and immunogenicity in monkeys

Four studies (Experiments 1-4) have been conducted in monkeys at Sierra Biomedical (Nevada, USA) to assess the safety and viscerotropism/immunogenicity of various ChimeriVax™-DEN viruses (prepared under non-GMP or GMP manufacture) as monovalent or tetravalent formulations (administered by the SC route). Two further studies (Experiments 5-6) were designed to address safety of ChimeriVax™-DEN viruses inoculated by the IC routes.

### Experiment 1

### Viremia and Immunogenicity of Reconstructed Chimerivax™-DEN1-4 Viruses

The objectives of this study were to determine: (1) if reconstruction of chimeric viruses to correct mutations had changed the safety (viremia) and immunogenicity profiles of the vaccine candidate; (2) if dominance of chimeric DEN2, which had been shown to have higher immunogenicity than other chimeras when used at an equal concentration (Guirakhoo et al., Virology 75:7290-7304, 2001), could be modified by reducing its dose from 5 to 3 logs; and 3) if antibodies produced in monkeys upon immunization with 1 or 2 doses of a chimeric tetravalent formulation can neutralize WT dengue viruses isolated from different geographical locations.

Because the reconstructed DEN1 (ChimeriVax™-DEN1₀₀) chimera was not available by the time these monkey experiments had started, this chimera could not be evaluated along with other reconstructed viruses. Therefore the ChimeriVax™-DEN1₉₉ was used instead.

### Safety and Immunogenicity of Reconstructed Chimeras

Twenty-two of 26 animals inoculated with chimeric DEN viruses or YF 17D (YF-VAX) control virus became viremic (exceptions included 1 animal each from Groups 1 and 3, and 2 Group 5 animals) (Table 12). The group mean peak viremia ranged from 1.8 to 2.7 log₁₀ PFU/ml. The group mean duration of viremia ranged from 3.7 to 6.3 days. The reconstructed ChimeriVax™-DEN3₀₀ and -DEN4₀₀ viruses did not significantly differ from their previous constructs (ChimeriVax-DEN3₉₉, and -DEN4₉₉ large plaque variant) in terms of magnitude and duration of viremia (p=0.2 and 0.7 for DEN4 and DEN3 chimeras, respectively, paired t tests). However, when 3 DEN4 chimeras (reconstructed, large and small plaque variants) were compared, the small plaque variant induced viremia with a lower magnitude (p=0.001, ANOVA) than other DEN4 viruses; these data were consistent with the lower in vitro growth of the small plaque virus (see Table 1 for mutation and FIG. 7 for growth curve data). The magnitude and duration of viremia for all ChimeriVax™-DEN viruses (excluding the small plaque variant of DEN4 chimera) were similar to that of the YF-17D control virus (p=0.17 and 0.35 for magnitude and duration of viremia, respectively, ANOVA).

All monkeys seroconverted after one dose of monovalent vaccines, as measured by a rise in neutralizing antibody titer to homologous virus on Day 31. The Geometric Mean Titers (GMT) ranged from 640 to 2560 (Table 13). When responses were compared across groups, including the YF 17D control, the large plaque variant of ChimeriVax-DEN4 appeared to be the most immunogenic virus (P=0.027, ANOVA), followed by YF 17D. However, when neutralizing antibodies were compared between reconstructed viruses and their predecessors, the differences were not statistically significant (p=0.16 and 0.075 for chimeric DEN3 and DEN4 viruses, respectively, t tests).

### Dose Adjustment of DEN2 Chimera in the Tetravalent Formulation

All animals inoculated with the tetravalent formulation (containing a nominal dose of 5 logs of each DENT, DEN3, and DEN4 and 3 logs of DEN2 virus) became viremic (Table 14). The group mean peak viremia in monkeys immunized with tetravalent chimeras (2.8 log₁₀ PFU/mL) was significantly lower than that of the WT tetravalent formulation (4.3 log₁₀ PFU/mL) (p=0.002, t test). The group mean duration of viremia was also lower in these monkeys, but the difference was not statistically significant (p=0.13, t test).

To determine if the dose adjustment for chimeric DEN2 (nominal 3 logs) had resulted in a lower level of viremia and/or a delay of the onset of viremia, we tested daily sera (obtained from Day 1 to Day 12 post immunization) for the presence of each DEN serotype by an immunofocus assay. The spectrum of serotypes present in monkey sera immunized with chimeric tetravalent viruses differed with inocula, whereas 3 of the 4 monkeys immunized with WT parent viruses were viremic with all 4 DEN serotypes (DEN4 virus was not detected in one monkey (14280F) (Table 15A). ChimeriVax-DEN1, -DEN2, and -DEN3 viruses were detected in 2/6, 1/6, and 0/6 monkeys of Group 9, respectively. The most frequently detected chimeric virus was ChimeriVax-DEN4, which was present for several days in all 6 monkeys (Table 15B). The magnitude and duration of viremia in the ChimeriVax tetravalent group were significantly lower than in the WT tetravalent group (p=0.0001, ANOVA). Among WT viruses, DEN1 and DEN2 induced a higher magnitude and longer duration of viremia than DEN 3 and DEN4 viruses (p=0.008, ANOVA for both magnitude and duration of viremia). However, among chimeric viruses, ChimeriVax-DEN4₀₀ induced a significantly higher magnitude (p=0.0004, ANOVA) and duration (p=0.0001, ANOVA) of viremia than the other 3 chimeras. The onset of ChimeriVax-DEN2 viremia appeared to be delayed compared to other chimeras, since no virus could be detected in monkey R14207M (the only monkey positive for DEN2 chimera) until Day 7 (Table 15B).

All monkeys immunized with 1 dose of the tetravalent formulation (WT or chimeric dengue) developed neutralizing antibodies against all 4 serotypes. The GMT ranged from 254 (Group 9 sera against chimeric DEN1) to 2153 (Group 8 against WT DEN2) (Table 16). The neutralizing antibody titers were significantly higher in Group 8 than Group 9 (p=0.025, ANOVA). The highest response in Group 8 animals was directed against DEN2 virus, however, this was not significant (P=0.57, ANOVA). In contrast, the highest response in Group 9 animals was directed against DEN4 chimera, which was highly significant (P=0.009, ANOVA).

### Neutralizing Antibody Responses to Homologous (ChimeriVax-Den) and Heterologous (WT DEN Isolates) Viruses After 1 or 2 Doses

Animals in Group 9 received a 2^{nd} dose (the same formulation as the first dose) 2 months after the first dose. No viremia was detected after the second dose (level of detection was 5 PFU/ml of serum). The neutralizing antibody titers were measured against homologous (chimeric DEN) or heterologous (WT DEN (see Materials and Methods)) viruses using sera obtained pre-(Day 1) and post-(Day 31) booster immunizations. All 6 animals developed neutralizing titers against all serotypes tested after a single dose. The response was generally higher against homologous (chimeric DEN) than heterologous viruses. The highest response was directed against ChimeriVax™-DEN4 virus (GMT=3225). Responses against WT DEN isolates varied from 80 (GMTs against DEN1 from Jakarta and DEN3 1325 from Sri Lanka) to 1613 (GMTs against DEN4 P75-215 from Malaysia). After the booster dose, all titers increased (P=0.053, t test, when titers against all viruses were compared before and after the booster dose). However, when GMTs (before and after booster immunization) were compared for individual virus isolates, only increases against DEN2 PR 159 from Puerto Rico (P=0.05, t test), ChimeriVax™-DEN4 (P=0.01, t test) and DEN4 1228 from Indonesia (P=0.057, t test) were statistically significant (Table 17).

### Experiment 2

### A 31-Day Comparative Immunogenicity Study of Three YF/DEN-1 Vaccines Administered by a Single Subcutaneous Injection to Rhesus Monkeys

ChimeriVax™-DEN1 PMS virus acquired one mutation (resulting in an amino acid change from K to R at position E204) when passaged under laboratory conditions or cGMP manufacture to produce the working seed (see section on genetic stability above). This mutation, which was stable throughout manufacturing as well as multiple Vero passages (up to P20), increased the plaque size but attenuated the virus for 4-days old mice when inoculated by the i.c. route. The effect of this mutation on viscerotropism (induction of viremia) of the virus was assessed by inoculation of monkeys with ChimeriVax-DEN1 viruses with (clone E, P6) or without (clone J, P7) the E204 mutation. The original DEN1 chimera (ChimeriVax-DEN-1, uncloned P4, 1999) was selected as a control, because its viremia and immunogenicity profiles had already been evaluated in monkeys as a monovalent or a tetravalent (combined with 3 other chimeras) vaccine (Guirakhoo et al., Virology 75:7290-7304, 2001).

Groups of 4 rhesus monkeys were inoculated with 5 log₁₀ PFU/0.5 ml of DEN1 chimeras. Viremia was measured (by plaque assay on Vero cells) in sera obtained from days 2 to 11 post infection.

All monkeys inoculated with DEN1 PMS virus (clone J, Group 3) became viremic, whereas 3/4 and 2/4 monkeys inoculated with clone E or uncloned viruses, respectively, became viremic. The mean peak virus titer (2.5 log₁₀ PFU/ml) and duration (8.5 days) of viremia in Group 3 monkeys was significantly higher (p=0.024 and 0.0002 for peak virus titer and duration, respectively) than in Groups 1 and 2). Despite the lack of viremia in some monkeys, all animals developed neutralizing antibody titers against homologous viruses. Consistent with the level of viremia, the neutralizing titers in monkeys immunized with the PMS virus (without mutation) were higher than in the other 2 groups (p=0.0002). The sera of Group 1 monkeys (immunized with a DEN1 chimera with 2 amino acid substitutions on the envelope proteins, M39 H>R and E204 K>R) revealed the lowest neutralizing titers, indicating that the M39 mutation may have further attenuated the virus (p=0.0045). These experiments demonstrated the possibility of a direct correlation for ChimeriVax™-DEN1 viruses between: (1) the magnitude of viremia and the level of neutralizing antibodies in monkeys, and (2) neurovirulence of chimera for mouse and viremia/immunogenicity in monkeys (clone E was attenuated for 4 days old mice and induced a lower level of viremia and neutralizing antibodies than the PMS virus, which was neurovirulent for mice of similar age).

In summary, the mutation from K to R at E204 residue of ChimeriVax™-DEN1 reduced the replication of the DEN1 chimera in vertebrate hosts, as shown by low levels of viremia and neutralizing responses. Mutation of this residue, which is conserved in all dengue serotypes, can thus be used in the construction of chimeras with desired phenotypes appropriate for human dengue vaccine.

### Experiment 3

### A 31-Day Comparative Immunogenicity Study of Six DEN Vaccine Preparations and YF-Vax® Administered by a Single Subcutaneous Injection to Cynomolgus Monkeys

Safety and immunogenicity of original as well as reconstructed chimeras as monovalent or tetravalent formulations were evaluated in rhesus monkeys and reported previously (Guirakhoo et al., Virology 257:363-372, 1999; Guirakhoo et al., J. Virol. 74:5477-5485, 2000; Guirakhoo et al., Virology 298:146-159, 2002). The current DEN1-4 PMS viruses (P7) had acquired one or two mutations when passaged under laboratory conditions (P7 to P10) or under cGMP manufacture to produce cGMP MS virus stocks (P8). Some of these mutations (see section genetic stability) were different than those reported for reconstructed chimeras (Guirakhoo et al., Virology 298:146-159, 2002). Moreover, previously constructed chimeras had been evaluated in rhesus species, which currently are difficult to obtain for preclinical studies of human vaccines. The current experiment was conducted in cynomolgus monkeys as a possible replacement for rhesus monkeys and was performed as a pilot (bridging) experiment for the subsequent tetravalent study (see Experiment 4) to ensure that safety and immunogenicity of cGMP vaccine viruses (P10) could be evaluated in these animals.

The main objectives of this study were:
To characterize viral replication (duration and magnitude of viremia) and immune response (neutralizing antibody production) resulting from a single subcutaneous administration of four investigational DEN vaccine preparations, administered as monovalent or tetravalent preparations,
To assess the feasibility of this approach for safety evaluations of additional laboratory-passaged dengue vaccine stocks by comparing results from the investigational DEN2 (passage 10) vaccine preparation with those obtained using clinical-grade DEN2 cGMP vaccine (passage 9); and
To evaluate the immunogenicity of the control vaccine YF-Vax® in cynomolgus monkeys.

Twenty-one cynomolgus monkeys were divided into 7 groups. Animals received one dose of ChimeriVax™-DEN1-DEN4 monovalent viruses (Groups 1-4) prepared at P10 from cGMP MS stock viruses (P8) produced at Molecular Medicine (La Jolla, Calif.). Controls included the ChimeriVax™-DEN2 vaccine, which was successfully tested as a monovalent vaccine in human volunteers (Group 5), and YF-VAX® (Group 7). A tetravalent mixture of P10 viruses was applied to assess safety and immunogenicity of these viruses when mixed at equal concentrations of 5.0 logs each (Table 20)

All vaccine preparations were well tolerated in male and female cynomolgus monkeys throughout a 30-day postdose observation period. There were no vaccine-related changes in clinical signs or body weight. Transient, low magnitude viremia was noted for all treatment groups (Table 21). The severity of viremia for all vaccines remained within acceptable limits, according to the WHO guidelines established for yellow fever vaccines (i.e., <500 mouse IC₅₀ units for any individual monkey, estimated to equal 4.3 log₁₀ (19,952) Vero cell PFU/ml for YF-Vax®).

All three YF-Vax® control vaccine- and DEN2 cGMP reference vaccine-treated monkeys (Groups 7 and 5, respectively) developed viremia with peak viral titers ranging from 1.0 to 2.0 log₁₀ PFU/ml on Days 3-5 and 7-10, respectively. Peak titers were slightly lower than those observed in prior studies using rhesus monkeys (for YF-Vax®, see Guirakhoo et al., Virology 298:146-159, 2002; for DEN2 cGMP reference vaccine, see BB-IND#10211, Section SBi Study No. 1128-88), and the onset of viremia was slightly delayed relative to the time course observed with rhesus monkeys.

Anti-flavivirus antibodies were present in sera of all monkeys at 30 days post-vaccination and generally showed greater neutralizing activity to homologous vaccine virus strains than to heterologous wild-type virus strains (Tables 22 and 23). The level of neutralizing antibodies in ChimeriVax™-DEN P10 viruses was higher than in control groups (ChimeriVax™-DEN2 GMP vaccine and YF-VAX®). Tetravalent inoculation containing 5.0 logs of each monovalent component produced high levels of serotype specific neutralizing antibodies in all monkeys.

In summary, it appears that P10 viruses are safe and highly immunogenic in cynomolgus monkeys, and that these animals can replace rhesus monkeys for evaluation of monovalent or tetravalent ChimeriVax™-DENT-4 viruses.

Previously, in vitro passaged as well as cGMP manufactured ChimeriVax™-DEN1-4 viruses were sequenced, and their genetic stabilities and neurovirulence phenotypes in suckling mice were assessed. ChimeriVax™-DEN1 acquired one mutation in E protein that attenuated virus for suckling mice 4 days of age. The mutation found in ChimeriVax-DEN3 and DEN2 viruses during Vero passages seemed to have less effect on neurovirulence phenotypes of these viruses when assessed in suckling mice inoculated by the i.c. route. DEN4 chimera did not undergo any mutations when passaged under manufacturing or research setting up to P20.

To determine the genetic stabilities of theses viruses in vivo (in host), we isolated plaques of various sizes from sera of monkeys inoculated with monovalent chimeras and sequenced the envelope gene regions. The strategy was to isolate plaques from the last day of viremia to allow for maximum mutations/selection in the host.

Three plaques were isolated from monkey F20515M inoculated with ChimeriVax™-DEN1: One small plaque (˜1 mm) isolated from Day 10 (viremia titer 1.8 log₁₀ PFU/ml) was amplified (once) in Vero PM cells and sequenced. An nucleotide change from G to T at position 1591 resulted in an amino acid change at E204 from R to N. Two large plaques (˜5 mm) were isolated from Day 11 (viremia titer 2.7 log₁₀ PFU/ml), amplified (once) in Vero PM cells and sequenced across prME. Both viruses contained mutations at nucleotide position 2030 from G to T, resulting in an amino acid change at E351 from V to L. The L amino acid at this residue is common in many flaviviruses such as dengue 2, dengue 3, Tick-borne encephalitis, Japanese encephalitis, and yellow fever.

Two large plaques (˜8 mm) were isolated from the last 2 days of viremia (Days 10 and 11, titer 2.0 and 1.0 log₁₀ PFU/ml, respectively) from the only viremic monkey in the ChimeriVax™-DEN2 group (monkey F20514M). All plaques detected in this monkey were homogenous and of large size (˜8 mm). The 2-isolated plaques were amplified in VeroPM cells (once) and sequenced. The sequences were identical to the inoculated virus (P10) which contained a nucleotide change 1730 G>T resulting in an amino acid change at E251V>F.

For ChimeriVax™-DEN3 group, 2 plaques (one small ˜1 mm, and one large ˜2 mm) were isolated, amplified in Vero cells and sequenced across PrME region. The small plaque had no mutations, whereas the large plaque showed two: nucleotide1402 T>C (silent) and nucleotide 1584 A>G (E202 K>R). A nucleotide heterogeneity at E202 K/R had been detected when ChimeriVax™-DEN3 was produced in Vero cells (Table 9).

Homogeneous plaque sizes (˜2 mm) were noticed in sera of all three monkeys inoculated with ChimeriVax™-DEN4 virus. One plaque was isolated from the last viremia day from each of three monkeys [(F20513M, 2 mm, Day 6, 1.7 log₁₀ PFU/ml), (F18131M, 2 mm, Day 5, 2.2 log₁₀ PFU/ml), and (F205109F, 2 mm, Day 7, 1.0 log₁₀ PFU/ml)]. The sequencing of all three viruses revealed no mutations (identical to inoculated P10 virus).

Since amplified monkey virus isolates from ChimeriVax™-DEN1 and DEN3 contained some mutations, it was necessary to determine if their neurovirulence phenotypes are altered. Groups of 4 day old suckling mice were inoculated by the i.c. route with various dilutions of both viruses. Inoculated doses were determined by back titration of inocula in a plaque assay using Vero cells. DEN2 and DEN4 plaque isolates were not tested because their sequences were identical to the inoculated viruses already tested in suckling mice. DEN1 small plaque virus killed 5/11 mice with AST of 12.2 days at a dose of 2.2 logs PFU. At a ten-fold dilution, this virus killed only 3/11 mice with an AST of 11.3. In contrast, the large plaque isolate of YF/DEN1 was significantly more virulent than the small plaque (P=0.006 according to the log-Rank method) and killed 10/11 mice (AST 10.9). Both viruses were, however, less virulent than YF-VAX® when administered at ˜0.5 logs higher dose (P<0.0001 according to the log-Rank method) (Table 24).

In summary, it appears that, similar to in vitro tests, DEN1 and DEN3 undergo some mutations in vivo (in monkeys). As expected, DEN3 mutations are inconsequential in terms of neurovirulence for suckling mice. Mutations in the DEN1 chimera, however, reverted neurovirulence of these chimeras for suckling mice similar to that of their PMS virus. Despite a slight increase in neurovirulence, all viruses isolated from monkeys were significantly less neurovirulent than the YF 17D vaccine. To assure the safety of DEN1 for humans (in case of a revertant to wild type sequence at position E204), we performed a monkey toxicology test in which both wild-type and mutant DEN1 chimeras were inoculated into monkeys by the i.c. route (see Experiment 6).

In sum, cynomolgus monkeys tolerated single subcutaneous injections of ˜1.0×10⁵ PFU (i.e., 4.0×10⁵ PFU total inoculum for the tetravalent vaccine) of experimental DEN vaccine preparations, clinical grade DEN2 cGMP vaccine, or YF-Vax® vaccine throughout a 30-day postdose observation period without clinical signs or effects on food consumption or body weight.

Transient viremia with vaccine-specific profiles were noted with all preparations and these remained within acceptable limits, according to the WHO guidelines established for yellow fever vaccines (i.e., <500 mouse IC50 units for any individual monkey, estimated to equal 4.3 log₁₀ (˜19952) Vero cell PFU/ml for YF-Vax®).

Virus serum titers were detected in only 1 of 3 monkeys injected with DEN1 or DEN2 vaccine preparation, but in all monkeys injected with DEN3 or DEN4 vaccine preparation. Generally, higher serum virus titers were associated with earlier onset of viremia following injection. Viremia was detected in all monkeys injected with tetravalent DEN vaccine (i.e., a mixture of DEN1, DEN2, DEN3, and DEN4), but the profile of viral strains detected, the day of viremia onset, and the duration of viremia showed considerable inter-monkey variability with DEN1 titers absent from all monkeys and DEN4 titers present in all monkeys.

Anti-dengue antibodies were present in sera of all monkeys at 30 days post-injection, and generally showed greater neutralizing activity to homologous vaccine-strain virus than to heterologous wild-type virus.

A comparison of neutralizing antibody titers showed type-specific induction patterns and reactivity to vaccine-strain and wild-type virus following injection of either monovalent or tetravalent vaccine preparations. Investigational DEN2 (passage 10) vaccine preparation induced substantially better neutralizing antibody titers than clinical-grade DEN2 cGMP vaccine (passage 9). It is possible that mutations occurred in DEN2 P10 (PrM24 L to V and E251 V to F are responsible for increase in immunogenicity of this virus vs. DEN2 cGMP (P9) with only some heterogeneity in PrM 12 M/I (Table 8.2 Section 8.0 BB-IND#10211)

All monkeys injected with YF-Vax® vaccine or DEN2 cGMP vaccine developed viremia with peak viral titers ranging from 1.0 to 2.0 log₁₀ PFU/ml on Days 3-5 and 7-10, respectively. Peak titers were slightly lower than those observed in prior studies using rhesus monkeys (YF-Vax®, and YF-DEN2 cGMP reference vaccine; Guirakhoo et al., J. Virol. 74:5477-5485, 2000), and the onset of viremia was slightly delayed relative to the time course observed with rhesus monkeys. Vaccination with YF-Vax® induced approximately four-fold lower neutralizing antibody titers than were previously observed with rhesus monkeys (Guirakhoo et al., Virology 298:146-159, 2002). The apparent disparity in immunogenicity between the studies is likely related to slightly different interstudy viremia profiles.

Some mutations were observed in DEN1 and DEN3 chimeras isolated from monkeys, some of these mutations had been already observed during Vero culture passages and been extensively characterized for their impact on safety and immunogenicity in mice and monkeys (e.g., DEN1 204 mutation). DEN2 and DEN4 chimera did not go under genetic changes when inoculated into monkeys and isolated on the last day of viremia.

### Experiment 4

### A Comparative Immunogenicity Study of 4 Tetravalent Formulations of DEN Vaccine Preparations Administered Via Subcutaneous Injection to Cynomolgus Monkeys

The main objectives of this study were:
to characterize the viral replication (the duration and magnitude of viremia);
to determine immune response (neutralizing antibody production) in cynomolgus monkeys following subcutaneous administration of 4 tetravalent DEN cGMP vaccine preparations that differ in their respective concentrations of each constitutive DEN strain; and
to demonstrate protection against wild-type dengue serotypes 1-4 challenge strains.

Four tetravalent vaccine suspensions that were mixtures of 4 chimeric yellow fever virus (strain 17D)/dengue virus vaccine strains (P 10) were prepared in Vero (LS10) cells under cGMP in Aventis Pasteur (Marcy l'Etoile, France) and stabilized in 10% sorbitol. Twenty-four cynomolgus monkeys were given a single subcutaneous injection with of one 4 tetravalent formulations Groupl (5,5,5,5 log₁₀PFU); Group 2 (3,5,5,3 log₁₀ PFU); Group 3 (5,5,5,3 log₁₀PFU) and Group 4 (3,3,3,3 log₁₀ PFU). The monkeys were evaluated for changes in clinical signs and body weight. Blood samples were collected and assayed for viremia and antibody titer at various time points.

Viremia was measured on sera obtained from Days 2-11. First, total viremia was measured to identify if these formulations remained within acceptable limits according to the WHO guidelines established for yellow fever vaccines (i.e., <500 mouse IC₅₀ units for any individual monkey, estimated to equal 4.3 log₁₀ Vero cell PFU/ml for YF-Vax®) (WHO98). Next, the presence of each of four ChimeriVax™-DEN viruses in sera of monkeys from Days 2-11 was identified using a serotype-specific immuno focus assay on Vero cells (Guirakhoo et al., J. Virology 75(16):7290-7304, 2001).

To assess protective immunity conferred by administration of one dose of 4 different tetravalent formulations, all monkeys were challenged with WT dengue challenge strains provided by Dr. Kenneth Eckels (WRAIR, USA) 6 months post immunization. Monkeys were randomized into 4 new Groups. A Control group consisted of 16 animals (4 monkeys per serotypes) that had been shown to be flavivirus naïve by prescreening for presence of JE and DEN1-4 neutralizing antibodies by PRNT. Since the suitability of the challenge viruses has not yet been established in cynomolgus monkeys, 8 of 16 control monkeys (2 per serotype) were inoculated in advance with WT challenge strains to assure that these viruses will induce acceptable levels of viremia in these monkeys. Viremia and neutralizing antibodies were measured post challenge to determine protection against each dengue serotype.

The WT challenge strains of DEN which were used in the challenge are: DEN1 D1 (west pacific74) L/N1,84′); DEN2 D2 (S16803 PDK-10) L/N1-90); DEN3 D3 (CH53489) L/N 450); DEN4 D4 strain 341750 (Carib).

### Total Viremia

Total viremia was measured from sera obtained on Days 2-11 in a plaque assay on Vero cells and is shown in Table 25. All monkeys became viremic. Onset of viremia in high dose formulation (5,5,5,5, Group 1) was as early as Day 2, and all animals were viremic on Day 3. No viremia was detected in any monkey after Day 8 (level of detection ≦1.7 log₁₀ PFU/ml). The mean peak and duration of viremia for this group were 2.6 log₁₀ PFU/ml and 4.0 days, respectively (Table 26). In low dose Group animals (3,3,3,3, Group 4), viremia was delayed until Day 3 post immunization. Only one animal showed border line viremia of 1.7 log₁₀ PFU/ml on this day. All animals were viremic on Day 6 until the last day of serum collection (Day 11). The mean peak and duration of viremia were 2.5 log₁₀ PFU/ml and 5.5 days, respectively (Table 26). Viremia in Groups 2 and 3 (where doses of DEN1 and/or DEN4 chimeras had been reduced) were observed from Day 2 until Day 11, indicating a possible circulation of different serotype(s) on each specific date. In contrast to Groups 1 and 4, where all animals were viremic in a given day, no single day could be identified in which all animals in Groups 3 or 4 were viremic. The lowest mean peak viremia (2.1 log₁₀ PFU/ml) was identified in Group 3 where animals had received a reduced dose of ChimeriVax™-DEN4 virus (Table 26). The shortest duration of viremia (2.5 days) was observed in Group 2, where animals had received reduced doses of DEN1 and DEN4 chimeras (Table 26).

### Serotype Specific Viremia

ChimeriVax™-DEN1-4 viruses were detected in serum samples obtained from Days 2-11 post immunization using IFF assay on Vero cells as described previously (Guirakhoo et al., J. Virology 75(16):7290-7304, 2001). As shown in Table 27, ChimeriVax™-DEN1-4 viruses could be detected in all 4 groups inoculated with different formulations (level of detection 1.3 log₁₀ PFU/ml). The following serotypes were detected in sera of monkeys within Groups 1-4:

Group 1 (5,5,5,5): All 4 serotypes were detected in 4/6 monkeys on Day 3. Up to Day 7, DEN3 and DEN4 could be isolated from some monkeys, but DEN2 and DEN1 chimeras could not be detected beyond Days 4 and 5, respectively. DEN1, DEN2, and DEN2 chimeras could not be found in monkeys F20967 and F21339M, respectively.

Group 2 (3,5,5,3): In this group, where monkeys were immunized with 100-fold lower doses of ChimeriVax™-DEN1 and -DEN4, no DEN1 chimera could be detected in any monkeys. Other chimeras were detected from Day 2 until Day 11.

Group 3 (5,5,5,3): Most of monkeys showed early viremia with DEN1-3 and a delay in viremia (replication) of DEN4 chimera. DEN1, 2, 4 and DEN1, 3, 4, could not be detected in monkeys F21544M and F21149, respectively.

Group 4 (3,3,3,3): No chimera could be detected in any monkeys as early as Day 2, with the exception of one animal (monkey F21522M, in which DEN2 chimera could be detected on Day 2). In contrast to Group 1 (high dose) most viruses were detected around Day 3-4 and no virus was detected beyond Day 7, majority of viruses were detected between Days 5-7. DEN3 and 4 chimeras could be detected until the last day of serum collection (Day 11). DEN1 could not be found in monkeys F21522M and F21570F (Table 27).

### Magnitude and Duration of Serotype Specific Viremia

The magnitude and duration of viremia induced by each serotype in individual monkeys are described below:

Group 1 (5,5,5,5): All monkeys became viremic to DEN3 and DEN4 chimeras. In 5/6 and 4/6 animals DEN1 and DEN2 could be detected, respectively. The highest peak viremia, which lasted for 6 days, was induced by DEN4 chimera (monkey F21786F). The mean peak viremia titers were 1.5, 1.6, 2.3, and 1.9 for DEN1-4 viruses, respectively. The mean duration of viremia was 1.8, 1.2, 2.3, and 3.7 days for DEN1-4 viruses, respectively (Table 28).

Group 2 (3,5,5,3): None of the monkeys became viremic to DEN1 (reduced dose of 3 logs). Four/6 monkeys became viremic to DEN2 and DEN3 viruses (maximum dose of 5 logs). Only 2/6 monkeys became viremic to DEN4 component (reduced dose of 3 logs). The highest viremia level (3.4 log₁₀ PFU/ml), which last for 3 days, was found with DEN3 in monkey F21565F. The highest duration of viremia was 8 days with DEN4 virus in monkey F21355M. The mean peak viremia titers were 0, 2.3, 2.6, and 2.7 for DEN1-4 viruses, respectively. The mean durations of viremia were 0, 2, 4.5, and 4.5 days for DEN1-4 viruses, respectively (Table 28).

Group 3 (5,5,5,3): All monkeys became viremic to DEN3. In 5/6 and 4/6 animals DEN2 and DEN1, DEN4 viruses could be detected, respectively. The highest peak viremia (3.1 log₁₀ PFU/ml), which lasted for 3 and 7 days, respectively, was induced by DEN3 and DEN4 chimeras (monkeys F21342M and F21384F). The mean peak viremia titers were 1.8, 1.7, 2.2, and 2.1 for DEN1-4 viruses, respectively. The mean durations of viremia were 2.2, 2.4, 2.6, and 2.8 days for DEN1-4 viruses, respectively (Table 28).

Group 4 (3,3,3,3): All monkeys became viremic to DEN2 and DEN4 viruses. DEN3 and DEN1 viruses could be detected in 5/6 and 4/6 animals, respectively. The highest peak viremia (3.1 log₁₀ PFU/ml) was induced by DEN4 chimera (Monkey F209108F), and lasted for 4 days. The longest duration of viremia (8 days) was detected with DEN4 chimera (monkey F2131M) with a peak magnitude of 2.8 log₁₀ PFU/ml. The mean peak viremia titers were 1.9, 1.9, 2, and 2.5 for DEN1-4 viruses, respectively. The mean duration of viremia was 3, 3.8, 4.2, and 5.3 days for DEN1-4 viruses, respectively (Table 28).

### Neutralizing Antibody Response

The neutralizing antibody responses were measured on Days 1 (pre-immune), 31 and 120. A Plaque Reduction Neutralization Test (PRNT) was performed on Vero cells using homologous ChimeriVax™-DEN1-4 viruses and heat inactivated monkey sera without addition of complement, as described previously (Guirakhoo et al., J. Virology 75(16):7290-7304, 2001).

Sera obtained on Day 1 (pre immunization) were negative (PRNT₅₀<10), with the exception of sera from monkeys F20967F and F21343M (PRNT₅₀ of 10, Group 5,5,5,5), F21565 and F21501M (PRNT₅₀ of 10, Group 3,5,5,3) and monkey F21544M (PRNT₅₀ of 10, Group 5,5,5,3). All monkeys (6/6) in the high dose Group 1 (5,5,5,5) were seroconverted to all 4 ChimeriVax™-DEN1-4 viruses by Day 31 post immunization and remained seropositive (PRNT₅₀≧10) on Day 121 (Table 28). The level of dengue specific neutralizing antibodies varied from 160 to 2560 for DEN1, DEN3, and DEN4 viruses, and 40 to 5120 for DEN2 virus (Table 28). The GMTs on Day 31 were 452, 508, 452, and 508 for DEN1-4 viruses, respectively. The GMTs on Day 121 were 359, 359, 1016, and 508 for DEN1-4 viruses, respectively.

All monkeys in the low dose Group 4 (3,3,3,3) were also seroconverted to all 4 ChimeriVax™-DEN1-4 viruses by Day 31 post immunization. However, on Day 121 only 5/6 remained seropositive to all 4 DEN viruses; Monkey F21522M became seronegative to DEN4 when tested on Day 121 (the PRNT₅₀ dropped from 640 on Day 31 to <10 on Day 121, Table 29). The level of dengue specific neutralizing antibodies varied from 20 to 1280 for DEN1 and DEN3 viruses, 40-2560 for DEN2 virus, and 10-1280 for DEN4 virus (Table 29). The GMTs on Day 31 were 254, 359, 285, and 452 for DEN1-4 viruses, respectively. The GMTs on Day 121 dropped to 63, 320, 254, and 34 for DEN1-4 viruses, respectively.

Only 3/6 monkeys in the dose-adjusted formulation, Group 3 (3,5,5,3) were seroconverted to all 4 ChimeriVax™-DEN1-4 viruses by Day 31 post immunization. Three monkeys (F212117F, F20977F, and F21534M) were seroconverted to DEN1-3 viruses but not to DEN4 virus. Interestingly, monkey F212117F did seroconvert to DEN4 on Day 121 (PRNT₅₀ of 640), whereas the other 2 monkeys remained seronegative to DEN4, increasing the number of seroconverted animals to 4/6 on Day 121. It is possible that the replication (therefore induction of neutralizing antibodies) of DEN4 chimera in this monkey was delayed due to unknown genetic factors of the host and/or low dose of this chimera (3 logs). As shown in Table 27, DEN4 chimera was the only virus which was detected in this monkey on the last day of viremia (Day 11) and no DEN4 chimera could be detected in sera of monkeys (F20977F, and F21534M), which did not seroconvert to DEN4 chimera on Days 31 and 121 (Table 27). The level of dengue specific neutralizing antibodies varied from 20 to 1280 for DEN1, 20-5120 for DEN2, 10-1280 for DEN3, and 10-640 for DEN4 viruses. The GMTs on Day 31 were 90, 285, 254, and 10 for DEN1-4 viruses, respectively. The GMTs on Day 121 were slightly higher than Day 31 and were 101, 452, 160, and 29 for DEN1-4 viruses, respectively.

Only 4/6 monkeys in Group 4 (5,5,5,3) were seroconverted to all 4 ChimeriVax™-DEN1-4 viruses by Day 31 post immunization, whereas all monkeys (6/6) became seropositive to all 4 serotypes on Day 121. Two monkeys (F21544M and F21149M), which were seronegative to DEN4 on Day 31, developed high titers (PRNT50 320, and 2560 respectively) of neutralizing antibodies to DEN4 measured on Day 121 post immunization. Similar to the situation mentioned in above, monkey F21544M was viremic to only DEN4 on the last day of viremia sample (Day 11). Moreover, no DEN4 virus could be detected (up to Day 11) in monkey F21149M, which may indicate a delay (>Day 11) in replication of this virus due to its reduced dose (3 logs of DEN4) in tetravalent 5,5,5,3 formulation (Table 27).

The GMTs on Day 31 were 226, 452, 275, and 26 for DEN1-4 viruses, respectively. The GMTs on Day 121 (slightly higher for DEN4 chimera) were 142, 508, 320, and 718 for DEN1-4 viruses respectively.

Viremia was dose dependent, which was early in the high dose group (Day 2 to 7) and late in the low dose group (Day 3 to 11). When concentration of one or 2 components in a tetravalent formulation was reduced, the corresponding viruses were either non detectable (e.g., DEN1 in 3,5,5,3 formulation) or detected very late (e.g., DEN4 in 3,5,5,3 and 5,5,5,3 formulations). Generally, DEN3 and DEN4 chimeras induced viremia with a higher magnitude and duration than those of DEN1 and DEN2 chimeras when applied at equivalent concentrations.

All monkeys in both high and low dose groups were seroconverted to all 4 serotypes on Day 31 post-immunization. All monkeys remain seropositive to all 4 serotypes on Day 121, with the exception of one monkey in the low dose group, which became seronegative to DEN4 virus. Upon reduction of DEN4 virus concentration in 5,5,5,3 formulation, only 4/6 monkeys were seroconverted to all 4 serotypes (2 monkeys did not seroconvert to DEN4) on Day 31. However on Day 121 all monkeys became seropositive to all 4 dengue serotypes, again indicating a delay in replication and induction of immune response against DEN4 component of the tetravalent formulation.

The GMTs in the high dose group animals, measured on Day 31 and Day 121 post immunization, were higher than those in the low dose group (Table 30).

All animals, with the exception of F20977F and F21534 which did not seroconvert to DEN4 virus, were randomized into 4 new groups and each group (n=6) were challenged ˜6 months post immunization with one WT dengue challenge serotype. The 2 DEN4 seronegative monkeys were added to a group which was scheduled to be challenge with WT DEN4 virus in order to determine whether these monkeys would be protected in the absence of anti-DEN4 neutralizing antibodies. Protection was defined as the absence/reduction of viremia in immunized monkeys vs. control groups.

All monkeys (6/6) immunized with different tetravalent formulations and challenged with DEN2 or DEN3 WT viruses were completely protected (no detectable viremia), whereas 5/6 animals challenged with DEN1 or DEN4 WT viruses were protected from viremia (Tables 31-35). Nevertheless all animals showed an anamnestic immune response to the challenge virus measured 1 month post challenge (Table 31 and Table 33). Neutralizing data for groups challenged with DEN2 or DEN4 viruses are pending (Table 32 and Table 34).

Considering these data, it can be concluded that a formulation for human trial can include high and equivalent doses of all 4 serotypes (e.g., 5,5,5,5 or 4,4,4,4) or a reduced concentrations of DEN3 and DEN4 chimeras (e.g., 5,5,4,4 or 5,5,3,3). A tetravalent formulation of 4,4,4,4 is currently being filled under cGMP condition and will be tested along with a 5,5,3,3 formulation in cynomolgus monkeys prior to initiation of the upcoming human tetravalent trial.

### II. Toxicology Studies

Two GLP toxicology studies were carried out according to the WHO requirement for testing of YF vaccines at Sierra Biomedical (Nevada, USA). In the first study (Experiment 5) a tetravalent ChimeriVax™ vaccine formulation consisting of 5 logs of each cGMP ChimeriVax™ DEN1-4 viruses (5,5,5,5) stabilized in 10% sorbitol solution was inoculated into cynomolgus monkeys by the IC route. In the second study (Experiment 6), the PMS (WT envelope) and cGMP vaccine (mutant envelope) viruses of ChimeriVax™-DEN1 were inoculated into cynomolgus monkeys by IC route to assure that safety of ChimeriVax™-DEN1 vaccine is not compromised, in a hypothetical situation where mutant envelope is reverted to the WT envelope, in a vaccinated individual.

The in life phase of both studies has been completed and sample analysis is underway. Preliminary unaudited data from the first study is presented below.

### Experiment 5

### A Single-Dose Neurovirulence Study of Tetravalent ChimeriVax-DEN Vaccine Following Intracerebral Administration to Cynomolgus Monkeys

### Objectives

to investigate the potential neurovirulence and acute toxicity of a tetravalent ChimeriVax™-Dengue (DEN) vaccine preparation over a 30-day period following intracerebral administration to cynomolgus monkeys, and
to compare the results from the tetravalent vaccine to the reference article YF-Vax®, the parent vaccine virus that was used to construct the four chimeric yellow fever/dengue virus vaccine strains contained within the tetravalent vaccine.

Twenty-two (11 male and 11 female) experimentally naive, flavivirus-seronegative cynomolgus monkeys (2.0 to 4.2 years of age and weighing 3.2 to 3.8 kg for the males, and 2.1 to 4.6 years of age and weighing 2.2 to 3.0 kg for the females on Day 1) were assigned to treatment groups as shown in Table 36.

All monkeys were dosed via single intracerebral injection on Day 1, observed for 30 days postdose, euthanized, and necropsied.

In life, monkeys were evaluated for clinical signs of toxicity (twice daily) and changes in food consumption (once daily), body weight (approximately weekly), and serum chemistry and hematology parameters (predose on Day 1 and on Days 3, 5, 7, 15, and 31). Clinical signs were assigned scores according to a clinical scoring system, based on the WHO requirements for yellow fever vaccine (WHO 1988). Blood samples were collected on Days 1 (predose) and 2-11 for viral titer analysis, and on Days 1 (predose) and 31 for anti-flavivirus antibody titer analyses.

At necropsy, gross pathologic findings were recorded and tissues were collected and preserved. Slides were prepared from a selected subset of tissues and evaluated by a board-certified SBi pathologist (liver, spleen, heart, kidney, and adrenal glands) or the Sponsor-enlisted neuropathologist (brain and spinal cord). There were no vaccine-related changes in body weight or serum chemistry and hematology parameters.

Possible vaccine-related clinical signs were limited to pupil dilation for five YF-Vax®-treated (Group 1) monkeys and two ChimeriVax™-DEN vaccine-treated (Group 2) monkeys between Days 16 and 22. Two of these Group 1 monkeys also had neurohistologic evidence of encephalitis, although encephalitis was not evident in the remaining three Group 1 monkeys or in Group 2 monkeys; a number of additional monkeys from both groups had evidence of encephalitis with no pupil dilation. Earlier incidence of pupil dilation for two monkeys from each group was considered secondary to trauma from surgery and dose administration.

Vaccine-related changes in clinical scores between Days 16 and 22 were primarily related to the incidence of pupil dilation. Significant intergroup differences in mean clinical scores for on Days 17 and 18 reflected a greater incidence of pupil dilation for Group 1 monkeys.

Vaccine-related gross findings were limited to enlarged lymph nodes or tonsils, and/or accentuated follicular pattern in the spleen or lymphoid tissue at the base of the tongue. Changes in lymphoid organs correlated histologically with hyperplasia of germinal centers and/or paracortex with similar incidence and severity in both vaccine-treated groups. These findings were secondary to the expected immune stimulation induced by the vaccines.

Minimal to moderate lymphoid hyperplasia in one or more lymphoid tissues also occurred at an unusually high frequency with 6/11 Group 1 and 5/11 Group 2 monkeys affected. Affected tissues included spleen, various lymph nodes, tonsils, and lymphoid tissue at the base of the tongue. Lymphoid hyperplasia was considered secondary to immunostimulation in this study.

Lesions in the meninges and the brain/spinal cord matter were scored using a scale of 0-2, where scores were assigned according to the following observations:
grade 0— no visible lesions,
grade 1 (minimal)-1-3 small and/or one large infiltrate, mostly perivascular; a few small foci of more diffuse infiltration, unconnected with blood vessels; and
grade 2 (mild)—more than 3 small and/or 2 or more large perivascular infiltrates; several foci of cellular infiltration, unconnected with blood vessels (some neurons may be involved in these foci of inflammation).

An estimation of the degree of neurovirulence was accomplished using a concept of the target and discriminator areas that was described previously in the WHO requirements for yellow fever vaccine (WHO, 1998). For cynomolgus monkeys, the substantia nigra and cervical and lumbar enlargements of the spinal cord were previously defined as the target formations, whereas basal ganglia and thalamic nuclei were considered as discriminator areas (Levenbook et al., J. Biol. Stand. 15:305-313, 1987). Individual and group mean lesion scores (MLSs) for the target and discriminator areas were calculated separately and as a combined score. Statistical analysis of lesion scores was performed using non-parametric Kruskal-Wallis test for comparison between groups.

Lesions in the meninges and in the brain or spinal cord substance of monkeys from two groups of this study were only inflammatory. The lesions were observed in 9/11 monkeys treated with YF-Vax® virus and in 6/11 monkeys that received tetravalent ChimeriVax™-DEN vaccine virus. Perivascular infiltrates consisted of mononuclear cells with different shapes of nuclei. Rare microglial cell infiltrates could be seen in some cases. Lesions caused by YF-Vax® were scored “1” and “2,” and lesions induced by tetravalent ChimeriVax™-DEN vaccine did not exceed grade “1.” The individual and group MLSs for the target and discriminator areas, as well as combined scores are presented in Table 37 and Table 38.

Yellow fever vaccine virus was detected in the sera of 10/11 monkeys inoculated with YF-Vax®. In monkeys that developed viremia, duration of viremia was 0-4 days post inoculation with individual peak titers ranging from 20 to 860 PFU/mL. ChimeriVax™-DEN1-4 tetravalent vaccine virus, as measured by total viremia, was detected in all monkeys inoculated with ChimeriVax™-DEN1-4. The duration of viremia was 4-9 days post inoculation with individual peak titers ranging from 50 to 2000 PFU/mL. Mean daily viremia levels are illustrated graphically in FIG. 9 and summarized in Table 39.

In this study, peak viremia levels in both groups were below 500 and 100 mouse IC LD₅₀ values [estimated to equal 4.3 log₁₀ and 3.6 log₁₀ Vero cell PFU/mL (Guirakhoo et al., Virology 257:363-372, 1999), respectively for YF-Vax], which are the maximum acceptable titers established under the WHO requirements for yellow fever 17D vaccine (WHO 1998).

Mean peak viremia levels of monkeys inoculated by the IC route with ChimeriVax-DEN1-4 (2.7 log₁₀) were higher than mean peak viremia levels in the YF-Vax® group (2.1 log₁₀). However, they were similar to viremia levels observed in monkeys inoculated subcutaneously with the tetravalent formulation at a dose of 5 log₁₀ of each dengue component (2.6 log₁₀). Moreover, the viremia profiles of a ChimeriVax™-DEN2 cGMP vaccine inoculated into monkeys by the SC route, was similar (mean peak titer of 2.6 log₁₀ PFU/mL and mean duration of 6.3 days, to that described in this study. As described above, ChimeriVax™-DEN2 inoculated into humans was safe and immunogenic.

Viral titers reported above for ChimeriVax™-DEN1-4 are a measurement of total serum viremia and therefore a composite of viremias against all four serotypes contained in the tetravalent vaccine. It is expected that viremia due to any one virus component will be less than the total. This is borne out by SC inoculation studies.

It is concluded that Intracerebral injection of tetravalent ChimeriVax™-DEN vaccine and YFVax® to male and female cynomolgus monkeys was well tolerated at dose levels of ˜1.0×10⁵ TCID₅₀ each for ChimeriVax™-DEN1, ChimeriVax™-DEN2, ChimeriVax™-DEN3, and ChimeriVax™-DEN4 within the tetravalent ChimeriVax™-DEN vaccine and ˜5.5×10⁴ PFU for YF-Vax®. Vaccine-related clinical signs of pupil dilation between Days 16 and 22 were more frequent in the YF-Vax®-treated group, especially on Days 17 and 18, when clinical scores were significantly different between groups. Vaccine-related gross anatomic and histological findings from non-neural tissues were secondary to vaccine-induced immunostimulation.

The neurovirulence of the tetravalent ChimeriVax™-DEN vaccine preparation was minimal, and the target region scores for the group given tetravalent ChimeriVax™-DEN vaccine were significantly lower than the scores for the group treated with yellow fever vaccine (p<0.023). Overall, the tetravalent ChimeriVax™-DEN vaccine induced pupil dilation and neurohistologic signs of encephalitis at lower frequencies than YF-Vax®.

### Experiment 6

### A Single-Dose Neurovirulence Study of ChimeriVax™-Dengue 1 Pre-Master Seed (Clone J-2-P7) and Bulk Vaccines Following Intracerebral Administration to Cynomolgus Monkeys

ChimeriVax-DEN1 vaccine strain became less virulent than PMS virus (determined in suckling mouse and monkey models) during GMP manufacturing in LS10 Vero cells. The attenuation was determined to be due to a single amino acid substitution from K to R at position 204 on the E-protein and occurred first at MS level. The mutation was stable throughout GMP manufacturing of the Vaccine lot (P10) as well as in vitro passages up to P20. The following experiment is designed to measure safety profile of the vaccine in case of an in vivo reversion to WT at E204 residue.

### Objectives:

to investigate and compare any potential neurovirulence and acute toxicity of ChimeriVax™-Dengue 1 Pre-Master Seed (Clone J-2-P7) and ChimeriVax™-Dengue 1 Bulk Vaccines over a 30-day period following intracerebral administration to cynomolgus monkeys, and
to compare the results from the ChimeriVax™-Dengue 1 Pre-Master Seed (Clone J-2-P7) and ChimeriVax™-Dengue 1 Bulk Vaccine tests to YF-Vax® reference. The study was conducted according to good laboratory practices (GLP) standards (21CFR Part 58).

Eighteen cynomolgus monkeys lacking detectable anti-flavivirus antibodies were assigned to treatment groups as is shown in Table 40.

All monkeys were dosed via single intracerebral injection on Day 1, observed for 30 days, then euthanized and necropsied. The monkeys were evaluated for changes in clinical signs (twice daily), body weight (weekly), and food consumption (daily). Clinical signs were assigned scores according to a clinical scoring system, based on the World Health Organization (WHO) requirements for yellow fever vaccine.

Blood samples were collected prestudy and pre-inoculation on Day 1, and on Days 3, 5, 7, 15, and 31 for clinical pathology analysis (serum chemistry and hematology parameters). Additional blood samples were collected pre-inoculation on Day 1 and on Days 2-11 for viral titer analysis, and on Days 1 (predose) and 31 for antiviral antibody titer analyses.

A complete necropsy was performed on Day 31 and tissues collected for preservation. Tissue was prepared for histopathology of the liver, spleen, heart, kidney and adrenal glands. Histopathology of the brain and spinal cord was performed according to the methods described by Levenbook et al., J. Biol. Stand. 15:305-313, 1987, and incorporated into the WHO requirements for yellow fever vaccine.

### Mosquito Transmission: Growth of Chimerivax™-DEN-1,2,3,4 Viruses in Aedes Albopictus (C6/36) Mosquito Cell Culture and in Aedes Aegypti Mosquitoes

The ability of the chimeric dengue vaccine virus tetravalent mix, ChimeriVax™-DEN-1,2,3,4, to replicate in Aedes albopictus mosquito cell culture and in Aedes aegypti mosquitoes, the principle mosquito vector of yellow fever and dengue fever viruses, was evaluated. The growth kinetics of each of the ChimeriVax™-DEN viruses was compared to the yellow fever vaccine17D and corresponding parent wild-type (wt) DEN viruses. Growth kinetics of the ChimeriVax™-DEN-1,2,3,4 tetravalent mix was also explored. Routes of infection in mosquitoes include intrathoracic inoculation (IT) and oral feeding. The replication profile of the chimeric viruses in mosquito tissue and mosquitoes was similar to that of YF 17D virus. Growth of the chimeric viruses in mosquito C6/36 cells was reduced compared to the wt DEN viruses, with the exception of ChimeriVax-DEN4. ChimeriVax-DEN4 replicated to the highest titer compared to the other chimeric viruses, and was similar to that of wt DEN-4 virus. However, replication of all of the chimeric viruses in C6/36 cells was lower than that of YF 17D virus. Interestingly, the growth rate of each chimeric virus was similar whether it was a single serotype infection, or part of the tetravalent mix. No interference by one chimeric virus with a faster growth rate over a slower-growing serotype was observed.

Mosquitoes were intrathoracically inoculated with virus to preclude the potential infection barriers in the midgut associated with oral feeding. The chimeric viruses replicated and disseminated to head tissue, similar to the wild type DEN viruses. Mean titers of the chimeric viruses IT inoculated as a tetravalent mix were in the same range as mean titers in single infections. Peak mean titers of all chimeric viruses were lower than that of IT inoculated YF 17D virus.

The ChimeriVax-DEN viruses infected mosquitoes poorly via infectious blood meals compared to the wild type DEN parent viruses, which indicates that the chimeric viruses are not able to infect and replicate in Ae. aegypti midgut tissue. The results of this study are consistent with previous mosquito vector competence studies using ChimeriVax-JE, ChimeriVax-DEN2 and ChimeriVax-WN viruses (Bhatt et al., Am. J. Trop. Med. Hyg. 62:480-484, 2000; Johnson et al., Am. J. Trop. Med. Hyg. 67:260-265, 2002). YF 17D is a live, attenuated vaccine virus with limited replication activity in mosquito tissue. Previous studies have shown that YF 17D virus is not transmitted by vector mosquito species. The chimeric YF 17D/DEN viruses appear to be further attenuated in mosquitoes. Therefore, it is unlikely that a mosquito would become infected by feeding on a viremic, ChimeriVax™-DEN1,2,3,4 virus vaccine, and there is little potential for transmission of the ChimeriVax-DEN1,2,3,4 viruses by vector mosquitoes.

All references cited herein are incorporated herein by reference.

TABLE 1
Amino acid differences (in the prME region) between
chimeric viruses and their parent wild type viruses.

| | | | | Amino | | |
|---|---|---|---|---|---|---|
| | | Nucleotide | Nucleotide | acid | Amino | |
| ChimeriVax- | Gene | no.^{a} | change | change | acid no.^{b} | Comments |
| DEN1₉₉^{c} | M | 389 | A to G | H to R | 39 | Present in plasmid |
| (VeroP4) | E | 1109 | A to G | K to R | 204 | |
| | E | 1978-80 | CAG to GGC | Q to G | 494 | Created to insert Narl |
| DEN1₀₀ | M | 389 | A to G | H to R | 39 | Present in plasmid |
| (VeroP2) | E | 1978-80 | CAG to GGC | Q to G | 494 | Created to insert Narl |
| DEN3₉₉^{c} | E | 1964 | C to T | A to V | 489 | |
| (VeroP4) | E | 1972-74 | CAA to GGC | Q to G | 492 | Created to insert Narl |
| DEN3₀₀ | PrM | 54 | A/C | E/D | 18 | Likely not present in |
| (VeroP5) | | | | | | mature virions |
| DEN4₉₉^{c} | M | 400 | G to A | A to T | 43 | |
| (VeroP3) | E | 508 | G to A | V to I | 4 | |
| | E | 666 | A to T | L to F | 56 | Created to insert BstBl |
| | E | 1807 | C to T | H to Y | 437 | |
| | | 1978-80 | CAA to GGC | Q to G | 494 | Created to insert Narl |
| DEN4₉₉ | M | 400 | G to A | A to T | 43 | |
| (VeroP8, | E | 666 | A to T | L to F | 56 | Created to insert BstBl |
| large | E | 1327 | C to A | H to N | 277 | |
| plaque)^{c} | E | 1595 | A to G | N to S | 366 | |
| | E | 1807 | C to T | H to Y | 437 | |
| | E | 1977-80 | CAA to GGC | Q to G | 494 | Created to insert Narl |
| DEN4₉₉ | M | 400 | G to A | A to T | 43 | |
| (VeroP8, | E | 666 | A to T | L to F | 56 | Created to insert BstBl |
| small | E | 1977-80 | CAA to GGC | Q to G | 494 | Created to insert Narl |
| plaque)^{c} | | | | | | |
| DEN4₀₀ | E | 1978-80 | CAA to GGC | Q to G | 494 | Created to insert Narl |
| (VeroP5) | | | | | | |

^{a}From beginning of the prME gene.
^{b}From the N-terminus of the proteins (M or E).
^{c}Described by Guirakhoo et al., Virology 290: 309-319, 2001. ChimeriVax-DEN4₉₉ viruses were a mixed population of small to large plaques in Vero cells. Plaques were subjected to 3 rounds of amplification and purification to create two homogeneous populations, small and large sizes at passage 8 (P8) post-transfection. The titers of small and large plaque virus stocks prepared at P8 in Vero cells were 6.9 and 7.1 log₁₀ PFU/ml, respectively.

TABLE 2
Origin and passage history of WT dengue viruses

| Dengue | Strain | Country | Origin | Collection | Passage | |
|---|---|---|---|---|---|---|
| Serotype | Number | of Origin | of Virus | Date | History | Genotype^{a} |
| 1 | PUO-359, | Thailand | Human | 1980 | C6/36 P2 | II? |
| | TVP-1140 | | Plasma | | | |
| 1 | BE H 455823 | Brazil | Human | 1986 | C6/36 P2 | II? |
| | | | Serum | | | |
| 1 | JKT 85-464 | Indonesia | Human | 1985 | C6/36 P2 | I? |
| | | | Serum | | | |
| 2 | PUO-218 | Thailand | Human | 1980 | Mosq P1, | IIIa |
| | | | Plasma | | LLCMK2 P1, | |
| | | | | | Vero P1, | |
| | | | | | C6/36 P2 | |
| 2 | S16803 | Thailand | Human | 1974 | PGMK^{b} P4, | I? |
| | | | | | C6/36 P3 | |
| 2 | JAH | Jamaica | Human | 1982 | C6/36 P2 | IIIb? |
| 2 | PR159 | Puerto Rico | Human | 1969 | Mosq P1, | I |
| | | | | | C6/36 P2 | |
| 3 | PaH881/88 | Thailand | Human | 1988 | AP61 P1, | II |
| | | | | | C6/36 P1 | |
| 3 | 1301 | Malaysia | Human | 1975 | Mosq P2, | I? |
| | | | Serum | | C6/36 P2 | |
| 3 | 1325 | Sri Lanca | Human | 1981 | Mosq P2, | III? |
| | | | Serum | | C6/36 P2 | |
| 4 | 1228 | Indonesia | Human | 1978 | Mosq P2, | II? |
| | (TVP-980) | | Serum | | C6/36 P2 | |
| 4 | BC 26-97 | Mexico | Human | 1996 | C6/36 P3 | II? |
| 4 | P 75-215 | Malaysia | Mosquito | 1975 | ?, C6/36 P2 | I? |

^{a}Genotypes of these viruses have not yet been determined with the exception of the PUO-218 (Lewis et al., Virology 197: 216-224, 1993; Uzcategui et al., J. Gen. Virol. 82: 2945-2953, 2001) and PR159 (Zin et al., S. Asian J. Trop. Med. Public Health 26: 664-668, 1995a; Zin et al., Microbiol. Immunol. 39: 581-590, 1995b) strains of DEN2, and the PaH881/88 strain of DEN3 (Deubel, CABI Publ. New York, pp. 335-365, 1997) viruses. The assumed genotypes (shown with “?”) are based on published information of other strains isolated from similar geographical locations: Den1: Chungue et al., J. Gen. Virol., 76: 1877-1884, 1995; Den2: Lewis et al., Virology 197: 216-224, 1993; Rico-Hesse et al., Am. J. Trop. Med. Hyg. 58: 96-101, 1998; Singh et al., J. Infect. Dis., 180: 959-965, 1999; and Usuku et al., Arch. Virol. 146: 1381-1390, 2001; Den3: Kobayashi et al., Am. J. Trop. Med. Hyg. 60: 904-909, 1999; Den4: Lanciotti et al., J. Gen. Virol. 78: 2279-2290, 1997.
^{b}Primary green monkey kidney cells.

TABLE 3
Nucleotide and amino acid changes in uncloned and cloned PMS candidates of ChimeriVax-DEN1.

| | | | | Nucleotide | Amino | | |
|---|---|---|---|---|---|---|---|
| | | | Nucleotide | change/ | acid change/ | Amino | |
| Candidate | Passage | Gene | No | (heterogeneity) | (heterogeneity) | acid No | Comments |
| Uncloned | P2 | — | — | — | — | — | |
| | P5 | E | 1590 | (A/G) | (K/R) | 204 | f-g loop of domain II |
| | | E | 1730 | (G/T) | (V/F) | 251 | j strand of domain II |
| | P15 | E | 1590 | A to G | K to R | 204 | same as D2 P15 |
| | | E | 1730 | (G/T) | (V/F) | 251 | same as D2-A P20 |
| | | NS4B | 7237 | (A/G) | (I/M) | 113 | same as in D3 uncl. |
| | | NS4B | 7466 | (C/t) | (P/S) | 190 | P15 |
| Clone J | P3 | — | — | — | — | — | |
| | P6 | — | — | — | — | — | |
| | P7 (PMS) | — | — | — | — | — | |
| | P10 | E | 1590 | A to G | K to R | 204 | same as D2 P15 |
| | P20 | E | 1590 | A to G | K to R | 204 | |
| | | NS4B | 6966 | (G/T) | (S/I) | 23 | |
| | | NS4B | 7190 | (G/a) | (V/I) | 98 | |

Bold numbers represent “hot spots” for mutation
— means no change. Uppercase and lowercase letters indicate relative amounts of nucleotides at positions where heterogeneity was observed (N, >50%; n, <50%; N/N, roughly 50%/50%.

TABLE 4
Nucleotide and amino acid changes in uncloned and cloned PMS candidates of ChimeriVax-DEN2.

| | | | | Nucleotide | Amino acid | | |
|---|---|---|---|---|---|---|---|
| | | | Nucleotide | change/ | change/ | Amino | |
| Candidate | Passage | Gene | No | (heterogeneity) | (heterogeneity) | acid No | Comments |
| Uncloned | P2 | — | — | — | — | — | |
| | P15 | prM | 515 | A to C | M to L | 12 | |
| | | E | 1321 | G to A | — | 114 | |
| | | E | 1590 | A to G | K to R | 204 | f-g loop of domain |
| | | NS2A | 4046 | A to T | I to L | 176 | II; as in D1 |
| | | NS3 | 5485 | T to C | — | 301 | same as in D3-A |
| | | 2K | 6888 | C to T | A to V | 20 | P20 |
| | | NS4B | 7427 | C to T | L to F | 177 | |
| | | NS5 | 9094 | (G/T) | — | 482 | |
| | | NS5 | 10307 | C to T | — | 887 | |
| Clone A | P6 | — | — | — | — | — | |
| | P7 (PMS) | — | — | — | — | — | |
| | P10 | prM | 551 | T to G | L to V | 24 | |
| | | E | 1730 | G to T | V to F | 251 | j strand of |
| | | | | | | | domainII; as in D1 |
| | P20 | prM | 551 | T to G | L to V | 24 | |
| | | E | 1730 | G to T | V to F | 251 | j strand of |
| | | | | | | | domainII; as in D1 |
| Clone B | P6 | E | 1030 | A to G | — | 17 | Introduced by |
| | | E | 1789 | C to T | — | 270 | YFpol |
| | | | | | | | introduced by |
| | | | | | | | YFpol |
| | P15 | E | 1030 | A to G | — | 17 | same as in P6 |
| | | E | 1590 | A to G | K to R | 204 | f-g loop of domain |
| | | E | 1789 | C to T | — | 270 | II; as in D1 |
| | | E | 2444 | T to C | — | 489 | same as in P6 |
| | | NS4B | 7443 | C to T | A to V | 182 | |
| | | NS5 | 8677 | A to G | — | 343 | |

Bold numbers represent “hot spots” for mutation.
— means no change.
Uppercase and lowercase letters indicate relative amounts of nucleotides at positions where heterogeneity was observed (N, >50%; n, <50%; N/N, roughly 50%/50%).

TABLE 5
Nucleotide and amino acid changes in uncloned and cloned PMS candidates of ChimeriVax-DEN3.

| | | | | Nucleotide | Amino acid | | |
|---|---|---|---|---|---|---|---|
| | | | Nucleotide | change/ | change/ | Amino | |
| Candidate | Passage | Gene | No | (heterogeneity) | (heterogeneity) | acid No | Comments |
| Uncloned | P2 | — | — | — | — | — | |
| | P5 | — | — | — | — | — | |
| | P10 | prM | 564 | C to A | A to D | 28 | |
| | | NS1 | 2561 | T to C | Y to H | 35 | |
| | P15 | prM | 564 | C to A | A to D | 28 | |
| | | NS1 | 2561 | T to C | Y to H | 35 | |
| | | NS4A | 6502 | (a/G) | — | 19 | |
| | | NS4B | 7231 | (a/G) | (I/M) | 113 | same as in D1 uncl. P15 |
| Clone A | P6 | NS4A | 6607 | C to T | — | 54 | |
| | P7 (PMS) | NS4A | 6607 | C to T | — | 54 | |
| | P10 | NS4A | 6607 | C to T | — | 54 | |
| | P15 | NS4A | 6607 | C to T | — | 54 | |
| | | NS5 | 7859 | C/t | | 73 | |
| | P20 | NS4A | 6607 | C to T | — | 54 | |
| | | NS4B | 7421 | C to T | L to F | 177 | same as in D2 uncl. |
| | | NS5 | 7859 | (C/T) | — | 73 | P15 |

Bold numbers represent “hot spots” for mutations means no change.
Uppercase and lowercase letters indicate relative amounts of nucleotides at positions where heterogeneity was observed (N, >50%; n, <50%; N/N, roughly 50%/50%).

TABLE 6
Sequencing results of the uncloned and cloned PMS candidates of ChimeriVax-DEN4.

| | | | | Nucleotide | Amino acid | | |
|---|---|---|---|---|---|---|---|
| | | | Nucleotide | change/ | change/ | Amino | |
| Candidate | Passage | Gene | No | (heterogeneity) | (heterogeneity) | acid No | Comments |
| Uncloned | P2 | — | — | — | — | — | |
| | P15 | — | — | — | — | — | |
| Clone B | P6 | — | — | — | — | — | |
| | P7 (PMS) | — | — | — | — | — | |
| | P10 | — | — | — | — | — | |
| | P20 | — | — | — | — | — | |

— means no change.

TABLE 7
Genomic stability of ChimeriVax ™-DEN1-4 vaccine
viruses during cGMP production in LS10 Vero cells

| | PMS (P7) | | MS (P8) | | WS (P9) | | VL (P10) | |
|---|---|---|---|---|---|---|---|---|
| | NT | AA | NT | AA | NT | AA | NT | AA |
| ChimeriVax- | change | change | change | change | change | change | change | change |
| DEN1 | — | — | 1590 | E204 | 1590 | E204 | 1590 | E204 |
| | | | a/G | K/R | A > G | K > R | A > G | K > R |
| DEN2 | — | — | 551 | PrM24 | 551 | PrM24 | 551 | PrM24 |
| | | | t/G | L/V | T > G | L > V | T > G | L > V |
| | | | 1730 | E251 | 1730 | E251 | 1730 | E251 V > F |
| | | | G/t | V/F | g/T | V/F | G > T | |
| DEN3 | NS4a | — | NS4a | — | N | N | NS4a | — |
| | 6607 | | 6607 | | D | D | 6607 | |
| | C > T | | C > T | | | | C > T | |
| DEN4 | — | — | — | — | N | N | — | — |
| | | | | | D | D | | |

Uppercase and lowercase letters indicate relative amounts of nucleotides at positions where heterogeneity was observed (N, >50%; n, <50%; N/N, roughly 50%/50%)
ND: Not done.
—: No change.

TABLE 8
Neurovirulence of various clones of ChimeriVax ™-DEN1
viruses in 4-day old infant mice.

| | | | | No dead/ | |
|---|---|---|---|---|---|
| ChimeriVax- | | | Dose | total | AST |
| DEN1 | AA change | Dilution | (BT) | (% dead) | Days |
| Uncloned | None | Neat | 5.0 | 11/11 (100) | 9.1 |
| | | 1:10 | 4.1 | 11/11 (100) | 10.2 |
| Clone B | E251 (V > F) | Neat | 5.8 | 10/11 (91) | 9.8 |
| | | 1:10 | 5.0 | 11/11 (100) | 10.2 |
| Clone C | E311 (E > D) | Neat | 5.8 | 11/11 (100) | 8.5 |
| | E351 (V > L) | 1:10 | 4.9 | 11/11 (100) | 9.5 |
| Clone E | E204 (K > R) | Neat | 5.9 | 3/11 (27) | 13 |
| | | 1:10 | 4.8 | 1/11 (9) | 14 |
| | | 1:100 | 4.0 | 1/11 (9) | 15 |
| Clone J | None | Neat | 3.6 | 11/11 (100) | 10.8 |
| | | 1:10 | 3.0 | 11/11 (100) | 11.3 |
| | | 1:100 | 1.8 | 9/11 (82) | 11.3 |
| YF-VAX | NA | 1:20 | 2.5 | 12/12 (100) | 8.3 |

TABLE 9
Neurovirulence of ChimeriVax-DEN1-4 viruses [PMS (P7), GMP MS (P8), P9 and P10] in 4-day-old mice

| | | | | | | | LD₅₀ |
|---|---|---|---|---|---|---|---|
| CV- | Passage^{a} | AA Changes^{f} | Titer^{b} | Dilution | Dose (BT)^{c} | Mortality | Log₁₀PFU |
| DEN1 | P7 (PMS) | None | 6.5 | 10⁻², 10⁻³ | 3.1, 2.1 | 10/11, 8/11 | <2.1 |
| | P8 (_{GMP}MS) | E204K > R | 6.7 | 10⁻¹, 10⁻² | 2.6, 1.6 | 7/11, 8/11 | <1.6 |
| | P9 | ND | 7.8 | 10⁻², 10⁻³ | 4.1, 3.1 | 2/11, 3/11 | >4.1 |
| | P10 | E204K > R | 7.9 | 10⁻¹, 10⁻², 10⁻³ | 5.1, 4.1, 3.1 | 1/13, 1/13, 0/12 | >5.1 |
| DEN2 | P7 (PMS) | None | 6.9 | 10⁻², 10⁻³ | 3.3, 2.3 | 11/11, 8/11 | <2.3 |
| | P8 (_{GMP}MS) | PrM24L/V, E251 V/F | 5.6 | 10⁻¹, 10⁻² | 2.0, 1.0 | 11/11, 9/11 | <1.0 |
| | aysP10□None | ND | 7.4 | 10⁻², 10⁻³ | 3.8, 2.8 | 11/12, 10/12 | <2.8 |
| | P10 | PrM24L > V, E251V > F | 7.7 | 10⁻², 10⁻³, 10⁻⁴ | 4.0, 3.0, 2.0 | 11/12, 10/13, 5/13 | 2.7 |
| DEN3 | P7 (PMS) | None | 6.1 | Neat | 3.6 | 4/12 | >3.6 |
| | P8 (_{GMP}MS) | None | 5.0 | Neat | 3.5 | 3/12 | >3.5 |
| | P9 | ND | 5.8 | Neat, 10⁻¹ | 4.0, 3.0 | 0/13, 0/13 | >4.0 |
| | P10 | E202K/R | 6.7 | Neat, 10⁻¹ | 3.7, 2.7 | 0/13, 0/13 | >3.7 |
| DEN4 | P7 (PMS) | None | 6.4 | 10⁻², 10⁻³ | 2.3, 1.3 | 9/11, 6/11 | 2.2 |
| | P8 (_{GMP}MS) | None | 4.8 | 10⁻¹, 10⁻² | 2.5, 1.5 | 11/12, 9/11 | <1.5 |
| | P9 | ND | 6.0 | 10⁻², 10⁻³ | 2.4, 1.4 | 8/11, 1/12 | 1.9 |
| | P10 | None | 7.0 | 10⁻¹, 10⁻², 10⁻³ | 4.0, 3.0, 2.0 | 12/13, 7/13, 4/13 | 2.4 |
| YF-VAX^{d} | Unpassaged | NA | 2.2 | 1:40 | 2.2 | 15/15 | <2.2 |
| YF-VAX^{e} | Unpassaged | NA | 2.2 | 1:40 | 1.3 | 13/13 | <1.3 |

^{a}P9 and P10 viruses were produced in research laboratory by 2 passages of _{c}GMP MS (P8) in Vero (LS10) cells.
^{b}log₁₀ PFU/ml.
^{c}Doses were determined by back titration (BT) of inocula.
^{e}Used as a control for experiment 1 (P7, P8, and P9 viruses).
^{d}Used as a control for experiment 2 (P10 viruses).
^{f}No silent mutation was found in any viruses except for DEN3 (one nucleotide change in NS4A6607, which did not result in AA substitution, was found in all passages, P7-P10, of DEN3 chimeras).
NA: Not applicable. Bold numbers indicate significant reduction in neurovirulence.

TABLE 10
Neurovirulence of PMS (P7) and P20 of ChimeriVax-DEN1-4 viruses in 4-day-old mice.

| | | AA changes | | | | Plaque | | LD₅₀ |
|---|---|---|---|---|---|---|---|---|
| CV- | Passage | (heterogeneity) | Titer^{e} | Dilution | Dose (BT)^{f} | size (mm) | Mortality | Log₁₀ PFU |
| DEN1 | P7 (PMS) | None | 6.5 | 10⁻², 10⁻³ | 2.9, 1.9 | ~1-1.5 | 10/10, 8/10 | <1.9 |
| | P20 | E204K > R | 7.8 | 10⁻², 10⁻³ | 3.8, 2.8 | ~3-5^{b} | 0/9, 0/9 | >3.8 |
| | | NS4b23 (S/I) | | | | | | |
| | | NS4b98(V/I) | | | | | | |
| DEN2 | P7 (PMS) | None | 6.9 | 10⁻², 10⁻³, 10⁻⁴ | 3.3, 2.3, 1.3 | ~1 | 10/10, 8/10, 6/10 | 1.3 |
| | P20 | PrM24L > V | 7.8 | 10⁻², 10⁻³, 10⁻⁴ | 3.6, 2.6, 1.6 | 1.5-2^{c} | 7/9, 4/10, 2/9 | 3.5 |
| | | E251V > F | | | | | | |
| DEN3^{a} | P7 (PMS) | None | 6.1 | Neat, 10⁻¹ | 4.6, 3.6 | ~1-1.5 | 2/10, 2/10 | >4.6 |
| | P20 | NS4b177L > F | 6.4 | Neat, 10⁻¹ | 4.1, 3.1 | ~1-1.5 | 0/10, 0/10 | >4.1 |
| DEN4^{a} | P7 (PMS) | None | 6.4 | 10⁻², 10⁻³, 10⁻⁴ | 2.6, 1.6, 0.6 | ~1 | 4/11, 3/11, 0/11 | >2.6 |
| | P20 | None | 7.6 | 10⁻², 10⁻³, 10⁻⁴ | 3.7, 2.7, 1.7 | ~2.5^{d} | 2/11, 1/11, 0/11 | >3.7 |
| YF-VAX | None | NA | ~5.2 | 1:40 | 2.2 | | 9/9 | <2.2 |

^{a}This is a repeated test due to mislabeling of DEN3 and DEN4 cages.
^{b}P10 plaques = ~3 mm.
^{c}P10 plaques = ~2 mm.
^{d}P10 plaques = 2 mm.
^{e}(log₁₀ PFU/ml).
^{f}Dose was determined by back titration of inocula. Bold numbers show a significant reduction in neurovirulence.

TABLE 11
Infant Mouse Neurovirulence Test of P10 (Vaccine Level)
viruses as Monovalent or Tetravalent Formulations

| | | Titer | | | | |
|---|---|---|---|---|---|---|
| | | (log₁₀ | Dose (log₁₀ | Volume | Mortality | AST |
| ChimeriVax- | Passage | PFU/ml) | PFU)/BT | IC (ml) | (%) | (Days) |
| DEN1 | P10 | 7.9 | 2.0/1.7 | 0.02 | 4/9 (44) | 13.7 |
| DEN2 | P10 | 7.7 | 2.0/1.8 | 0.02 | 2/9 (22) | 14 |
| DEN3 | P10 | 6.7 | 2.0/1.3 | 0.02 | 0/10 (0) | NA |
| DEN4 | P10 | 7.0 | 2.0/1.4 | 0.02 | 3/9 (33) | 13.3 |
| DEN1 + DEN2 + | P10 | 7.9, 7.7, | 2.0 logs each, | 0.02 | 3/9 (33) | 14.7 |
| DEN3 + DEN4 | | 6.7, 7.0 | 2.6 logs total/ND | | | |
| YF-VAX | None | ~5.2 | ~2.0/2.2 | 0.02 | 9/9 (100) | 7.7 |

ND: Not done,
NA: Not applicable

TABLE 12
Viremia in rhesus monkeys immunized s.c. with ChimeriVax-DEN monovalent vaccine candidates or YF-VAX ®.

| | | Virus (dose, | Viremia (log₁₀ PFU/ml) by day post immunization: _Peak titer^{c} | | | | | | | | | | | | Peak | Duration |
|---|---|---|---|---|---|---|---|---|---|---|---|---|---|---|---|---|
| Monkey | Group | log₁₀ PFU)^{b} | 1 | 2 | 3 | 4 | 5 | 6 | 7 | 8 | 9 | 10 | 11 | 12 | titer^{c} | (Days) |
| R14237M | 1 | ChimeriVax- | 1.0 | 1.4 | 0^{a} | 2.1 | 1.7 | 0 | 0 | 0 | 0 | 0 | 0 | 0 | 2.0 | 3.7 |
| R14256F | | DEN1₉₉ | 1.0 | 2.1 | 0 | 0 | 0 | 0 | 0 | 0 | 0 | 0 | 0 | 0 | | |
| R14243M | | (5.2) | 1.4 | 1.4 | 0.7 | 1.9 | 1.4 | 0 | 0 | 0 | 0 | 0 | 0 | 0 | | |
| R14266F | | | 0 | 0 | 0 | 0 | 0 | 0 | 0 | 0 | 0 | 0 | 0 | 0 | | |
| R14206M | 2 | ChimeriVax- | 0 | 0 | 2.3 | 0 | 1.7 | 1.0 | 0 | 0 | 0 | 0 | 0 | 0 | 1.8 | 3.7 |
| R14282F | | DEN3₉₉ | 0.7 | 1.4 | 1.4 | 1.0 | 0 | 0 | 1.4 | 1.4 | 2.1 | 0 | 0 | 0 | | |
| R14233M | | (4.4) | 0 | 0 | 0 | 0 | 0 | 1.0 | 1.0 | 1.4 | 0 | 0.7 | 0 | 0 | | |
| R14284F | | | 0 | 0 | 1.4 | 0 | 0 | 0 | 0 | 0 | 0 | 0 | 0 | 0 | | |
| R14208M | 3 | ChimeriVax- | 0 | 0 | 0 | 0 | 0 | 0 | 0 | 0 | 0 | 0 | 0 | 0 | 2.1 | 6.3 |
| R14287F | | DEN3₀₀ | 0 | 0 | 1.4 | 1.0 | 0.7 | 1.9 | 0 | 0 | 0 | 1.3 | 1.4 | 0 | | |
| R14235M | | (3.9) | 0 | 0 | 2.1 | 1.7 | 1.9 | 1.0 | 1.0 | 1.4 | 1.9 | 2.1 | 2.5 | 1.7 | | |
| R14277F | | | 0 | 0 | 0 | 0 | 0 | 1.5 | 1.0 | 1.9 | 0 | 0 | 0 | 0 | | |
| R14240M | 4 | ChimeriVax- | 0 | 0 | 0 | 0 | 1.0 | 0 | 0 | 0 | 2.5 | 2.6 | 2.6 | 2.2 | 2.4 | 7 |
| R14257F | | DEN4₉₉, | 1.0 | 1.3 | 0.7 | 0.7 | 1.7 | 1.4 | 1.4 | 1.9 | 2.1 | 2.0 | 0 | 0 | | |
| R14201M | | large plaque (5.0) | 0 | 0.7 | 1.0 | 0 | 2.4 | 2.2 | 0 | 2.3 | 2.2 | 0 | 0 | 0 | | |
| R14252F | 5 | ChimeriVax- | 0 | 0 | 0 | 0 | 0 | 0 | 0 | 0 | 0 | 0 | 0 | 0 | 1.9 | 7 |
| R14238M | | DEN4₉₉, | 0 | 0 | 1.0 | 0 | 1.0 | 1.9 | 1.0 | 1.7 | 1.7 | 1.4 | 0 | 0 | | |
| R14272F | | small plaque (4.3) | 0 | 0 | 0 | 0 | 0 | 0 | 0 | 0 | 0 | 0 | 0 | 0 | | |
| R14241M | 6 | ChimeriVax- | 1.7 | 2.6 | 2.4 | 2.9 | 2.9 | 2.4 | 0 | 0 | 0 | 0 | 0 | 0 | 2.7 | 5.7 |
| R14253F | | DEN4₀₀ | 0 | 2.1 | 2.8 | 2.8 | 2.5 | 0 | 0 | 0 | 0 | 0 | 0 | 0 | | |
| R14242M | | (4.8) | 2.1 | 2.8 | 2.9 | 2.5 | 0 | 0 | 0 | 0 | 0 | 0 | 0 | 0 | | |
| R14273F | | | 0 | 1.7 | 2.2 | 2.0 | 1.4 | 1.7 | 1.0 | 1.9 | 1.0 | 1.4 | 0 | 0 | | |
| R14234M | 7 | YF-VAX ® | 1.0 | 1.4 | 2.0 | 2.3 | 0 | 0 | 0 | 0 | 0 | 0 | 0 | 0 | 2.3 | 3.7 |
| R14281F | | (5.5) | 1.3 | 1.7 | 1.4 | 2.2 | 0 | 0 | 0 | 0 | 0 | 0 | 0 | 0 | | |
| R14214M | | | 1.4 | 1.3 | 1.4 | 1.7 | 0 | 0 | 0 | 0 | 0 | 0 | 0 | 0 | | |
| R14276F | | | 0 | 2.5 | 3.0 | 2.8 | 0 | 0 | 0 | 0 | 0 | 0 | 0 | 0 | | |

^{a}<0.7 log₁₀ PFU/ml.
^{b}Determined by back titration of inocula.
^{c}PFU/ml.

TABLE 13
Neutralizing antibody titers (Day 30)^{a} against homologous
viruses in monkeys immunized with one dose of monovalent
viruses (Group 1 to 6) or YF-VAX (Group 7).

| Monkey | Group | Titer |
|---|---|---|
| R14237M | 1 | 1280 |
| R14256F | | 1280 |
| R14243M | | 640 |
| R14266F | | 1280 |
| GMT | | 1076 |
| R14206M | 2 | 640 |
| R14282F | | 1280 |
| R14233M | | 320 |
| R14284F | | 640 |
| GMT | | 640 |
| R14208M | 3 | 1280 |
| R14287F | | 2560 |
| R14235M | | 640 |
| R14277F | | 1280 |
| GMT | | 1280 |
| R14240M | 4 | 2560 |
| R14257F | | 5120 |
| R14201M | | 1280 |
| GMT | | 2560 |
| R14252F | 5 | 1280 |
| R14238M | | 640 |
| R14272F | | 640 |
| GMT | | 806 |
| R14241M | 6 | 320 |
| R14253F | | 1280 |
| R14242M | | 640 |
| R14273F | | 1280 |
| GMT | | 761 |
| R14234M | 7 | 1280 |
| R14281F | | 1280 |
| R14214M | | 2560 |
| R14276F | | 2560 |
| GMT | | 1810 |

TABLE 14
Viremia in rhesus monkeys immunized s.c. with WT or ChimeriVax-DEN tetravalent vaccine candidates.

| | | Virus | | | | | | | | | | | | | | |
|---|---|---|---|---|---|---|---|---|---|---|---|---|---|---|---|---|
| | | (dose, | | | | | | | | | | | | | | |
| | | log₁₀ | Viremia (log₁₀ PFU/ml) by day post immunization: | | | | | | | | | | | | Peak | Duration |
| Monkey | Group | PFU)^{a} | 1 | 2 | 3 | 4 | 5 | 6 | 7 | 8 | 9 | 10 | 11 | 12 | Titer^{b} | (Days) |
| R14226M | 8 | WT DEN^{d} | <1.7 | 2.7 | 2.3 | 2.2 | 2.3 | 3.5 | 3.1 | 3.5 | 3.4 | 1.7 | <1.7 | <1.7 | 4.3 | 8.5 |
| R14280F | | (D1 = 4.4, | <1.7 | 2.2 | 2.6 | 3.7 | 4.8 | 5.0 | 4.2 | 3.3 | 1.7 | <1.7 | <1.7 | <1.7 | | |
| R14230M | | D2 = 4.0, | <1.7 | <1.7 | 3.3 | 3.4 | 3.2 | 3.9 | 4.3 | 4.7 | 3.2 | 2.7 | <1.7 | <1.7 | | |
| R14271F | | D3 = 5.4, | <1.7 | 2.7 | 3.0 | 3.1 | 3.6 | 4.0 | 4.1 | 3.4 | 2.6 | 2.3 | <1.7 | <1.7 | | |
| | | D4 = 4.8) | | | | | | | | | | | | | | |
| R14207M | 9 | ChimeriVax- | 1.7 | 3.2 | 2.5 | 2.3 | 1.7 | 2.4 | 2.1 | 2.3 | 2.5 | 2.7 | 1.6 | 1.4 | 2.8 | 6^{c} |
| R14286F | | DEN | 2.6 | 3.2 | 2.7 | 1.9 | <1.0 | <1.0 | <1.0 | <1.0 | <1.0 | <1.0 | <1.0 | <1.0 | | |
| R14210M | | (YF/D1 = 4.5 | 1.3 | 2.2 | 2.0 | 1.7 | 2.1 | 1.4 | <1.0 | 2.0 | 1.7 | <1.0 | <1.0 | <1.0 | | |
| R14265F | | YF/D2 = 3.0 | 1.6 | 1.7 | 2.1 | 2.2 | 2.6 | <1.0 | <1.0 | <1.0 | <1.0 | <1.0 | <1.0 | <1.0 | | |
| R14244M | | YF/D3 = 3.6 | 1.0 | 1.9 | 2.6 | 2.6 | 3.1 | 2.8 | 2.2 | 1.7 | <1.0 | <1.0 | <1.0 | <1.0 | | |
| R14269F | | YF/D4 = 4.4) | 2.3 | 2.4 | 2.2 | 1.9 | 2.3 | <1.0 | <1.0 | <1.0 | <1.0 | <1.0 | <1.0 | <1.0 | | |

^{a}Determined by back titration of inocula.
^{b}PFU/ml.
^{c}Days with viremia below 1.7 PFU/ml are not calculated to be comparable to the wild-type group 8.
^{d}D1; PUO-359, C6/36 P2. D2; S16803, PGMK P4, C6/36 P3., D3; PaH881, AP61 P1, C6/36 P2., D4; 1228, mosquito P2, C6/36 P2 (see also Table 1).

TABLE 15
Serotype specific viremia in monkeys immunized with tetravalent
WT dengue (A) or ChimeriVax-DEN (B) viruses.
A

| | Serotype | Viremia (log₁₀ PFU/ml) by day post immunization: | | | | | | | | | | | Peak | Duration |
|---|---|---|---|---|---|---|---|---|---|---|---|---|---|---|
| Monkey | detected | 1 | 2 | 3 | 4 | 5 | 6 | 7 | 8 | 9 | 10 | 11 | Titer | (days) |
| R14226M | D1 | 0^{a} | 1.7 | 2.3 | 2.2 | 2.3 | 3.2 | 3.0 | 3.1 | 3.0 | 0 | 0 | 3.2 | 8 |
| | D2 | 0 | 0 | 0 | 0 | 0 | 2.7 | 2.7 | 2.7 | 3.2 | 1.7 | 0 | 3.2 | 5 |
| | D3 | 0 | 2.7 | 0 | 2.0 | 0 | 3.0 | 2.6 | 2.7 | 0 | 0 | 0 | 3.0 | 5 |
| | D4 | 0 | 0 | 0 | 0 | 0 | 0 | 0 | 0 | 0 | 0 | 0 | 0 | 0 |
| R14280F | D1 | 0 | 0 | 2.0 | 2.0 | 2.7 | 3.8 | 3.5 | 3.3 | 0 | 0 | 0 | 3.8 | 7 |
| | D2 | 0 | 0 | 2.0 | 3.7 | 4.8 | 5.0 | 4.2 | 0 | 0 | 0 | 0 | 5.0 | 5 |
| | D3 | 0 | 2.0 | 2.3 | 2.0 | 2.3 | 2.5 | 2.7 | 0 | 0 | 0 | 0 | 2.7 | 6 |
| | D4 | 0 | 1.7 | 1.7 | 2.7 | 3.0 | 2.7 | 0 | 0 | 0 | 0 | 0 | 2.7 | 5 |
| R14230M | D1 | 0 | 0 | 2.7 | 2.6 | 2.7 | 3.5 | 3.5 | 3.3 | 0 | 0 | 0 | 3.5 | 6 |
| | D2 | 0 | 0 | 2.4 | 3.2 | 3.0 | 3.6 | 4.2 | 4.7 | 3.2 | 2.7 | 0 | 4.7 | 8 |
| | D3 | 0 | 0 | 3.0 | 2.8 | 1.7 | 2.2 | 1.7 | 0 | 0 | 0 | 0 | 3.0 | 5 |
| | D4 | 0 | 0 | 2.5 | 1.7 | 1.7 | 1.7 | 0 | 0 | 0 | 0 | 0 | 2.5 | 4 |
| R14271F | D1 | 0 | 0 | 0 | 2.3 | 2.5 | 3.0 | 3.2 | 2.6 | 2.5 | 2.2 | 0 | 3.2 | 7 |
| | D2 | 0 | 0 | 1.7 | 2.7 | 3.6 | 3.9 | 4.6 | 3.3 | 2.0 | 1.7 | 0 | 4.6 | 8 |
| | D3 | 0 | 2.7 | 3.0 | 0 | 0 | 2.7 | 0 | 0 | 1.7 | 0 | 0 | 3.0 | 4 |
| | D4 | 0 | 0 | 0 | 0 | 0 | 2.0 | 2.7 | 0 | 0 | 0 | 0 | 2.7 | 2 |

^{a}<1.7 log₁₀ PFU/ml

B

| | Serotype | Viremia (log₁₀ PFU/ml) by day post immunization: | | | | | | | | | | | Peak | Duration^{b} |
|---|---|---|---|---|---|---|---|---|---|---|---|---|---|---|
| Monkey | detected | 1 | 2 | 3 | 4 | 5 | 6 | 7 | 8 | 9 | 10 | 11 | Titer | (days) |
| R14207M | D1 | 0^{a} | 2.4 | 0 | 1.4 | 0 | 0 | 0 | 0 | 0 | 0 | 0 | 2.4 | 1 |
| | D2 | 0 | 0 | 0 | 0 | 0 | 0 | 1.4 | 1.4 | 1.7 | 2.7 | 0 | 2.7 | 2 |
| | D3 | 0 | 0 | 0 | 0 | 0 | 0 | 0 | 0 | 0 | 0 | 0 | 0 | 0 |
| | D4 | 0 | 2.0 | 1.7 | 1.4 | 1.4 | 1.4 | 1.7 | 1.9 | 2.7 | 1.4 | 0 | 2.7 | 5 |
| R14286F | D1 | 0 | 2.0 | 0 | 0 | 0 | 0 | 0 | 0 | 0 | 0 | 0 | 2.0 | 1 |
| | D2 | 0 | 0 | 0 | 0 | 0 | 0 | 0 | 0 | 0 | 0 | 0 | 0 | 0 |
| | D3 | 0 | 0 | 0 | 0 | 0 | 0 | 0 | 0 | 0 | 0 | 0 | 0 | 0 |
| | D4 | 1.7 | 2.7 | 2.5 | 2.1 | 0 | 0 | 0 | 0 | 0 | 0 | 0 | 2.7 | 4 |
| R14210M | D1 | 0 | 0 | 0 | 0 | 0 | 0 | 0 | 0 | 0 | 0 | 0 | 0 | 0 |
| | D2 | 0 | 0 | 0 | 0 | 0 | 0 | 0 | 0 | 0 | 0 | 0 | 0 | 0 |
| | D3 | 0 | 0 | 0 | 0 | 0 | 0 | 0 | 0 | 0 | 0 | 0 | 0 | 0 |
| | D4 | 0 | 1.7 | 1.4 | 1.4 | 0 | 1.7 | 0 | 1.7 | 0 | 1.4 | 0 | 1.7 | 3 |
| R14265F | D1 | 0 | 0 | 0 | 0 | 0 | 0 | 0 | 0 | 0 | 0 | 0 | 0 | 0 |
| | D2 | 0 | 0 | 0 | 0 | 0 | 0 | 0 | 0 | 0 | 0 | 0 | 0 | 0 |
| | D3 | ct1 | 0 | 0 | 0 | 0 | 0 | 0 | 0 | 0 | 0 | 0 | 0 | 0 |
| | D4 | 1.7 | 2.3 | 1.9 | 1.4 | 2.0 | 0 | 0 | 0 | 0 | 0 | 0 | 2.3 | 4 |
| R14244M | D1 | 0 | 0 | 0 | 0 | 0 | 0 | 0 | 0 | 0 | 0 | 0 | 0 | 0 |
| | D2 | 0 | 0 | 0 | 0 | 0 | 0 | 0 | 0 | 0 | 0 | 0 | 0 | 0 |
| | D3 | 0 | 0 | 0 | 0 | 0 | 0 | 0 | 0 | 0 | 0 | 0 | 0 | 0 |
| | D4 | 1.7 | 2.3 | 1.9 | 1.4 | 2.0 | 0 | 0 | 0 | 0 | 0 | 0 | 2.3 | 4 |
| R14269F | D1 | 0 | 0 | 0 | 0 | 0 | 0 | 0 | 0 | 0 | 0 | 0 | 0 | 0 |
| | D2 | 0 | 0 | 0 | 0 | 0 | 0 | 0 | 0 | 0 | 0 | 0 | 0 | 0 |
| | D3 | 0 | 0 | 0 | 0 | 0 | 0 | 0 | 0 | 0 | 0 | 0 | 0 | 0 |
| | D4 | 0 | 1.9 | 1.9 | 0 | 2.4 | 2.2 | 2.2 | 1.9 | 0 | 0 | 0 | 2.4 | 6 |

^{a}<1.4 log₁₀ PFU/ml.
^{b}Days with peak titers lower than 1.7 logs are not counted to be comparable to Table A.

TABLE 16
Neutralizing antibody titers (Day 30)^{a}, against homologous
viruses, in monkeys immunized with one dose of tetravalent-WT
(Group 8) or -chimeric (Group 9) viruses.

| | | 50% Neutralizing titers (Day 30) | | | |
|---|---|---|---|---|---|
| | | against homologous viruses^{b} | | | |
| Monkey | Group | DEN1 | DEN2 | DEN3 | DEN4 |
| R14226M | 8 | 320 | 320 | 640 | 160 |
| R14280F | | 1280 | 2560 | 640 | 320 |
| R14230M | | 2560 | >5120 | 2560 | >5120 |
| R14271F | | 1280 | >5120 | >5120 | 1280 |
| GMT | | 1076 | 2153 | >1522 | >761 |
| R14207M | 9 | 2560 | 10240 | 320 | 1280 |
| R14286F | | 640 | 160 | 160 | 1280 |
| R14210M | | 80 | 80 | 80 | 640 |
| R14265F | | 320 | 320 | 320 | 2560 |
| R14244M | | 160 | 640 | 320 | 2560 |
| R14269F | | 320 | 160 | 640 | 1280 |
| GMT | | 359 | 403 | 254 | 1437 |

^{a}Titers of all sera taken immediately before vaccination were <10 and are not shown in this Table.
^{b}For neutralization of Group 8 sera WT parent DEN1-4 and for neutralization of Group 9 sera chimeric DEN 1-4 viruses were used. Bold numbers are Geometric Mean Titers of 50% neutralization titers.

TABLE 17
Neutralizing antibody titers of Group 9 monkeys against homologous (chimeric DEN)
and heterologous (WT DEN isolates) viruses before and after the second dose.

| | 50% neutralizing antibody titers against | | | | | | | | | | | | | | |
|---|---|---|---|---|---|---|---|---|---|---|---|---|---|---|---|
| | indicated dengue viruses by monkey: | | | | | | | | | | | | | | |
| | R14207 | | R14286 | | R14210 | | R14265 | | R14244 | | R14269 | | GMT | | P |
| Virus | Pre^{a} | Post^{b} | Pre | Post | Pre | Post | Pre | Post | Pre | Post | Pre | Post | Pre | Post | Value^{c} |
| YF/DEN1 | 1280 | 5120 | 1280 | 5120 | 320 | 640 | 2560 | 2560 | 160 | 640 | 320 | 1280 | 640 | 1810 | 0.12 |
| PUO359 | 160 | 320 | 160 | 320 | 160 | 160 | 320 | 640 | 80 | 160 | 40 | 80 | 127 | 226 | 0.19 |
| BrazilBEH455823 | 640 | 640 | 320 | 640 | 320 | 320 | 1280 | 2560 | 320 | 320 | 160 | 320 | 403 | 570 | 0.47 |
| Jakarta85-464 | 160 | 320 | 320 | 640 | 160 | 160 | 640 | 1280 | 80 | 320 | 80 | 320 | 80 | 403 | 0.19 |
| YF/DEN2 | 5120 | 5120 | 1280 | 1280 | 640 | 1280 | 1280 | 1280 | 2560 | 2560 | 640 | 1280 | 1437 | 1613 | 0.80 |
| PUO218 | 1280 | 640 | 320 | 640 | 320 | 160 | 640 | 640 | 640 | 1280 | 320 | 1280 | 508 | 640 | 0.44 |
| JAH | 640 | 640 | 320 | 1280 | 160 | 320 | 640 | 640 | 320 | 640 | 320 | 320 | 359 | 570 | 0.17 |
| PR159 | 640 | 1280 | 320 | 1280 | 320 | 320 | 640 | 1280 | 160 | 640 | 160 | 320 | 320 | 718 | 0.050 |
| YF/DEN3 | 1280 | 2560 | 640 | 640 | 320 | 640 | 1280 | 1280 | 640 | 1280 | 320 | 640 | 640 | 1016 | 0.26 |
| PaH88/81 | 640 | 1280 | 320 | 320 | 320 | 320 | 640 | 640 | 320 | 320 | 320 | 1280 | 403 | 570 | 0.22 |
| 1301 | 320 | 640 | 80 | 40 | 160 | 80 | 160 | 160 | 40 | 40 | 160 | 320 | 127 | 127 | 0.57 |
| 1325 | 160 | 320 | 40 | 80 | 80 | 160 | 80 | 160 | 40 | 40 | 160 | 160 | 80 | 127 | 0.21 |
| YF/DEN4 | 2560 | 5120 | 2560 | 5120 | 2560 | 5120 | 5120 | 5120 | 5120 | 5120 | 2560 | 5120 | 3225 | 5120 | 0.01 |
| 1228 | 640 | 640 | 320 | 1280 | 320 | 320 | 640 | 1280 | 320 | 640 | 320 | 640 | 403 | 718 | 0.057 |
| BC26-97 | 1280 | 1280 | 5120 | 1280 | 1280 | 1280 | 1280 | 2560 | 1280 | 1280 | 1280 | 1280 | 1613 | 1437 | 0.54 |
| P75-215 | 320 | 640 | 160 | 640 | 40 | 160 | 80 | 160 | 80 | 80 | 40 | 40 | 90 | 180 | 0.20 |

^{a}Obtained 61 days after the first dose and prior to the second dose (Day 63).
^{b}Obtained 31 days after the second dose.
^{c}The 50% neutralization titers against individual dengue viruses (Table 1) were compared by t tests using sera obtained pre and post booster immunization. Bold numbers in the P value column represent statistically significant increases in levels of antibodies between pre and post immunization sera measured against individual DEN strains.

TABLE 18
Viremia in monkeys immunized with 5 log₁₀ PFU (S.C.)
of different clones of ChimeriVax-DEN1 viruses

| | Virus | Viremia (log₁₀ PFU/ml) by post-immunization day: | | | | | | | | | |
|---|---|---|---|---|---|---|---|---|---|---|---|
| Monkey | (AA change) | 2* | 3 | 4 | 5 | 6 | 7 | 8 | 9 | 10 | 11 |
| R18265M | YF/DEN-1, 99, | —** | — | — | — | — | — | — | — | — | — |
| R175110F | P4, uncloned | — | — | — | 1.7 | — | — | — | — | — | — |
| F17572M | (M39, E204) | 1.3 | 1.0 | — | 1.0 | — | — | — | — | — | — |
| F171114F | | — | — | — | — | — | — | — | — | — | — |
| R182104M | YF/DEN-1, 01, | 1.0 | 1.9 | 1.7 | 1.7 | 1.8 | 1.7 | 1.0 | 1.0 | 1.7 | — |
| R175108F | P7, clone J, | — | 1.7 | 2.8 | 2.2 | 1.0 | 2.0 | 1.7 | 2.0 | 2.2 | 1.7 |
| R182111M | PMS (none) | 2.3 | 3.0 | 3.3 | 2.8 | 1.7 | 1.7 | — | — | — | — |
| R175104F | | — | 2.4 | 1.3 | 2.0 | 2.3 | 1.7 | 1.7 | 2.2 | 3.0 | 3.1 |
| R182103M | YF/DEN-1, 01, | — | — | — | — | — | — | — | — | — | — |
| R17098F | P6, clone E | — | 1.7 | — | — | — | — | — | — | — | — |
| R18261M | (E204) | 1.7 | 2.5 | 1.3 | 2.0 | | | | | | — |
| R175118F | | — | — | 1.0 | — | — | — | — | — | — | — |

*Monkeys were immunized on Day 1 via subcutaneous injection using 5.0 log₁₀ PFU.
**<1.0 log₁₀ PFU/ml

TABLE 19
Summary of viremia and neutralizing antibody titers
(50%) in monkeys immunized with 5 log₁₀ PFU (S.C.)
of different clones of ChimeriVax-DEN1 viruses

| | | No. viremic/ | Mean | | Neut. |
|---|---|---|---|---|---|
| | | no. tested | Peak | | Ab |
| Monkey | Mutation | (%) | titer | Duration | Titer* |
| R18265M | YF-DEN1, 99, | 2/4 (50) | 1.5 | 1.5 | 640 |
| R175110F | P4, uncloned | | | | 640 |
| R17572M | (M39, E204) | | | | 320 |
| R171114F | | | | | 640 |
| R182103M | YF-DEN1, 01, | 3/4 (75) | 1.7 | 2 | 5120 |
| R17098F | P6, clone E | | | | 2560 |
| R18261M | (E204) | | | | 2560 |
| R175118F | | | | | 5120 |
| R182104M | YF-DEN1, 01, | 4/4 (100) | 2.5 | 8.5 | 5120 |
| R175108F | P7, clone J, | | | | 10240 |
| R182111M | PMS (None) | | | | 10240 |
| R175104F | | | | | 10240 |

*Measured 30 days post immunization.

TABLE 20
Treatment Groups

| | Number | | Nominal Dose | Dose |
|---|---|---|---|---|
| Group | of | Vaccine | Level | Volume |
| No. | ♂/♀ | Preparation | (PFU*) | (ml) |
| 1 | 2/1 | DEN1 (P10) | ~10⁵ | 0.5 |
| 2 | 1/2 | DEN2 (P10) | ~10⁵ | 0.5 |
| 3 | 2/1 | DEN3 (P10) | ~10⁵ | 0.5 |
| 4 | 2/1 | DEN4 (P10) | ~10⁵ | 0.5 |
| 5 | 1/2 | DEN2 cGMP | ~10⁵ | 0.5 |
| | | vaccine, | | |
| | | Reference | | |
| | | Article | | |
| 6 | 1/2 | DEN1, DEN2, | ~10⁵ each | 2.0 |
| | | DEN3, DEN4** | (~4 × 10⁵ total) | (1 ml to |
| | | | | each arm) |
| 7 | 2/1 | YF-Vax ®, | ~1.1 × 10⁵*** | 0.5 |
| | | Control Article | | |

*PFU = plaque-forming units.
**Provided by Acambis, Inc. as a mixture of four viral suspensions (~4 × 10⁵ PFU/2 ml).
***The dose level described in Protocol Amendment 1 incorrectly described the nominal dose as ~10⁵ PFU and should have read ~1.1 × 10⁵ PFU, according to the dose concentration provided by manufacturer and the desired dose volume prescribed in the protocol.

TABLE 21
Summary, Viremia in cynomolgus monkeys immunized
s.c. with one dose of ChimeriVax-DEN (P10)
monovalent or tetravalent formulations

| | | | Mean | |
|---|---|---|---|---|
| | Dose (log₁₀ | No. | Peak viremia | duration |
| Virus | PFU/ml) | viremic | (log₁₀ PFU/ml) | (Days) |
| YF/DEN1 | 5 | 1/3 | 2.7 | 6 |
| YF/DEN2 | 5 | 1/3 | 2.0 | 3 |
| YF/DEN3 | 5 | 3/3 | 1.8 | 3 |
| YF/DEN4 | 5 | 3/3 | 2.1 | 4.3 |
| YF/DEN2 CO (GMP) | 5 | 3/3 | 1.8 | 2.3 |
| YF-VAX | 5 | 3/3 | 1.7 | 2.3 |
| YF/DEN1-4 | (5, 5, 5, 5) | 3/3 | 1.9 | 3.7 |

TABLE 22
PRNT₅₀ of sera of monkeys immunized
with monovalent (P10) vaccines

| | | PRNT₅₀ vs. | PRNT₅₀ vs. |
|---|---|---|---|
| Monkey # | Vaccine | homologous virus | WT parent |
| F20515M | YF/DEN1 | >10240 | 2560 |
| F205116F | | >10240 | 320 |
| F18612M | | >10240 | 1280 |
| F205105F | YF/DEN2 | 1280 | 640 |
| F20514M | | >10240 | 160 |
| F19997F | | >10240 | 320 |
| F20502M | YF/DEN3 | 5120 | 160 |
| F20571F | | 5120 | 640 |
| F20500M | | 1280 | 640 |
| F20513M | YF/DEN4 | 2560 | 40 |
| F205109F | | 2560 | 160 |
| F18131M | | 160 | 20 |
| F20589F | YF/DEN2 | 1280 | 80 |
| F20509M | GMP | 320 | 160 |
| F20511F | vaccine | 640 | 160 |
| F18910M | YF-VAX | 320 | ND |
| F20575F | | 640 | |
| F19673M | | 320 | |

TABLE 23
Neutralizing antibody titers in cynomolgus monkeys immunized
SC with one dose of tetravalent DEN (P10) vaccine preparation

| | Viral Strains used in Assay | | | | | | | |
|---|---|---|---|---|---|---|---|---|
| Monkey# | DEN1 | Puo-359* | DEN2 | PUO-218 | DEN3 | PaH881 | DEN4 | 1228 |
| F20587F | 320 | 5120 | 640 | 160 | 1280 | 160 | 1280 | 160 |
| F20504M | 320 | 1280 | 320 | 320 | 640 | 40 | 2560 | 320 |
| F20594F | 160 | 2560 | 320 | 320 | 640 | 40 | 2560 | 640 |

*Values obtained in repeat test

TABLE 24
Neurovirulence, in 4 day old suckling mice inoculated by the i.c. route,
of ChimeriVax-DEN1- and DEN3 P10 viruses isolated from monkeys

| | | | | AA change | | | |
|---|---|---|---|---|---|---|---|
| | | | | from | Dose | | |
| | | Day | Size | inoculated | Dilution/BT | Mortality | AST |
| Monkey | Vaccine | isolated | (mm) | vaccine | (log₁₀ PFU/ml | (dead/total) | (Days) |
| F20515F | YF/DEN1 | 10 | 1 | E204 R > N | Neat/2.2 | 5/11 | 12.2 |
| | | | | | 1:10/1.2 | 3/11 | 11.3 |
| F20515F | YF/DEN1 | 10 | 5 | E351 V > L | Neat/2.3 | 10/11 | 10.9 |
| F20502F | YF/DEN3 | 7 | 2 | E202 K > R | Neat/3.2 | 0/10 | NA |
| | | | | | 1:10/2.2 | 0/10 | NA |
| F20502F | YF/DEN3 | 7 | 1 | — | Neat/0* | 0/10 | NA |
| F20502F | | | | | 1:10/−1 | 0/10 | NA |
| NA | YF-VAX ® | NA | NA | NA | 1:20/1.7 | 11/11 | 8.45 |

*Back titration of inocula used in suckling mice revealed an unexpected low titer of 50 PFU/ml. The calculated titer (0.02 ml dose) inoculated into suckling mice was therefore 1 PFU = 0 log_{10..} We did not attempt to re-isolate this plaque from monkey serum and repeat the suckling mouse test because the genome sequence of this plaque (small) was not altered in vivo.

TABLE 25
Total viremia in cynomolgus monkeys immunized s.c. with
one dose of ChimeriVax-DEN1-4 GMP Vaccine viruses

| | | Viremia (log₁₀ PFU/ml) by Day post immunization: | | | | | | | | | |
|---|---|---|---|---|---|---|---|---|---|---|---|
| Group | Monkey | 2^{a} | 3 | 4 | 5 | 6 | 7 | 8 | 9 | 10 | 11 |
| 5, 5, | F20967F | —b | 2.3 | — | — | — | — | — | — | — | — |
| 5, 5 | F21343M | 1.7 | 1.7 | — | 1.7 | 2.0 | 2.2 | 2.0 | — | — | — |
| | F21786F | 1.7 | 2.8 | 1.7 | 1.7 | 2.2 | 1.7 | 1.7 | — | — | — |
| | F213114F | 2.0 | 2.9 | 2.0 | — | — | — | — | — | — | — |
| | F21339M | — | 2.8 | 2.0 | — | 1.7 | — | — | — | — | — |
| | F21386F | — | 2.7 | 2.2 | 1.7 | — | 1.7 | — | — | — | — |
| 3, 5, | F21501M | — | — | — | — | — | — | — | — | — | 1.7 |
| 5, 3 | F212117F | — | — | — | — | — | — | — | — | — | 2.7 |
| | F21355M | 2.0 | 2.6 | 1.7 | 2.2 | 2.2 | 1.7 | — | — | 2.6 | — |
| | F20977F | — | — | — | — | — | — | — | — | 1.7 | — |
| | F21534M | — | — | — | — | — | — | — | — | — | 2.5 |
| | F21565F | 1.7 | 3.2 | 2.2 | — | — | — | — | — | — | 1.7 |
| 5, 5, | F21342M | 2.3 | 3.1 | 2.3 | — | — | — | — | 1.7 | 2.0 | — |
| 5, 3 | F212105F | 1.7 | 2.2 | — | — | 1.7 | — | — | — | 2.0 | 1.7 |
| | F21544M | 1.7 | — | — | — | — | — | — | — | — | — |
| | F21784F | — | 2.2 | — | — | — | 1.7 | — | — | — | 2.0 |
| | F21149M | — | — | — | — | 2.0 | — | — | 1.7 | — | — |
| | F21384F | — | 1.7 | — | — | 1.7 | — | — | — | — | — |
| 3, 3, | F209108F | — | — | — | 1.7 | 2.5 | 2.4 | 2.8 | 2.0 | — | — |
| 3, 3 | F21311M | — | — | 1.7 | — | 2.5 | 2.8 | 2.4 | 2.5 | 2.7 | 2.0 |
| | F18172F | — | — | 2.2 | — | 2.7 | 2.0 | 1.7 | — | — | 1.7 |
| | F20788F | — | 1.7 | — | 2.0 | 2.5 | 2.5 | 2.4 | — | — | — |
| | F21522M | — | — | — | — | 1.7 | — | — | 1.7 | 2.0 | — |
| | F21570F | — | — | 2.4 | 2.4 | 2.3 | 2.0 | 2.3 | 2.2 | 2.4 | 2.2 |

^{a}Monkeys were immunized on Day 1.
b: <1.7 log₁₀ PFU/ml

TABLE 26
Summary, Total Viremia in cynomolgus monkeys
immunized s.c. with one dose of ChimeriVax-DEN1-4
GMP Vaccine tetravalent formulations

| | Dose | No. Viremic/ | Viremia | |
|---|---|---|---|---|
| Group | (log10 PFU) | Total | Mean Peak | Mean Duration |
| 1 | 5, 5, 5, 5 | 6/6 | 2.6 | 4.0 |
| 2 | 3, 5, 5, 3 | 6/6 | 2.4 | 2.5 |
| 3 | 5, 5, 5, 3 | 6/6 | 2.1 | 3.0 |
| 4 | 3, 3, 3, 3 | 6/6 | 2.5 | 5.5 |

TABLE 27
Identification of ChimeriVax ™-DEN serotypes in viremic monkeys

| | | ChimeriVax ™-DEN serotypes detected by day post immunization: | | | | | | | | | |
|---|---|---|---|---|---|---|---|---|---|---|---|
| Group | Monkey | 2^{a} | 3 | 4 | 5 | 6 | 7 | 8 | 9 | 10 | 11 |
| 5, 5, 5, 5 | F20967F | —^{b} | 3 | — | 4 | — | — | — | — | — | — |
| | F21343M | 4 | 1-4^{c} | 3 | 1, 3 | 4 | 3, 4 | — | — | — | — |
| | F21786F | 1, 4 | 1-4 | 1-4 | 3, 4 | 4 | 4 | — | — | — | — |
| | F213114F | 4 | 1-4 | 3, 4 | 3 | — | — | — | — | — | — |
| | F21339M | — | 1, 3, 4 | 4 | — | — | — | — | — | — | — |
| | F21386F | — | 1-4 | 1, 3, 4 | 4 | 4 | 4 | | 4 | | |
| 3, 5, 5, 3 | F21501M | — | 3 | 3 | 3 | 3 | — | — | — | — | — |
| | F212117F | — | — | — | — | — | — | — | — | — | 4 |
| | F21355M | 2, 3, 4 | 2, 3, 4 | 2, 3 | 4 | 4 | 4 | 4 | 4 | 3, 4 | 3 |
| | F20977F | — | 3 | 2, 3 | — | 3 | 3 | 3 | 3 | — | 2 |
| | F21534M | — | — | — | — | — | — | — | — | — | 2 |
| | F21565F | 2, 3 | 2, 3 | 3 | — | — | — | — | — | — | — |
| 5, 5, 5, 3 | F21342M | 1, 2, 3 | 1, 2, 3 | 1, 3 | — | — | — | — | 4 | 4 | — |
| | F212105F | 1, 2, 3 | 1, 2, 3 | 3 | — | — | 4 | — | 4 | 4 | — |
| | F21544M | — | — | — | — | — | — | 3 | — | 3 | 4 |
| | F21784F | — | 1, 2, 3 | 2, 3, 4 | — | — | — | — | — | | — |
| | F21149M | — | — | — | — | — | — | — | — | 2 | — |
| | F21384F | 1, 4 | 1, 2, 3 | 2, 3, 4 | 3 | 1, 2 | 2, 4 | 4 | 2, 4 | 4 | 4 |
| 3, 3, 3, 3 | F209108F | — | — | — | — | 1-4 | 1, 3, 4 | 1, 3, 4 | 3, 4 | — | — |
| | F21311M | — | 2, 3 | 2, 3, 4 | 2, 3, 4 | 1, 2, 4 | 1, 2, 4 | 1, 2, 4 | 1-4 | 3, 4 | 4 |
| | F18172F | — | 4 | 1-4 | 1-4 | 1-4 | 1-4 | 4 | — | — | — |
| | F20788F | — | 2 | — | 1, 2, 4 | 2 | 2 | — | 2 | 4 | — |
| | F21522M | 2 | 2, 3, 4 | 2 | 2, 3, 4 | 2, 3, 4 | 4 | — | 4 | 4 | — |
| | F21570F | — | — | 2,3 | 3 | 4 | 3, 4 | 4 | 4 | 3, 4 | 3, 4 |

^{a}Monkeys were immunized on Day 1.
^{b}<1.3 log₁₀ PFU/ml.
^{c}1 = ChimeriVax ™-DEN1, 2 = ChimeriVax ™-DEN2, 3 = ChimeriVax ™-DEN3, 4 = ChimeriVax ™-DEN4.

TABLE 28
Summary, magnitude and duration of serotype specific
viremia in monkeys immunized with ChimeriVax ™-DEN1-4
tetravalent formulations

Mean

| | | Peak Viremia | |
|---|---|---|---|
| | ChimeriVax ™- | (Log₁₀ | Duration |
| Formulation | DEN | IFFU/ml) | (Days) |
| 5, 5, 5, 5 | 1 | 1.5 | 1.8 |
| | 2 | 1.6 | 1.2 |
| | 3 | 2.3 | 2.3 |
| | 4 | 1.9 | 3.7 |
| 3, 5, 5, 3 | 1 | 0 | 0 |
| | 2 | 2.3 | 2.0 |
| | 3 | 2.6 | 4.5 |
| | 4 | 2.7 | 4.5 |
| 5, 5, 5, 3 | 1 | 1.8 | 2.2 |
| | 2 | 1.7 | 2.4 |
| | 3 | 2.2 | 2.6 |
| | 4 | 2.1 | 2.8 |
| 3, 3, 3, 3 | 1 | 1.9 | 3.0 |
| | 2 | 1.9 | 3.8 |
| | 3 | 2.0 | 4.2 |
| | 4 | 2.5 | 5.3 |

TABLE 29
PRNT₅₀ of sera (against homologous viruses) in monkeys immunized with tetravalent DEN GMP vaccines

| | | | PRNT 50% against ChimeriVax ™-DEN1-4 viruses by post immunization day: | | | | | | | | | | |
|---|---|---|---|---|---|---|---|---|---|---|---|---|---|
| | | DEN1 | | | DEN2 | | | DEN3 | | | DEN4 | | |
| | | Day | Day | Day | Day | Day | Day | Day | Day | Day | Day | Day | Day |
| Formulation | Monkey | 1 | 31 | 121 | 1 | 31 | 121 | 1 | 31 | 121 | 1 | 31 | 121 |
| 5, 5, 5, 5 | F20967F | <10 | 1280 | 160 | 10 | 5120 | 320 | <10 | 2560 | 640 | <10 | 1280 | 320 |
| | F21343M | 10 | 160 | 640* | <10 | 160 | 320 | <10 | 160 | 1280 | <10 | 160 | 320 |
| | F21786F | <10 | 1280 | 2560 | <10 | 1280 | 2560 | <10 | 320 | 2560 | <10 | 1280 | 2560 |
| | F213114F | <10 | 640 | 320 | <10 | 160 | 40 | <10 | 160 | 640 | <10 | 320 | 160 |
| | F21339M | <10 | 160 | 160 | <10 | 640 | 1280 | <10 | 640 | 640 | <10 | 640 | 1280 |
| | F21386F | <10 | 320 | 160 | <10 | 160 | 160 | <10 | 640 | 1280 | <10 | 320 | 320 |
| 3, 3, 3, 3 | F209108F | <10 | 320 | 40 | <10 | 640 | 80 | <10 | 320 | 160 | <10 | 160 | 40 |
| | F21311M | <10 | 160 | 20 | <10 | 320 | 640 | <10 | 320 | 20 | <10 | 160 | 10 |
| | F18172F | <10 | 640 | 40 | <10 | 160 | 640 | <10 | 320 | 320 | <10 | 1280 | 320 |
| | F20788F | <10 | 1280 | 80 | <10 | 2560 | 640 | <10 | 320 | 640 | <10 | 1280 | 40 |
| | F21522M | <10 | 80 | 160 | <10 | 640 | 40 | <10 | 80 | 320 | <10 | 640 | <10* |
| | F21570F | <10 | 80 | 160 | <10 | 40 | 1280 | <10 | 640 | 1280 | <10 | 320 | 320 |
| 3, 5, 5, 3 | F21501M | <10 | 160 | 160 | 10 | 1280 | 1280 | <10 | 640 | 640 | <10 | 40 | 160 |
| | F212117F | <10 | 20 | 80 | <10 | 40 | 640 | <10 | 20 | 320 | <10 | <10 | 640 |
| | F21355M | <10 | 40 | 20 | <10 | 320 | 20 | <10 | 640 | 40 | <10 | 160 | 10 |
| | F20977F | <10 | 20 | 320 | <10 | 320 | 640 | <10 | 640 | 640 | <10 | <10 | <10 |
| | F21534M | <10 | 1280 | 40 | <10 | 20 | 1280 | <10 | 40 | 10 | <10 | <10 | <10 |
| | F21565F | 10 | 160 | 320 | <10 | 5120 | 640 | <10 | 1280 | 320 | <10 | 160 | 640 |
| 5, 5, 5, 3 | F21342M | <10 | 1280 | 320 | <10 | 5120 | 1280 | <10 | 10240 | 160 | <10 | 320 | 80 |
| | F212105F | <10 | 320 | 160 | <10 | 160 | 1280 | <10 | 320 | 320 | <10 | 20 | 1280* |
| | F21544M | 10 | 40 | 640 | <10 | 40 | 160 | <10 | 80 | 320 | <10 | <10 | 320 |
| | F21784F | <10 | 160 | 80 | <10 | 640 | 320 | <10 | 5120 | 640 | <10 | 160 | 1280 |
| | F21149M | <10 | 40 | 40 | <10 | 640 | 320 | <10 | <10 | 160 | <10 | <10 | 2560* |
| | F21384F | <10 | 1280 | 80 | <10 | 640 | 640 | <10 | 320 | 640 | <10 | 320 | 1280 |

*values obtained by repeat test. Values shown in bold numbers are ≧10

TABLE 30
Summary, immunogenicity in cynomolgus monkeys immunized SC with one dose of
ChimeriVax-DEN1-4 GMP Vaccine tetravalent formulation (GMTs DAY 31 and 120)

| | No. seroconvert. | | GMT 50% PRNT ± SD against ChimeriVax ™-DEN1-4 viruses by post immunization day: | | | | | | | | | | | |
|---|---|---|---|---|---|---|---|---|---|---|---|---|---|---|
| | To all Den1-4 | | DEN1 | | | DEN2 | | | DEN3 | | | DEN4 | | |
| | Day | Day | Day | Day | Day | Day | Day | Day | Day | Day | Day | Day | Day | Day |
| Dose | 31 | 121 | 1 | 31 | 121 | 1 | 31 | 121 | 1 | 31 | 121 | 1 | 31 | 121 |
| 5, 5, 5, 5 | 6/6 | 6/6 | 1.5 ± | 452 ± | 359 ± | 1.5 ± | 508 ± | 359 ± | 1 ± | 452 ± | 1016 ± | 1 ± | 508 ± | 508 ± |
| | | | 3.7 | 526 | 946 | 3.7 | 1945 | 977 | 0 | 914 | 748 | 0 | 500 | 941 |
| 3, 3, 3, 3 | 6/6 | 5/6^{b} | 1± | 254 ± | 63 ± | 1 ± | 359 ± | 320 ± | 1 ± | 285 ± | 254 ± | 1 ± | 452 ± | 34 ± |
| | | | 0 | 468 | 62 | 0 | 931 | 456 | 0 | 178 | 453 | 0 | 526 | 154 |
| 3, 5, 5, 3 | 3/6^{c} | 4/6^{d} | 1.5 ± | 90 ± | 101 ± | 1.5 ± | 285 ± | 452 ± | 1 ± | 254 ± | 160 ± | 1 ± | 10 ± | 29 ± |
| | | | 3.7 | 494 | 135 | 3.7 | 1982 | 476 | 0 | 469 | 275 | 0 | 78 | 314 |
| 5, 5, 5, 3 | 4/6^{e} | 6/6 | 1.5 ± | 226 ± | 142 ± | 1 ± | 452 ± | 508 ± | 1 ± | 275 ± | 320 ± | 1 ± | 26 ± | 718 ± |
| | | | 3.7 | 598 | 229 | 0 | 1936 | 500 | 0 | 4200 | 219 | 0 | 154 | 880 |

^{a}For calculation of GMTs titers <10 were assigned 1.
^{b}Monkey F21522M became seronegative to DEN4.
^{c}Three monkeys (F212117F, F20977F, and F21534M) were seronegative to DEN4.
^{d}Monkey F212117 became seropositive to DEN4.
^{e}Two monkeys (F21544M and F21149M) were seronegative to DEN4. Values shown in bold are GMTs for day 31 and 121.

TABLE 31
Protection of monkeys immunized with ChimeriVax-DEN1-4 tetravalent
formulations and challenged* with heterologous WT dengue 1

| | | Viremia by day post challenge: | | | | | | | | | | PRNT50 | |
|---|---|---|---|---|---|---|---|---|---|---|---|---|---|
| Monkey | Vaccine | 2 | 3 | 4 | 5 | 6 | 7 | 8 | 9 | 10 | 11 | Prechal. | Postchal. |
| F20967F | 5, 5, 5, 5 | 0** | 0 | 0 | 0 | 0 | 0 | 0 | 0 | 0 | 0 | 160 | 5,120 |
| F21501M | 3, 5, 5, 3 | 0 | 0 | 0 | 0 | 0 | 0 | 0 | 0 | 0 | 0 | 40 | 20,480 |
| F21784F | 5, 5, 5, 3 | 0 | 0 | 0 | 0 | 0 | 0 | 0 | 0 | 0 | 0 | 40 | 20,480 |
| F18172F | 3, 3, 3, 3 | 0 | 0 | 0 | 0 | 0 | 0 | 0 | 0 | 0 | 0 | 80 | 20,480 |
| F21149M | 5, 5, 5, 3 | 0 | 0 | 0 | 0 | 2.0 | 3.0 | 3.3 | 1.7 | 0 | 0 | 20 | 20,480 |
| F21570F | 3, 3, 3, 3 | 0 | 0 | 0 | 0 | 0 | 0 | 0 | 0 | 0 | 0 | 160 | 10,240 |
| F20928M | None | 0 | 1.0 | 1.7 | 2.7 | 3.0 | 2.8 | 0 | 0 | 0 | 0 | <10 | 2,560 |
| F22673F | None | 0 | 0 | 1.0 | 1.3 | 2.9 | 3.3 | 3.2 | 1.7 | 0 | 0 | <10 | 1,280 |
| F21753M | None | 0 | 0 | 2.0 | 2.7 | 2.9 | 3.4 | 3.4 | 1.7 | 1.0 | 0 | <10 | 2,560 |
| F21143F | None | 0 | 1.0 | 1.7 | 1.7 | 2.5 | 3.3 | 2.6 | 0 | 0 | 0 | <10 | >10,240 |

*All monkeys were challenged with 5 logs of Dengue 1 (West Pacific 74, non attenuated) with the exception of monkeys F20928M and F22673F (Group 5 SBi report) which received 4 logs of challenge virus (pilot study).
**No virus could be detected when serum was tested in plaque assay at undiluted, 1:2 or 1:10 dilutions, limit of detection = 1 log₁₀ pfu/ml.

TABLE 32
Protection of monkeys immunized with different ChimeriVax-DEN1-4 tetravalent
formulations and challenged* with heterologous WT dengue 2 (incomplete)

| | | PRNT50 | | Viremia by day post challenge: | | | | | | | | | |
|---|---|---|---|---|---|---|---|---|---|---|---|---|---|
| Monkey | Vaccine | Prechall | Postchall | 2 | 3 | 4 | 5 | 6 | 7 | 8 | 9 | 10 | 11 |
| F212117F | 5, 5, 5, 5 | | | 0** | 0 | 0 | 0 | 0 | 0 | 0 | 0 | 0 | 0 |
| F21342M | 3, 5, 5, 3 | | | 0 | 0 | 0 | 0 | 0 | 0 | 0 | 0 | 0 | 0 |
| F21786F | 5, 5, 5, 3 | | | 0 | 0 | 0 | 0 | 0 | 0 | 0 | 0 | 0 | 0 |
| F21384F | 3, 3, 3, 3 | | | 0 | 0 | 0 | 0 | 0 | 0 | 0 | 0 | 0 | 0 |
| F21544M | 5, 5, 5, 3 | | | 0 | 0 | 0 | 0 | 0 | 0 | 0 | 0 | 0 | 0 |
| F21386F | 3, 3, 3, 3 | | | 0 | 0 | 0 | 0 | 0 | 0 | 0 | 0 | 0 | 0 |
| F22605M | None | <10 | | 0 | 0 | 0 | 0 | 0 | 0 | 2.0 | 2.5 | 2.7 | 2.2 |
| F226100F | None | <10 | | 0 | 0 | 0 | 0 | 0 | 1.0 | 2.5 | 2.3 | 0 | 0 |
| F21563F | None | <10 | | 0 | 0 | 0 | 0 | 1.7 | 2.4 | 2.0 | 2.3 | 3.0 | 1.7 |
| F21550M | None | <10 | | 0 | 0 | 0 | 0 | 0 | 1.3 | 1.3 | 1.0 | 1.8 | 1.6 |

*All monkeys were challenged with 5 logs of Dengue 2 (S16803), PDK10 with the exception of monkeys F22605M and F226100F (Group 6 SBi report) which received 4 logs of challenge virus (pilot study).
**No virus could be detected when serum was tested in plaque assay at undiluted, 1:2 or 1:10 dilutions. Limit of detection = 1 log₁₀ pfu/ml.

TABLE 33
Protection of monkeys immunized with different ChimeriVax-DEN1-4
formulations and challenged* with heterologous WT dengue 3

| | | Viremia by day post challenge: | | | | | | | | | | PRNT50 | |
|---|---|---|---|---|---|---|---|---|---|---|---|---|---|
| Monkey | Vaccine | 2 | 3 | 4 | 5 | 6 | 7 | 8 | 9 | 10 | 11 | Prechal. | Postchal. |
| F21343M | 5, 5, 5, 5 | 0** | 0 | 0 | 0 | 0 | 0 | 0 | 0 | 0 | 0 | 320 | 20,480 |
| F21339 M | 5, 5, 5, 5 | 0 | 0 | 0 | 0 | 0 | 0 | 0 | 0 | 0 | 0 | 640 | 5,120 |
| F212105F | 5, 5, 5, 3 | 0 | 0 | 0 | 0 | 0 | 0 | 0 | 0 | 0 | 0 | 1,280 | 10,240 |
| F20788F | 3, 3, 3, 3 | 0 | 0 | 0 | 0 | 0 | 0 | 0 | 0 | 0 | 0 | 640 | 40,960 |
| F21522M | 3, 3, 3, 3 | 0 | 0 | 0 | 0 | 0 | 0 | 0 | 0 | 0 | 0 | 640 | 2,560 |
| F21565F | 3, 5, 5, 3 | 0 | 0 | 0 | 0 | 0 | 0 | 0 | 0 | 0 | 0 | 1280 | 10,240 |
| F22127M | None | 0 | 1.6 | 1.0 | 1.5 | 1.6 | 1.7 | 0 | 0 | 0 | 0 | <10 | >20,480 |
| F20992F | None | 0 | 1.7 | 1.3 | 0 | 1.3 | 2.5 | 0 | 0 | 0 | 0 | <10 | 2,560 |
| F22607M | None | 0 | 0 | 0 | 1.7 | 0 | 1.0 | 2.0 | 1.7 | 0 | 0 | <10 | 10,240 |
| F22606M | None | 0 | 1.7 | 0 | 0 | 0 | 0 | 0 | 2.5 | 2.0 | 2.0 | <10 | 10,240 |

*All monkeys were challenged with 5 logs of Dengue 3 (D3 (CH53489, PS) with the exception of monkeys F22127M and F20992F (Group 7 SBi report) which received 4 logs of challenge virus (pilot study).
**No virus was detected when serum was tested in plaque assay at undiluted, 1:2 or 1:10 dilutions, limit of detection = 1 log₁₀ pfu/ml.

TABLE 34
Protection of monkeys immunized with different ChimeriVax-DEN-1-4 tetravalent
formulations and challenged* with heterologous WT dengue 4 (incomplete)

| | | PRNT50 | | Viremia by day post challenge: | | | | | | | | | |
|---|---|---|---|---|---|---|---|---|---|---|---|---|---|
| Monkey | Vaccine | Prechall | Postchall | 2 | 3 | 4 | 5 | 6 | 7 | 8 | 9 | 10 | 11 |
| F21311M | 3, 3, 3, 3 | | | 0 | 0 | 0 | 0 | 0 | 0 | 0 | 0 | 0 | 0 |
| F209108F | 3, 3, 3, 3 | | | 0 | 0 | 0 | 0 | 0 | 0 | 0 | 0 | 0 | 0 |
| F21355M | 3, 5, 5, 3 | | | 0 | 0 | 0 | 0 | 0 | 0 | 0 | 0 | 0 | 0 |
| F213114F | 5, 5, 5, 5 | | | 0 | 0 | 0 | 0 | 0 | 0 | 0 | 0 | 0 | 0 |
| F21534M | 3, 5, 5, 3 | | | 0 | 0 | 0 | 0 | 0 | 0 | 0 | 0 | 0 | 0 |
| F20977F | 3, 5, 5, 3 | | | 0 | 0 | 0 | 0 | 0 | 1.7 | 0 | 1.7 | 0 | 0 |
| F22612M | None | <10 | | 0 | 0 | 0 | 0 | 1.0 | 0 | 1.5 | 2.3 | 2.5 | 3.1 |
| F207107F | None | <10 | | 0 | 0 | 0 | 1.0 | 2.0 | 2.9 | 2.7 | 2.0 | 0 | 0 |
| F21752M | None | <10 | | 0 | 0 | 0 | 2.2 | 3.2 | 3.6 | 2.9 | 0 | 0 | 0 |
| F21102F | None | <10 | | 0 | 0 | 0 | 1.7 | 2.4 | 2.6 | 3.3 | 2.9 | 2.4 | 1.8 |

*All monkeys were challenged with 5 logs of Dengue 4 (Carib, 341750, non attenuated) with the exception of monkeys F22612M and F207107F (Groups 8 SBi report) which received 4 logs of challenge virus (pilot study).
**No virus was detected when serum was tested in plaque assay at undiluted, 1:2 or 1:10 dilutions, limit of detection = 1 log₁₀ pfu/ml.

TABLE 35
Protection of ChimeriVax-DEN tetravalent immunized
monkeys upon challenge with WRAIR WT dengue viruses

| | | | | Mean Viremia | | |
|---|---|---|---|---|---|---|
| | | | No. | Peak | | % Protected |
| | | Challenge | Viremic/ | (log₁₀ | Duration | (Sterile |
| Group | Vaccinated | Virus | Total | Pfu/ml) | (days) | Immunity*) |
| 5 | Yes | Dengue 1 | 1/6 | 3.3 | 4.4 | 83 |
| 6 | No | ″ | 4/4 | 3.2 | 6.0 | 0 |
| 7 | Yes | Dengue 2 | 0/6 | — | — | 100 |
| 8 | No | ″ | 4/4 | 2.5 | 4.5 | 0 |
| 9 | Yes | Dengue 3 | 0/6 | — | — | 100 |
| 10 | No | ″ | 4/4 | 2.2 | 4.2 | 0 |
| 11 | Yes | Dengue 4 | 1/6 | 1.7 | 2 | 83 |
| 12 | No | ″ | 4/4 | 3.2 | 5.2 | 0 |

*No virus could be found in a plaque assay on Vero cells using undiluted, 1:2 or 1:10 dilutions of sera obtained from Day 2 to Day 11.

TABLE 36
Treatment Groups

| | | | | Target Dose |
|---|---|---|---|---|
| | | | | Suspension |
| Group | Number of | | Target Dose Level | Concentration |
| No. | Male/Female | Treatment | (PFU or TCID₅₀)¹ | (PFU or TCID₅₀/mL) |
| 1 | 6/5 | YF-Vax ® | ~5.5 × 10⁴ PFU | ~2.2 × 10⁵ PFU/mL |
| | | (Commercial | | |
| | | Yellow Fever | | |
| | | Vaccine) | | |
| 2 | 5/6 | Tetravalent | ~1.0 × 10⁵ (TCID₅₀) | ~4.0 × 10⁵ TCID₅₀/mL |
| | | ChimeriVax ™ - | of ChimeriVax ™-DEN1, | of ChimeriVax ™-DEN1, |
| | | DEN Suspension | ChimeriVax ™-DEN2, | ChimeriVax ™-DEN2, |
| | | | ChimeriVax ™-DEN3, | ChimeriVax ™-DEN3, |
| | | | and ChimeriVax ™-DEN4 | and ChimeriVax ™-DEN4 |

PFU = plaque-forming units and TCID₅₀ = tissue culture infectious dose

TABLE 37
Mean Lesion Scores for Cynomolgus Monkeys following
Intracerebral Inoculation with YF-Vax ®

| | Target | Discriminator | Combined |
|---|---|---|---|
| Monkey No. | areas | areas | scores |
| F20538M | 0.69 | 0.69 | 0.69 |
| F209115F | 0.92 | 1.04 | 0.98 |
| F20930M | 0 | 0 | 0 |
| F20994F | 0.58 | 0.13 | 0.36 |
| F21353M | 0.3 | 0.19 | 0.11 |
| F21706M | 1.25 | 0.25 | 0.75 |
| F21708M | 0.5 | 0.5 | 0.5 |
| F217104F | 0.26 | 0.38 | 0.32 |
| F21721M | 0.11 | 0.12 | 0.13 |
| F21774F | 0.03 | 0.06 | 0.05 |
| F21768F | 1.11 | 0.5 | 0.81 |
| Mean | 0.52 | 0.35 | 0.43 |
| Standard | 0.43 | 0.31 | 0.34 |
| Deviation | | | |

TABLE 38
Mean Lesion Scores for Cynomolgus Monkeys following
Intracerebral Inoculation with Tetravalent
ChimeriVax-Den Vaccine Preparation

| | Target | Discriminator | Combined |
|---|---|---|---|
| Monkey No. | areas | areas | scores |
| F19933M | 0 | 0 | 0 |
| F207113F | 0.17 | 0 | 0.09 |
| F209105F | 0.03 | 0.19 | 0.11 |
| F209116F | 0 | 0 | 0 |
| F20986F | 0 | 0 | 0 |
| F21524M | 0 | 0.06 | 0.03 |
| F21535M | 0.03 | 0.06 | 0.05 |
| F21711M | 0.17 | 0 | 0.09 |
| F217116F | 0 | 0 | 0 |
| F21754M | 0 | 0 | 0 |
| F21790F | 0.17 | 0.06 | 0.12 |
| Mean | 0.05 | 0.03 | 0.04 |
| Standard Deviation | 0.08 | 0.06 | 0.05 |
| p value* | 0.0023 | 0.0012 | 0.0023 |

*Scores of YF-Vax group vs Chimerivax ™-DEN1-4 group (Kruskall-Wallis test)

TABLE 39
Summary of total serum viremia in monkeys inoculated IC with
YF-Vax ® or ChimeriVax ™-DEN1-4

| | | Mean peak | Mean |
|---|---|---|---|
| | | viremia | duration |
| Vaccine | Dose | (log₁₀ PFU/mL) | (days) |
| YF-Vax ® | ~5.5 × 10⁴ PFU | 2.1 | 3 |
| ChimeriVax- | ~1.0 × 10⁵ TCID₅₀ | 2.7 | 6 |
| DEN1-4 | each | | |

TABLE 40
Group assignment

| | Number of | | Dose |
|---|---|---|---|
| Group | Males/ | | Level |
| No. | Females | Treatment | (PFU)^{a} |
| 1 | 3/3 | YF-Vax ® | ~5.5 × 10⁴ |
| | | (Commercial Yellow Fever Vaccine) | |
| 2 | 3/3 | ChimeriVax ™-Dengue 1 | ~1.0 × 10⁵ |
| | | Pre-Master Seed (Clone J-2-P7) | |
| | | Vaccine | |
| 3 | 3/3 | ChimeriVax ™-Dengue 1 Bulk Vaccine | ~1.0 × 10⁵ |

^{a}PFU = plaque-forming units

What is claimed is:

1. A method of inducing an immune response to the four serotypes of dengue virus in a patient, the method comprising administering to the patient a vaccine comprising:
a chimeric flavivirus comprising the capsid and non-structural proteins of Yellow Fever virus and the pre-membrane and envelope proteins of Dengue-1 virus;
a chimeric flavivirus comprising the capsid and non-structural proteins of Yellow Fever virus and the pre-membrane and envelope proteins of Dengue-2 virus;
a chimeric flavivirus comprising the capsid and non-structural proteins of Yellow Fever virus and the pre-membrane and envelope proteins of Dengue-3 virus; and
a chimeric flavivirus comprising the capsid and non-structural proteins of Yellow Fever virus and the pre-membrane and envelope proteins of Dengue-4 virus.

2. The method of claim 1, wherein all four chimeras are administered at equivalent concentrations.

3. The method of claim 2, wherein each chimera is administered at a concentration of 5 log₁₀ PFU.

4. The method of claim 2, wherein each chimera is administered at a concentration of 4 log₁₀ PFU.

5. The method of claim 1, wherein the Dengue-1 and Dengue-2 chimeras are administered at an amount that is greater than that of the Dengue-3 and Dengue-4 chimeras.

6. The method of claim 5, wherein said Dengue-1 and Dengue-2 chimeras are administered at 5 log₁₀ PFU and said Dengue-3 and Dengue-4 chimeras are administered at 4 log₁₀ PFU.

7. The method of claim 5, wherein said Dengue-1 and Dengue-2 chimeras are administered at 5 log₁₀ PFU and said Dengue-3 and Dengue-4 chimeras are administered at 3 log₁₀PFU.

8. The method of claim 5, wherein said Dengue-1 and Dengue-2 chimeras are administered at 4 log₁₀ PFU and said Dengue-3 and Dengue-4 chimeras are administered at 3 log₁₀ PFU.

9. The method of claim 1, wherein said patient does not have, but is at risk of developing, Dengue infection.

10. The method of claim 1, wherein said patient has Dengue infection.

11. A vaccine composition comprising:
a chimeric flavivirus comprising the capsid and non-structural proteins of Yellow Fever virus and the pre-membrane and envelope proteins of Dengue-1 virus;
a chimeric flavivirus comprising the capsid and non-structural proteins of Yellow Fever virus and the pre-membrane and envelope proteins of Dengue-2 virus;
a chimeric flavivirus comprising the capsid and non-structural proteins of Yellow Fever virus and the pre-membrane and envelope proteins of Dengue-3 virus; and
a chimeric flavivirus comprising the capsid and non-structural proteins of Yellow Fever virus and the pre-membrane and envelope proteins of Dengue-4 virus.

12. The composition of claim 11, wherein all four chimeras are present in said composition in equivalent concentrations.

13. The composition of claim 12, wherein each chimera is present at a concentration of 5 log₁₀PFU.

14. The composition of claim 12, wherein each chimera is present at a concentration of 4 log₁₀ PFU.

15. The composition of claim 11, wherein the Dengue-1 and Dengue-2 chimeras are present in an amount that is greater than that of the Dengue-3 and Dengue-4 chimeras.

16. The composition of claim 15, wherein said Dengue-1 and Dengue-2 chimeras are present at 5 log₁₀ PFU and said Dengue-3 and Dengue-4 chimeras are present at 4 log₁₀ PFU.

17. The composition of claim 15, wherein said Dengue-1 and Dengue-2 chimeras are present at 5 log₁₀ PFU and said Dengue-3 and Dengue-4 chimeras are present at 3 log₁₀ PFU.

18. The composition of claim 15, wherein said Dengue-1 and Dengue-2 chimeras are present at 4 log₁₀ PFU and said Dengue-3 and Dengue-4 chimeras are present at 3 log₁₀ PFU.
